(19) United States
(12) Patent Application Publication
(10) Pub. No.: US 2026/0151228 A1
(43) Pub. Date: Jun. 4, 2026

(54) DEVICES AND SYSTEMS FOR DOCKING A HEART VALVE

(71) Applicant: Edwards Lifesciences Corporation, Irvine, CA (US)

(72) Inventors: Tyler Dale O'Dell, Irvine, CA (US); Rachel Christine Leacock, Torrance, CA (US); Bich Hoang Pham, Irvine, CA (US); Vivian Tran, Santa Ana, CA (US); Taylor Michael Winters, Stevenson Ranch, CA (US); Alison Louise Rodriguez, Irvine, CA (US); Abdorruhman M. Yousef, Santa Ana, CA (US); Anthony Michael Romero, Corona, CA (US)

(21) Appl. No.: 19/456,988

(22) Filed: Jan. 22, 2026

Related U.S. Application Data
(63) Continuation of application No. PCT/US2024/038964, filed on Jul. 22, 2024.
(60) Provisional application No. 63/515,127, filed on Jul. 23, 2023.

(51) Int. Cl. A61F 2/24

(52) U.S. Cl. CPC A61F 2/2418; A61F 2220/0008; A61F 2220/0075; A61F 2250/0071; A61F 2250/0098

(57) ABSTRACT

A docking station for a medical device includes an expandable frame having a central waist portion defining a seat and a cover secured to the expandable frame and covering at least the waist portion of the expandable frame. The cover includes proximal and distal cover portions overlapping to define a medial pocket region, a suture stitched through the medial pocket region and including circumferential portions and axially extending peripheral portions defining a plurality of pockets in the medial pocket region, and a plurality of radiopaque markers, each disposed in a corresponding one of the plurality of pockets.

### RELATED APPLICATION

The present application is a continuation of PCT Patent Application No. PCT/US2024/038964, filed on Jul. 22, 2024, which application claims priority to and all benefit of U.S. Provisional Patent Application Ser. No. 63/515,127, filed on Jul. 23, 2023, for DEVICES AND SYSTEMS FOR DOCKING A HEART VALVE, the disclosure of each of these applications being incorporated herein by reference in its entirety.

### FIELD

The present disclosure relates to heart valves and, in particular, docking stations/stents, delivery systems, and methods for use in implanting a heart valve, e.g., a transcatheter heart valve (“THV”).

### BACKGROUND

Prosthetic heart valves can be used to treat cardiac valvular disorders. The native heart valves (the aortic, pulmonary, tricuspid and mitral valves) serve critical functions in assuring the forward flow of an adequate supply of blood through the cardiovascular system. These heart valves can be rendered less effective by congenital, inflammatory, or infectious conditions. Such conditions can eventually lead to serious cardiovascular compromise or death. For many years the definitive treatment for such disorders was the surgical repair or replacement of the valve during open heart surgery.

A transcatheter technique can also be used for introducing and implanting a prosthetic heart valve using a flexible catheter in a manner that is less invasive than open heart surgery. In this technique, a prosthetic valve can be mounted in a crimped state on the end portion of a flexible catheter and advanced through a blood vessel of the subject until the valve reaches the implantation site. The valve at the catheter tip can then be expanded to its functional size at the site of the defective native valve, such as by inflating a balloon on which the valve is mounted. Alternatively, the valve can have a resilient, self-expanding stent or frame that expands the valve to its functional size when it is advanced from a delivery sheath at the distal end of the catheter.

Transcatheter heart valves (THVs) can be appropriately sized to be placed inside most native aortic valves. However, with larger native valves, blood vessels, and grafts, aortic transcatheter valves might be too small to secure into the larger implantation or deployment site. In this case, the transcatheter valve may not be large enough to sufficiently expand inside the native valve or other implantation or deployment site to be secured in place.

Replacing the pulmonary valve, which is sometimes referred to as the pulmonic valve, presents significant challenges. The geometry of the pulmonary artery can vary greatly from patient to patient. Typically, the pulmonary artery outflow tract after corrective surgery is too wide for effective placement of a prosthetic heart valve.

### SUMMARY

This summary is meant to provide examples and is not intended to be limiting of the scope of the invention in any way. For example, any feature included in an example of this summary is not required by the claims, unless the claims explicitly recite the feature. The description discloses exemplary embodiments of expandable docking stations for an expandable valve. The docking stations can be constructed in a variety of ways.

In some implementations, a docking station for a medical device includes an expandable frame extending axially from a proximal end to a distal end and having a central waist portion defining a seat, and a cover secured to the expandable frame and covering at least the waist portion of the expandable frame.

In some implementations, the cover includes a proximal cover portion having an inner axial end disposed at the central waist portion of the expandable frame and an outer axial end extending toward the proximal end of the expandable frame, and a distal cover portion having an inner axial end disposed at the central waist portion of the expandable frame and an outer axial end extending toward the distal end of the expandable frame, with the inner axial end of the distal cover portion overlapping with the inner axial end of the proximal cover portion to define a medial pocket region.

In some implementations, the cover further includes a suture stitched through the medial pocket region, the suture having circumferential portions and axially extending peripheral portions defining a plurality of pockets in the medial pocket region.

In some implementations, the cover includes a plurality of radiopaque markers, each disposed in a corresponding one of the plurality of pockets.

In some implementations, one of the proximal cover portion and the distal cover portion includes a plurality of axially inward extending flaps secured to the other of the proximal cover portion and the distal cover portion to define the plurality of pockets.

In some implementations, the proximal and distal cover portions each include a plurality of axially inward extending flaps that align to define the plurality of pockets.

In some implementations, each pocket includes a crossing suture portion extending through an aperture in the corresponding radiopaque marker to secure the radiopaque marker within the pocket.

In some implementations, the crossing suture portion includes interlocking stitches.

In some implementations, the crossing suture portion is continuous with the peripheral suture portion.

In some implementations, the suture is a first suture and the crossing suture portion is defined by a second suture separate from the first suture.

In some implementations, the crossing suture portion further extends through a second aperture in the corresponding radiopaque marker to secure the radiopaque marker within the pocket.

In some implementations, the suture includes a first suture pattern and a second suture pattern.

In some implementations, wherein the first and second suture patterns include non-interlocking stitches in the peripheral portions of the suture.

In some implementations, the first and second suture patterns include interlocking stitches in the peripheral portions of the suture.

In some implementations, the first and second suture patterns include interlocking stitches in the circumferential portions of the suture.

In some implementations, the first and second suture patterns include non-interlocking stitches in the circumferential portions of the suture.

In some implementations, the first and second suture patterns include interlocking crossing stitches extending through an aperture in the radiopaque marker.

In some implementations, the circumferential portions of the suture are axially aligned with the pockets.

In some implementations, the circumferential portions of the suture are axially offset from the pockets.

In some implementations, the suture restricts the central waist portion to a first diameter.

In some implementations, the suture includes a plurality of frangible portions disposed between a plurality of reinforced portions, such that when an expansion tool is inserted into the waist portion of the expandable frame and expanded to a size larger than the first diameter, the frangible portions of the suture break without breakage of the reinforced portions to expand the waist portion to a second diameter larger than the first diameter.

In some implementations, the plurality of frangible portions is disposed in the circumferential portions of the suture.

In some implementations, the plurality of reinforced portions includes the peripheral portions of the suture.

In some implementations, the cover further includes reinforcing junctions between the plurality of frangible portions and the plurality of reinforced portions.

In some implementations, the reinforcing junctions include suture knots.

In some implementations, the inner axial ends of the proximal and distal cover portions include axially inward extending flaps that overlap to define a plurality of pockets between the proximal cover portion and the distal cover portion, with a plurality of radiopaque markers, each disposed in a corresponding one of the plurality of pockets.

In some implementations, the axial inner ends of the proximal and distal covers overlap, the cover further including a suture stitched through the overlapping axial inner ends.

In some implementations, the suture includes circumferential portions and axially extending peripheral portions extending through the plurality of pockets.

In some implementations, a method of forming a cover for a docking station frame includes overlapping an inner axial end of a proximal cover portion with an inner axial end of a distal cover portion to define a medial pocket region.

In some implementations, the method includes positioning a plurality of radiopaque markers in the medial pocket region between the proximal cover portion and the distal cover portion.

In some implementations, the method includes sewing a suture through the medial pocket region, the suture including circumferential portions and axially extending peripheral portions defining a plurality of pockets between the inner axial ends of the proximal and distal cover portions, with each of the plurality of radiopaque markers being disposed in a corresponding one of the plurality of pockets.

In some implementations, a docking station for a medical device includes an expandable frame having a waist portion defining a seat for retaining an expandable medical device and a cover secured to the expandable frame and covering at least the waist portion of the expandable frame, the cover including a circumferential band extending around the waist portion of the expandable frame to restrict expansion of the waist portion to a first diameter.

In some implementations, the circumferential band includes a plurality of frangible portions disposed between a plurality of reinforced portions, such that when an expansion tool is inserted into the waist portion of the expandable frame and expanded to a size larger than the first diameter, the frangible portions of the circumferential band break without breakage of the reinforced portions to expand the waist portion to a second diameter larger than the first diameter.

In some implementations, the circumferential band comprises a suture.

In some implementations, a method is provided for installing an expandable transcatheter valve in a docking station having an expandable frame including a waist portion defining a seat for retaining an expandable medical device, and a cover secured to the expandable frame and covering at least the waist portion of the expandable frame, the cover including a circumferential band extending around the waist portion of the expandable frame to restrict expansion of the waist portion to a first diameter.

In some implementations, the method includes inserting an expansion tool into the waist portion of the expandable frame and expanding the expansion tool to break frangible portions of the circumferential band, thereby expanding the waist portion from the first diameter to a second diameter larger than the first diameter.

In some implementations, the method includes expanding the expandable transcatheter valve within the expanded waist portion of the docking station frame to secure the expandable transcatheter valve within the docking station.

In some implementations, the docking station further includes an original transcatheter valve installed in the waist portion of the docking station frame.

In some implementations, inserting the expansion tool into the waist portion of the expandable frame comprises inserting the expansion tool into a central passage of the original transcatheter valve.

In some implementations, expanding the expansion tool comprises expanding the central passage of the original transcatheter valve.

In some implementations, expanding the expandable transcatheter valve within the expanded waist portion of the docking station frame comprises expanding the expandable transcatheter valve into seating engagement with the central passage of the original transcatheter valve.

In some implementations, the circumferential band comprises a suture.

Various embodiments and methods described herein can be utilized within a subject in various procedures, including (but not limited to) medical and training procedures. Subjects include (but are not limited to) medical patients, veterinary patients, animal models, cadavers, and simulators of the cardiac and vasculature system (e.g., anthropomorphic phantoms and explant tissue).

Various features as described elsewhere in this disclosure can be included in the examples summarized here and various methods and steps for using the examples and features can be used, including as described elsewhere herein.

Further understanding of the nature and advantages of the disclosed inventions can be obtained from the following description and claims, particularly when considered in conjunction with the accompanying drawings in which like parts bear like reference numerals.

### BRIEF DESCRIPTION OF THE DRAWINGS

To further clarify various aspects of embodiments of the present disclosure, a more particular description of the certain embodiments will be made by reference to various aspects of the appended drawings. It is appreciated that these drawings depict only typical embodiments of the present disclosure and are therefore not to be considered limiting of the scope of the disclosure. Moreover, while the figures may be drawn to scale for some embodiments, the figures are not necessarily drawn to scale for all embodiments. Embodiments of the present disclosure will be described and explained with additional specificity and detail through the use of the accompanying drawings.

FIG. 1A is a cutaway view of the human heart in a diastolic phase;

FIG. 1B is a cutaway view of the human heart in a systolic phase;

FIGS. 2A and 2B are sectional views of pulmonary arteries illustrating that pulmonary arteries can have a variety of different shapes and sizes;

FIG. 3A is a cutaway view of the human heart in a systolic phase with a docking station deployed in a pulmonary artery;

FIG. 3B is a cutaway view of the human heart in a systolic phase with a docking station and transcatheter heart valve deployed in a pulmonary artery;

FIG. 4A is an enlarged schematic illustration of the docking station and transcatheter heart valve of FIG. 3B when the heart is in the systolic phase;

FIG. 4B is a view taken in the direction indicated by lines 4B-4B in FIG. 4A;

FIG. 4C is a graph showing a relationship between a docking station diameter and a radial outward force applied by the docking station;

FIG. 5 is a cutaway view of the human heart in a diastolic phase with a docking station and transcatheter heart valve deployed in a pulmonary artery;

FIG. 6A is an enlarged schematic illustration of the docking station and transcatheter heart valve of FIG. 5 when the heart is in the diastolic phase;

FIG. 6B is a view taken in the direction indicated by lines 6B-6B in FIG. 6A;

FIG. 7A illustrates an exemplary embodiment of a docking station with a transcatheter heart valve disposed inside the docking station;

FIG. 7B illustrates an exemplary embodiment of a docking station with a transcatheter heart valve disposed inside the docking station;

FIG. 7C illustrates an exemplary embodiment of a docking station with a transcatheter heart valve disposed inside the docking station;

FIG. 7D illustrates an exemplary embodiment of a docking station with a transcatheter heart valve disposed inside the docking station;

FIG. 7E illustrates an exemplary embodiment of a docking station with a transcatheter heart valve disposed inside the docking station;

FIG. 7F illustrates an exemplary embodiment of a docking station with a transcatheter heart valve disposed inside the docking station;

FIG. 7G illustrates an exemplary embodiment of a docking station with a transcatheter heart valve disposed inside the docking station;

FIG. 8A is a side view of an exemplary embodiment of a frame of a docking station;

FIG. 8B illustrates a side profile of the frame illustrated by FIG. 8A;

FIG. 9 illustrates the docking station frame of FIG. 8A in a compressed state;

FIG. 10A is a perspective view of the docking station frame of FIG. 8A;

FIG. 10B is a perspective view of the docking station frame of FIG. 8A;

FIG. 11 is a perspective view of an exemplary embodiment of a docking station having a plurality of covered cells and a plurality of open cells;

FIG. 12 is a perspective view of the docking station illustrated by FIG. 11 with a portion cut away to illustrate a transcatheter heart valve expanded into place in the docking station;

FIG. 13 illustrates a side profile of the docking station illustrated by FIG. 11 when implanted in a vessel of the circulatory system;

FIG. 14 illustrates a perspective view of the docking station illustrated by FIG. 11 when installed in a vessel of the circulatory system;

FIG. 15 illustrates a perspective view of the docking station and valve illustrated by FIG. 12 when implanted in a vessel of the circulatory system;

FIGS. 16A and 16B illustrate side profiles of the docking station illustrated by FIG. 18 when implanted in different size vessels of the circulatory system;

FIGS. 17 and 18 illustrate side profiles of the docking station illustrated by FIG. 11 when implanted in different sized vessels of the circulatory system with a schematically illustrated transcatheter heart valve having the same size installed or deployed in each docking station;

FIG. 19A is a sectional view illustrating a side profile of an exemplary embodiment of a docking station placed in a pulmonary artery;

FIG. 19B is a sectional view illustrating a side profile of an exemplary embodiment of a docking station placed in a pulmonary artery and a schematically illustrated valve placed in the docking station;

FIG. 19C is a sectional view illustrating an exemplary embodiment of a docking station placed in a pulmonary artery and a valve placed in the docking station;

FIG. 20A is a cutaway view of the human heart in a systolic phase with a docking station deployed in a pulmonary artery;

FIG. 20B is a cutaway view of the human heart in a systolic phase with a docking station and transcatheter heart valve deployed in a pulmonary artery;

FIG. 21A is an enlarged schematic illustration of the docking station and transcatheter heart valve of FIG. 20B when the heart is in the systolic phase;

FIG. 21B is a view taken in the direction indicated by lines 21B-21B in FIG. 21A;

FIG. 22 is a cutaway view of the human heart, docking station, and transcatheter heart valve deployed in the pulmonary artery illustrated by FIG. 20B when the heart is in the diastolic phase;

FIG. 23A is an enlarged schematic illustration of the docking station and transcatheter heart valve of FIG. 22 when the heart is in the diastolic phase;

FIG. 23B is a view taken in the direction indicated by lines 23B-23B in FIG. 23A;

FIG. 24A is a cutaway view of the human heart in a systolic phase with a docking station being deployed in a pulmonary artery;

FIG. 24B is a cutaway view of the human heart in a systolic phase with a docking station deployed in a pulmonary artery;

FIG. 24C is a cutaway view of the human heart in a systolic phase with a docking station and transcatheter heart valve deployed in a pulmonary artery;

FIG. 25A is an enlarged schematic illustration of the docking station and transcatheter heart valve of FIG. 24C when the heart is in the systolic phase;

FIG. 25B is a view taken in the direction indicated by lines 25B-25B in FIG. 25A;

FIG. 26 is a cutaway view of the human heart, docking station, and transcatheter heart valve deployed in the pulmonary artery illustrated by FIG. 24C when the heart is in the diastolic phase;

FIG. 27A is an enlarged schematic illustration of the docking station and transcatheter heart valve of FIG. 25A when the heart is in the diastolic phase;

FIG. 27B is a view taken in the direction indicated by lines 27B-27B in FIG. 27A;

FIGS. 28-31, and 32A-32C illustrate examples of valve types that can be deployed in a docking station, e.g., one of the docking stations described or depicted herein;

FIG. 33 illustrate deployment of a docking station from a catheter;

FIG. 34 is a side view of one embodiment of a frame of a docking station;

FIG. 35 is a side view of another embodiment of a frame of a docking station;

FIG. 36A is a side view of one embodiment of a frame of a docking station;

FIG. 36B is a bottom view of the frame of FIG. 36A;

FIG. 36C is a top view of the frame of FIG. 36A;

FIG. 37A is a side view of another embodiment of a frame of a docking station;

FIG. 37B is a bottom view of the frame of FIG. 37A;

FIG. 37C is a top view of the frame of FIG. 37A;

FIG. 38A is a side view of one embodiment of a docking station having outflow cells;

FIG. 38B is a top view of the docking station of FIG. 38A;

FIG. 39 is a side view of another embodiment of a docking station having outflow cells;

FIG. 40A is a side view of a frame of a docking station of one embodiment;

FIG. 40B is a side view of a frame of a docking station of another embodiment;

FIG. 40C is a side view of a frame of a docking station of another embodiment;

FIG. 41A is a side view of a docking station with a frame and an impermeable material according to one embodiment,

FIG. 41B is a side view of a docking station with a frame and an impermeable material according to another embodiment,

FIG. 41C is a side view of a docking station with a frame and an impermeable material according to another embodiment;

FIG. 41D is a side view of a docking station with a frame and an impermeable material according to another embodiment;

FIG. 42A is a top component of an impermeable material having a proximal portion and a distal portion;

FIG. 42B is a side perspective view of the assembled proximal portion of the impermeable material of FIG. 42A;

FIG. 42C is a top perspective view of the assembled proximal portion of the impermeable material of FIG. 42A;

FIG. 42D is a side perspective view of the assembled distal portion of the impermeable material of FIG. 42A;

FIGS. 42E-42I are side perspective views of the assembly of the impermeable material of FIG. 42A;

FIG. 42J is a top schematic view of the proximal portion and the distal portion of FIG. 42A outlined on a cloth according to one embodiment;

FIG. 42K is a top schematic view of the proximal portion and the distal portion of FIG. 42A outlined on a cloth according to another embodiment;

FIG. 43 is a side view of an impermeable material of one embodiment disposed within a frame of one embodiment;

FIGS. 44A-44I, illustrate one method of affixing the impermeable material and frame of FIG. 43 to one another;

FIG. 45A is a side perspective view of a radiopaque marker according to one embodiment;

FIG. 45B is a side perspective view of a radiopaque marker according to another embodiment;

FIG. 45C is a side perspective view of a radiopaque marker according to another embodiment;

FIG. 45D is a side perspective view of a radiopaque marker according to another embodiment;

FIG. 46A is a perspective view of an impermeable material with radiopaque markers according to one embodiment;

FIG. 46B is a side view of an impermeable material with radiopaque markers disposed in pockets;

FIG. 46C is a schematic illustration of a pocket covering disposed over a pocket of an impermeable material according to one embodiment;

FIG. 46D is a schematic illustration of a pocket covering disposed over a pocket of an impermeable material according to another embodiment;

FIGS. 46E-46H illustrate a method of affixing a pocket and a radiopaque marker to an impermeable member;

FIG. 46I illustrates an additional step in the method of FIGS. 46E-46H according to one embodiment;

FIG. 46J illustrates an additional step in the method of FIGS. 46E-46H according to another embodiment;

FIG. 46K illustrates an additional step in the method of FIGS. 46E-46H according to another embodiment;

FIG. 46L illustrates an additional step in the method of FIGS. 46E-46H according to another embodiment;

FIG. 46M illustrates an additional step in the method of FIGS. 46E-46H according to another embodiment;

FIG. 46N illustrates an additional step in the method of FIGS. 46E-46H according to another embodiment;

FIG. 47 is a side perspective view of a cover with radiopaque markers retained in pocket portions of the cover, according to another embodiment;

FIG. 48 is a side perspective view of a cover with radiopaque markers retained in pocket portions of the cover, according to another embodiment;

FIG. 48A is a plan view of a cover portion providing a flap shaped to form a pocket portion of a cover, according to another embodiment;

FIG. 49 is a side perspective view of a cover with radiopaque markers retained in pocket portions of the cover, according to another embodiment;

FIG. 50 is a schematic view of axial inner portions of proximal and distal portions of a cover, shown prior to attachment, according to another embodiment;

FIG. 51 is a schematic view of the axial inner portions of the cover of FIG. 50, shown sutured together to form a pocket retaining a radiopaque marker, according to another embodiment;

FIG. 51A is a schematic view of axial inner portions of the proximal and distal portions of a cover, shown sutured together to form a pocket retaining a radiopaque marker, according to another embodiment;

FIGS. 52A-52E illustrate a method of affixing a radiopaque marker between flap portions of proximal and distal portions of a cover, according to another embodiment;

FIGS. 53A-53F illustrate another method of affixing a radiopaque marker between flap portions of proximal and distal portions of a cover, according to another embodiment;

FIGS. 54A-54F illustrate another method of affixing a radiopaque marker between flap portions of proximal and distal portions of a cover, according to another embodiment;

FIGS. 55A-55E illustrate another method of affixing a radiopaque marker between flap portions of proximal and distal portions of a cover, according to another embodiment;

FIGS. 56A-56E illustrate another method of affixing a radiopaque marker between flap portions of proximal and distal portions of a cover, according to another embodiment;

FIGS. 57A-57E illustrate another method of affixing a radiopaque marker between flap portions of proximal and distal portions of a cover, according to another embodiment;

FIGS. 58A-58E illustrate another method of affixing a radiopaque marker between flap portions of proximal and distal portions of a cover, according to another embodiment;

FIG. 59 is a side perspective view of a cover with radiopaque markers retained in pocket portions of the cover and a waist restricting suture arrangement, according to another embodiment;

FIG. 60 is a side perspective view of a cover having a waist restricting suture arrangement, according to another embodiment;

FIG. 61 is a cutaway view of the human heart with a docking station having a cover with a waist restricting suture arrangement and a transcatheter heart valve deployed in a pulmonary artery;

FIG. 62 is an enlarged view of the docking station, transcatheter heart valve, and pulmonary artery of FIG. 61, shown with a noncompliant expanded balloon enlarging the waist portions of the docking station and transcatheter heart valve; and

FIG. 63 is an enlarged view of the docking station, transcatheter heart valve, and pulmonary artery of FIG. 61, shown with a catheter installing a replacement transcatheter heart valve in the enlarged waist portions of the docking station and original transcatheter heart valve.

### DETAILED DESCRIPTION

The following description refers to the accompanying drawings, which illustrate specific embodiments of the invention. Other embodiments having different structures and operation do not depart from the scope of the present invention. Exemplary embodiments of the present disclosure are directed to devices and methods for providing a docking station or landing zone for a transcatheter heart valve (“THV”), e.g., THV 29. In some exemplary embodiments, docking stations for THVs are illustrated as being used within the pulmonary artery, although the docking stations (e.g., docking station 10) can be used in other areas of the anatomy, heart, or vasculature, such as the superior vena cava or the inferior vena cava. The docking stations described herein can be configured to compensate for the deployed THV being smaller than the space (e.g., anatomy/vasculature/etc.) in which it is to be placed. Other exemplary embodiments for use with the features and implementations described herein are described and shown in PCT patent application no. PCT/US2021/019770, filed on Feb. 26, 2021, which application claims the benefit of U.S. Provisional Application No. 62/991,687, filed on Mar. 19, 2020, and U.S. Provisional Application No. 63/137,619, filed on Jan. 14, 2021, the content of each of these applications being incorporated herein by reference in their entireties.

Prosthetics, including docking stations, may be utilized in a variety of subjects and procedures. Subjects include (but are not limited to) medical patients, veterinary patients, animal models, cadavers, and simulators of the cardiac and vasculature system (e.g., anthropomorphic phantoms and explant tissue). Procedures include (but are not limited to) medical and training procedures.

It should be noted that various embodiments of docking stations and systems for delivery and implant are disclosed herein, and any combination of these options can be made unless specifically excluded. For example, any of the docking stations devices disclosed, can be used with any type of valve, and/or any delivery system, even if a specific combination is not explicitly described. Likewise, the different constructions of docking stations and valves can be mixed and matched, such as by combining any docking station type/feature, valve type/feature, tissue cover, etc., even if not explicitly disclosed. In short, individual components of the disclosed systems can be combined unless mutually exclusive or otherwise physically impossible.

For the sake of uniformity, in these figures and others in the application the docking stations are depicted such that the pulmonary bifurcation end is up, while the ventricular end is down. These directions may also be referred to as “distal” as a synonym for up or the pulmonary bifurcation end, and “proximal” as a synonym for down or the ventricular end, which are terms relative to the physician's perspective.

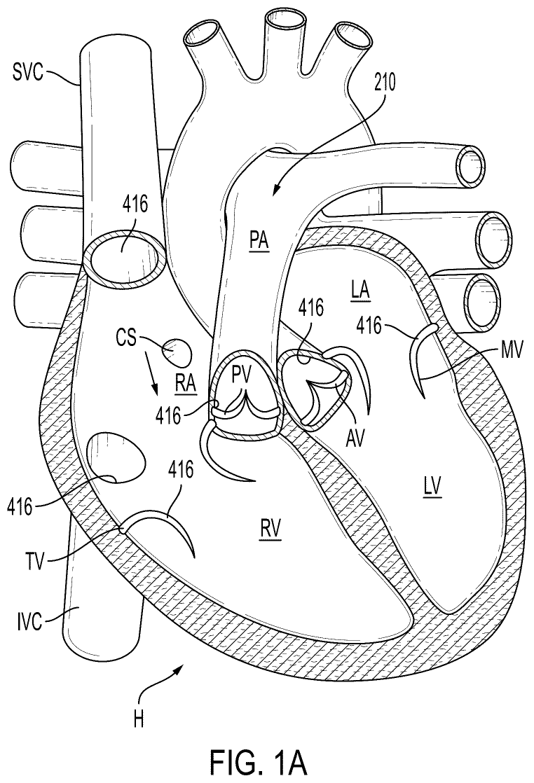

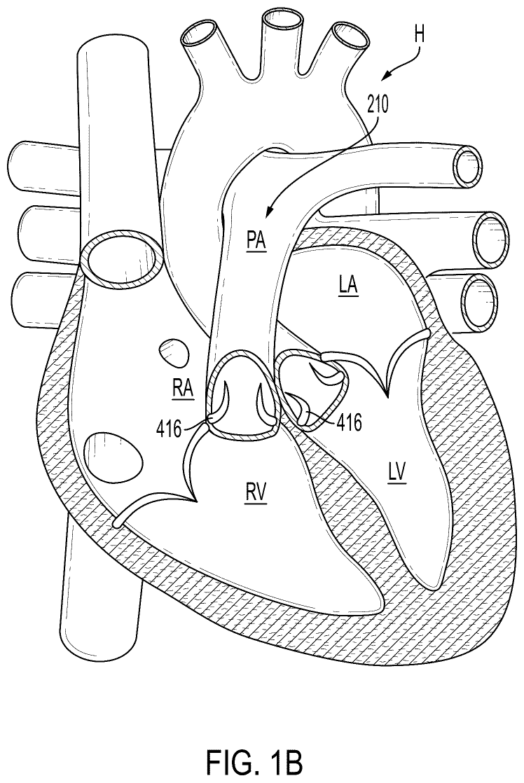

FIGS. 1A and 1B are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta (not identified) and the pulmonary valve PV separates the right ventricle from the pulmonary artery PA. Each of these valves has flexible leaflets extending inward across the respective orifices that come together or “coapt” in the flowstream to form the one-way, fluid-occluding surfaces. The docking stations and valves of the present application are described primarily with respect to the pulmonary valve. Therefore, anatomical structures of the right atrium RA and right ventricle RV will be explained in greater detail. It should be understood that the devices described herein can also be used in other areas, e.g., in the inferior vena cava and/or the superior vena cava as treatment for a regurgitant or otherwise defective tri-cuspid valve, in the aorta (e.g., an enlarged aorta) as treatment for a defective aortic valve, in other areas of the heart or vasculature, in grafts, etc.

The right atrium RA receives deoxygenated blood from the venous system through the superior vena cava SVC and the inferior vena cava IVC, the former entering the right atrium from above, and the latter from below. The coronary sinus CS is a collection of veins joined together to form a large vessel that collects deoxygenated blood from the heart muscle (myocardium), and delivers it to the right atrium RA. During the diastolic phase, or diastole, seen in FIG. 1A, the venous blood that collects in the right atrium RA enters the tricuspid valve TV by expansion of the right ventricle RV. In the systolic phase, or systole, seen in FIG. 1B, the right ventricle RV contracts to force the venous blood through the pulmonary valve PV and pulmonary artery into the lungs. In one exemplary embodiment, the devices described by the present application are used to replace or supplement the function of a defective pulmonary valve. During systole, the leaflets of the tricuspid valve TV close to prevent the venous blood from regurgitating back into the right atrium RA.

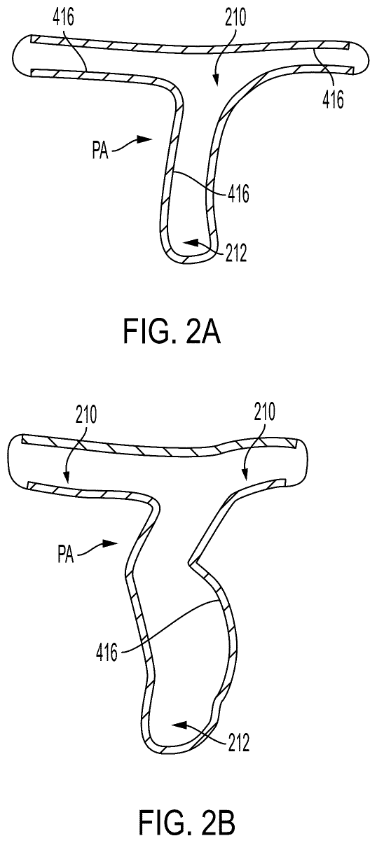

Referring to FIGS. 2A and 2B, the shown, non-exhaustive examples illustrate that the pulmonary artery can have a wide variety of different shapes and sizes. For example, as shown in the sectional views of FIGS. 2A and 2B, the length L, diameter, D, and curvature or contour may vary greatly between pulmonary arteries of different patients. Further, the diameter D may vary significantly along the length L of an individual pulmonary artery. These differences can be even more significant in pulmonary arteries that suffer from certain conditions and/or have been compromised by previous surgery. For example, the treatment of Tetralogy of Fallot (TOF) or Transposition of the Great Arteries (TGA) often results in larger and more irregularly shaped pulmonary arteries.

Tetralogy of Fallot (TOF) is a cardiac anomaly that refers to a combination of four related heart defects that commonly occur together. The four defects are ventricular septal defect (VSD), overriding aorta (the aortic valve is enlarged and appears to arise from both the left and right ventricles instead of the left ventricle as in normal hearts), pulmonary stenosis (narrowing of the pulmonary valve and outflow tract or area below the valve that creates an obstruction of blood flow from the right ventricle to the pulmonary artery), and right ventricular hypertrophy (thickening of the muscular walls of the right ventricle, which occurs because the right ventricle is pumping at high pressure).

Transposition of the Great Arteries (TGA) refers to an anomaly where the aorta and the pulmonary artery are “transposed” from their normal position so that the aorta arises from the right ventricle and the pulmonary artery from the left ventricle.

Surgical treatment for some conditions involves a longitudinal incision along the pulmonary artery, up to and along one of the pulmonary branches. This incision can eliminate or significantly impair the function of the pulmonary valve. A trans-annular patch is used to cover the incision after the surgery. The trans-annular patch reduces stenotic or constrained conditions of the pulmonary artery PA, associated with other surgeries. However, the impairment or elimination of the pulmonary valve PV can create significant regurgitation and, prior to the present invention, often required later open-heart surgery to replace the pulmonary valve. The trans-annular patch technique can result in pulmonary arteries having a wide degree of variation in size and shape.

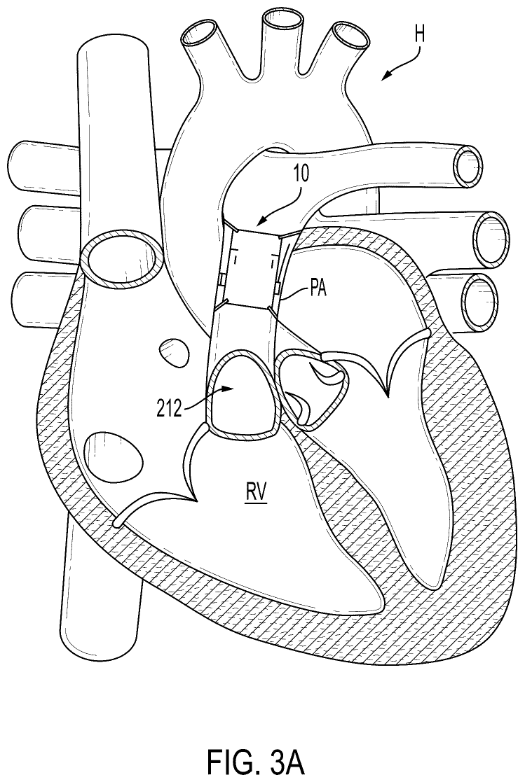

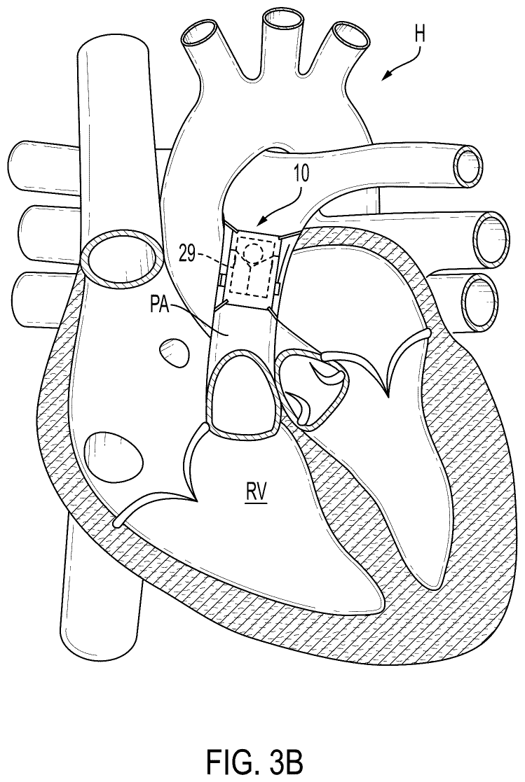

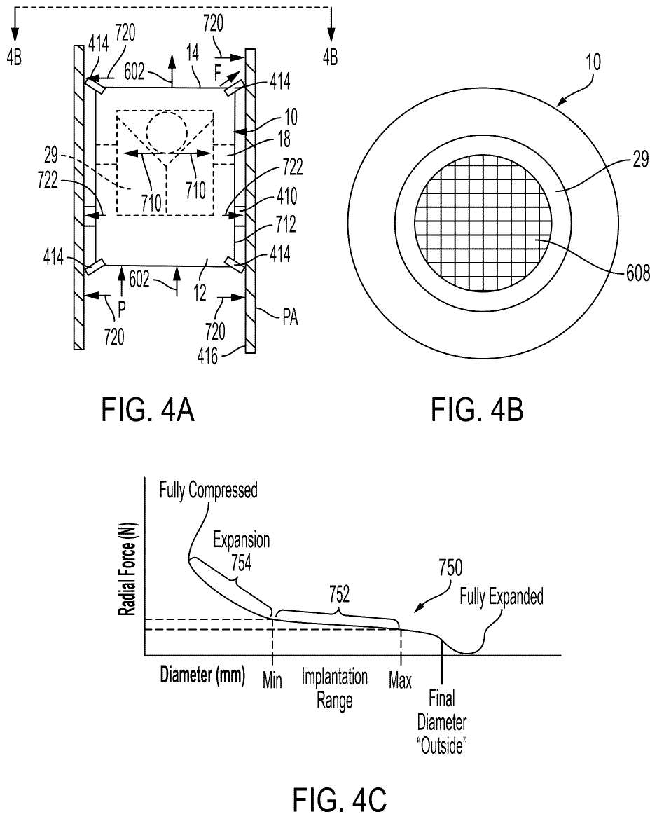

Referring to FIG. 3A, a docking station 10 is deployed in the pulmonary artery PA of a heart H. FIG. 3B illustrates a valve 29 deployed in the docking station 10 illustrated by FIG. 3A. In FIGS. 3A and 3B, the heart is in the systolic phase. FIG. 4A is an enlarged representation of the docking station 10 and valve 29 in the pulmonary artery PA of FIG. 3B. When the heart is in the systolic phase, the valve 29 opens. Blood flows from the right ventricle RV and through the pulmonary artery PA, docking station 10, and valve 29 as indicated by arrows 602. FIG. 4B illustrates a blood filled space 608 that represents the valve 29 being open when the heart is in the systolic phase. FIG. 4B does not show the interface between the docking station 10 and the pulmonary artery to simplify the drawing. The cross-hatching in FIG. 4B illustrates blood flow through the open valve. In an exemplary embodiment, blood is prevented from flowing between the pulmonary artery PA and the docking station 10 by the sealing portion(s) 410 and blood is prevented from flowing between the docking station 10 and the valve 29 by seating of the valve 29 in the seat 18 of the docking station 10. In this example, blood is substantially only flowing or only able to flow through the valve 29 when the heart is in the systolic phase.

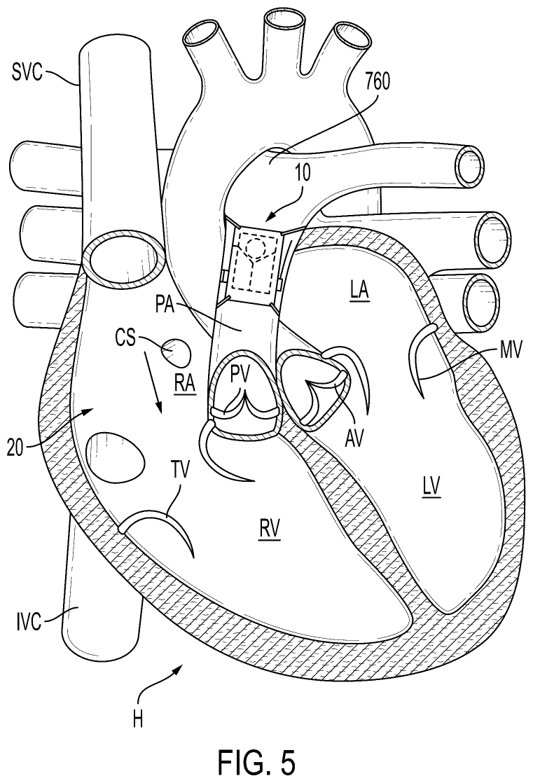

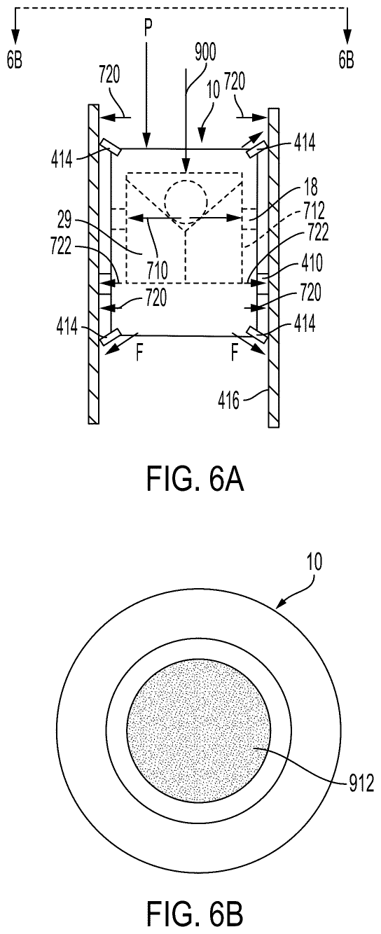

FIG. 5 illustrates the valve 29, docking station 10 and heart H illustrated by FIG. 6B, when the heart is in the diastolic phase. Referring to FIGS. 6A and 6B, when the heart is in the diastolic phase, the valve 29 closes. FIG. 6A is an enlarged representation of the docking station 10 and valve 29 in the pulmonary artery of FIG. 5. Blood flow in the pulmonary artery PA above the valve 29 (i.e. in the pulmonary branch 760) is blocked by the valve 29 being closed and blocking blood flow as indicated by arrow 900. The solid area 912 in FIG. 6B represents the valve 29 being closed when the heart is in the diastolic phase.

In one exemplary embodiment, the docking station 10 acts as an isolator that prevents or substantially prevents radial outward forces of the valve 29 from being transferred to the inner surface 416 of the circulatory system. In one embodiment, the docking station 10 includes a valve seat 18 (which is not expanded radially outwardly or is not substantially expanded radially outward by the radially outward force of the THV or valve 29, i.e., the diameter of the valve seat is not increased or is increased by less than 4 mm by the force of the THV), and anchoring/retaining portions 414 and sealing portions 410, which impart only relatively small radially outward forces 720, 722 on the inner surface 416 of the circulatory system (as compared to the radially outward force applied to the valve seat 18 by the valve 29).

When no docking station is used, stents and frames of THVs are held in place in the circulatory system by a relatively high radial outward force 710 of the stent or frame 712 of the THV acting directly on the inside surface 416 of the circulatory system. If a docking station is used, as in the example illustrated by FIG. 4A, the stent or frame 712 of the valve 29 expands radially outward or is expanded radially outward to impart the high force 710 on the valve seat 18 of the docking station 10. This high radially outward force 710 secures the valve 29 to the valve seat 18 of the docking station 10. However, since the valve seat 18 is not expanded or is not substantially expanded by the force 710, the force 710 is isolated from the circulatory system, rather than being used to secure the docking station in the circulatory system.

In an exemplary embodiment, the radially outward force 722 of the sealing portions 410 to the inside surface 416 is substantially smaller than the radially outward force 710 applied by the valve 29 to the valve seat 18. For example, the radially outward sealing force 722 can be less than ½ the radially outward force 710 applied by the valve, less than ⅓ the radially outward force 710 applied by the valve, less than ¼ the radially outward force 710 applied by the valve, less than ⅛, or even less than 1/10 the radially outward force 710 applied by the valve. In one exemplary embodiment, the radially outward force 722 of the sealing portions 410 is selected to provide a seal between the inner surface 416 and the sealing portion 410, but is not sufficient by itself to retain the position of the valve 29 and docking station 10 in the circulatory system.

In an exemplary embodiment, the radially outward force 720 of the anchoring/retaining portions 414 to the inside surface 416 is substantially smaller than the radially outward force 710 applied by the valve 29 to the valve seat 18. For example, the radially outward sealing force 720 can be less than ½ the radially outward force 710 applied by the valve, less than ⅓ the radially outward force 710 applied by the valve, less than ¼ the radially outward force 710 applied by the valve, less than ⅛, or even less than 1/10 the radially outward force 710 applied by the valve.

In one exemplary embodiment, the radially outward force 720 of the retaining portions 414 is not sufficient by itself to retain the position of the valve 29 and docking station 10 in the circulatory system. Rather, the pressure of the blood 608 is used to enhance the retention of the retaining portions 414 to the inside surface 416. Referring again to FIG. 3A, when the heart is in the systolic phase, the valve 29 is open and blood flows through the valve as indicated by arrows 602. Since the valve 29 is open and blood flows through the valve 29, the pressure P applied to the docking station 10 and valve 29 by the blood is low as indicated by the small P and arrow in FIG. 4A. Even though small, the pressure P forces the docking station and its upper retaining portions 414 against the surface 416 generally in the direction indicated by arrow F. This blood flow assisted force F applied by the retaining portions F to the surface 416 prevents the docking station 10 and valve 29 from moving in the direction 602 of blood flow in the systolic phase of the heart H.

Referring to FIG. 6A, when the heart is in the diastolic phase, the valve 29 is closed and blood flow is blocked as indicated by arrow 900. Since the valve 29 is closed and the valve 29 and docking station 10 block the flow of blood, the pressure P applied to the docking station 10 and valve 29 by the blood is high as indicated by the large arrow P in FIG. 6A. This large pressure P forces the lower retaining portions 414 against the surface 416 generally in the direction indicated by the large arrows F. This blood flow assisted force F applied by the retaining portions F to the surface 416 prevents the docking station 10 and valve 29 from moving in the direction indicated by arrow 900.

Since the force applied by the upper and lower retaining portions 414 is determined by amount of pressure applied to the valve 29 and docking station 10 by the blood, the force applied to the surface 416 is automatically proportioned. That is, the upper retaining portions are less forcefully pressed against the surface 416 when the heart is in the systolic phase than the lower retaining portions are pressed against the surface 416 when the heart is in the diastolic phase. This is because the pressure against the open valve 29 and docking station 10 in the systolic phase is less than the pressure against the closed valve and docking station in the diastolic phase.

The valve seat 18 and sealing portion 410 can take a wide variety of different forms. For example, the valve seat 18 can be any structure that is not expanded radially outwardly or is not substantially expanded radially outward by the radially outward force of the THV (i.e., the diameter of the valve seat in the deployed position/configuration may not expand or may expand less than 4 mm, e.g., the diameter may only expand 1-4 mm larger when the valve is deployed in the valve seat). For example, the valve seat 18 can comprise a suture or a metal ring that resists or limits expansion. However, in one embodiment, the valve seat 18 (or any valve seat described herein) can be expandable over a larger range, for example, the diameter may expand between 5 mm and 30 mm larger when a valve is deployed in the valve seat. In one embodiment, the diameter might expand from 5 mm or 6 mm in diameter to 20 mm-29mm, 24 mm, 26 mm, 29 mm, etc. in diameter, or expand from and to different diameters within that range. Even if more expandable, the valve seat can still be restricted in expansion, e.g., restricted to avoid expansion of the valve seat beyond an expanded diameter of a valve to be placed in the valve seat or to avoid expansion beyond a diameter that will securely hold the valve in the valve seat via the forces created therebetween. The valve seat 18 can be part of or define a portion of the body of the docking station 10, or the valve seat 18 can be a separate component that is attached to the body of the docking station. The valve seat 18 can be longer, shorter, or the same length as the valve. The valve seat 18 can be significantly shorter than the valve 29 when the valve seat 18 is defined by a suture or a metal ring. A valve seat 18 formed by a suture or metal ring can form a narrow circumferential seal line between the valve 29 and the docking station.

The sealing portion(s) 410 of various embodiments can take a wide variety of different forms. For example, the sealing portion(s) 410 can be any structure that provides a seal(s) between the docking station 10 and the surface 416 of the circulatory system. For example, the sealing portion(s) 410 can comprise a fabric, a foam, biocompatible tissue, an expandable metal frame, a combination of these, etc. The sealing portion(s) 410 can be part of or define a portion of the body of the docking station 10, and/or the sealing portion(s) 410 can be a separate component that is attached to the body of the docking station. The docking station 10 can include a single sealing portion 410 or two, or more than two sealing portions.

As mentioned above, in one exemplary embodiment the sealing portion(s) 410 is configured to apply a low radially outward force to the surface 416. The low radially outward force can be provided in a wide variety of different ways. For example, sealing portion can be made from a very compressible or compliant material. Referring to FIG. 4C, in one exemplary embodiment, the docking station 10 body is made from an elastic or super elastic metal. One such metal is nitinol. When the body of a docking station 10 is made from a lattice of metal struts, the body can have the characteristics of a spring. Referring to FIG. 4C, like a spring, when the body of the docking station is unconstrained and allowed to relax to its largest diameter the body of the docking station applies little or no radially outward force. As the body of the docking station 10 is compressed, like a spring, the radially outward force applied by the docking station increases. As is illustrated by FIG. 4C, in one exemplary embodiment the relationship of the radially outward force of the docking station body to the expanded diameter of the docking station is non-linear, although, in one exemplary embodiment, the relationship could also be linear. In the example illustrated by FIG. 4C, the curve 750 illustrates the relationship between the radially outward force exerted by the docking station 10 and the compressed diameter of the docking station. In the region 752, the curve 750 has a low slope. In this region 752 the radially outward force is low and changes only a small amount. In one exemplary embodiment, the region 752 corresponds to a diameter between 25 mm and 40 mm, such as between 27 mm and 38 mm. The radially outward force is small in the region 752, but is not zero. In the region 754, the curve 750 has a higher slope. In this region 754 the radially outward force increases significantly as the docking station is compressed. In one exemplary embodiment, the body of the stent is constructed to be in the low slope region 752. This allows the sealing portions 410 to apply only a small radially outward force to the inner surface 416 of the circulatory system over a wide range of diameters.

The retaining portions 414 can take a wide variety of different forms. For example, the retaining portion(s) 414 can be any structure that sets the position of the docking station 10 in the circulatory system. For example, the retaining portion(s) 414 can press against or into the inside surface 416 or extend around anatomical structure of the circulatory system to set the position of the docking station 10. The retaining portion(s) 414 can be part of or define a portion of the body of the docking station 10 or the retaining portion(s) 414 can be a separate component that is attached to the body of the docking station. The docking station 10 can include a single retaining portion 414 or two, or more than two retaining portions.

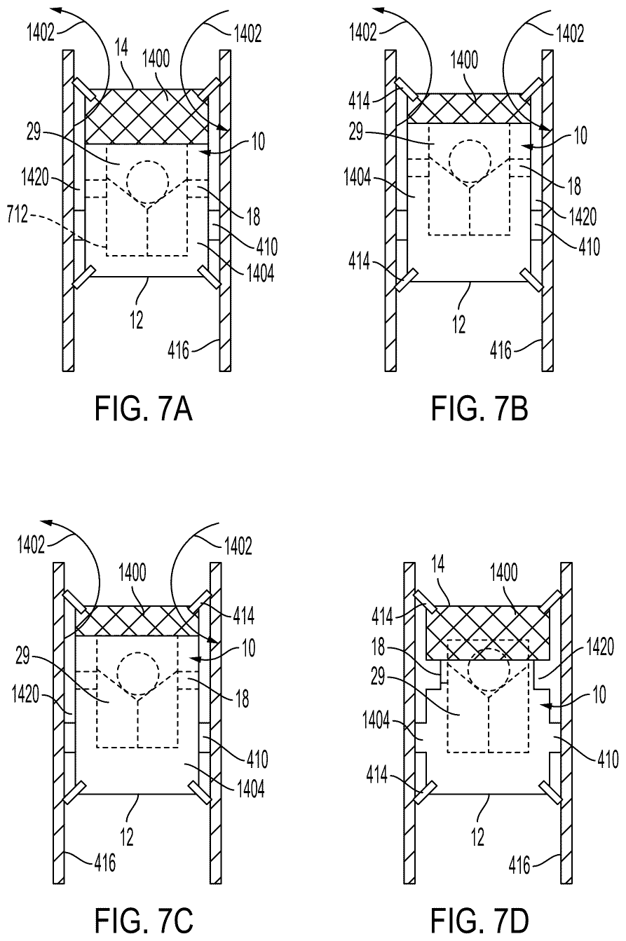

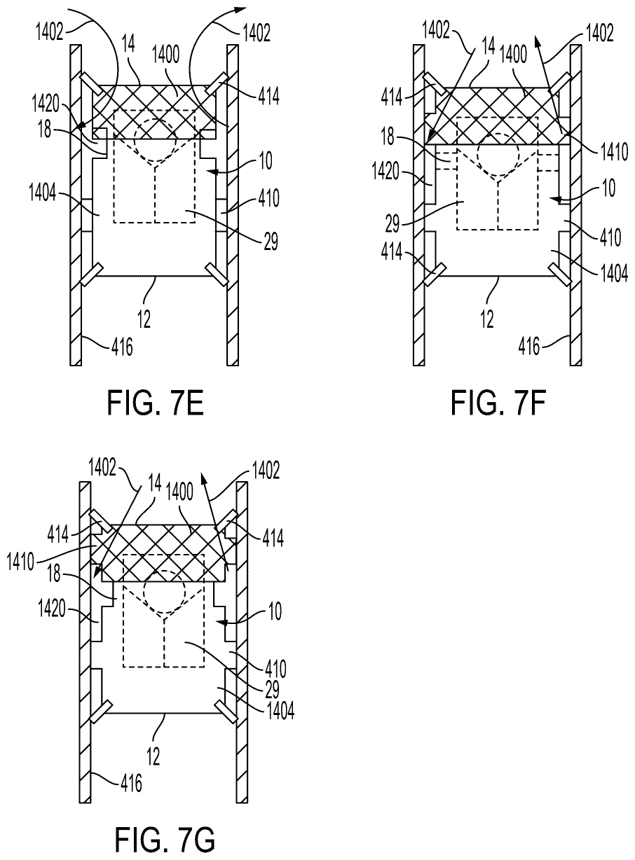

Referring to FIGS. 7A-7G, in one exemplary embodiment the docking station 10 can include a permeable portion 1400 that blood can flow through as indicated by arrows 1402 and an impermeable portion 1404 that blood cannot flow through. In one exemplary embodiment, the impermeable portion 1404 extends from at least the sealing portion 410 to the valve seat 18 to prevent blood from flowing around the valve 29. In one exemplary embodiment, the permeable portion 1400 allows blood to freely flow through it, so that portions of the docking station that do not seal against the inside surface 416 of the circulatory system or seal against the valve 29 do not block the flow of blood. For example, the docking station 10 can extend into the branch of the pulmonary artery and the portion 1400 of the docking station 10 that extends into the pulmonary artery freely allows blood to flow through the docking station 10. In one exemplary embodiment, the permeable portion 1400 allows blood to freely flow through it, so that areas 1420 between the docking station and the circulatory system are flushed with blood as the heart beats, thereby preventing blood stasis in the areas 1420.

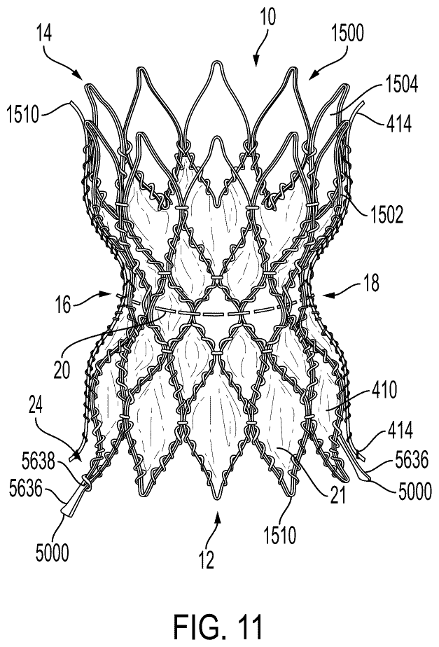

The impermeable portion 1404 can take a wide variety of different forms. The impermeable portion 1404 can be any structure or material that prevents blood to flow through the impermeable portion 1404. For example, the body of the docking station 10 can be formed from wires or a lattice, such as a nitinol wire or lattice, and cells of body are covered by an impermeable material (See FIG. 11). A wide variety of different materials can be used as the impermeable material. For example, the impermeable material can be a blood-impermeable cloth, such as a PET cloth or biocompatible covering material such as a fabric that is treated with a coating that is impermeable to blood, polyester, or a processed biological material, such as pericardium.

FIGS. 7A-7G illustrate that a wide variety of docking station configurations can be provided with a permeable portion 1400. The sealing portion 410 can be integrally formed with the body of the docking station as illustrated by FIGS. 7B, 7D, and 7F or separate as illustrated by FIGS. 7C, 7E and 7G. In FIGS. 7F and 7G the docking station 10 includes portions 1410. These portions 1410 are similar to the sealing portions 410, but a seal is not formed with the inner surface 416 of the circulatory system, because the portion 1410 is part of the permeable portion 1400. The valve seat 18 can be separately formed from the body of the docking station as illustrated by FIGS. 7A-7C or integrally formed with the body of the docking station 10 as illustrated by FIGS. 7D-7G.

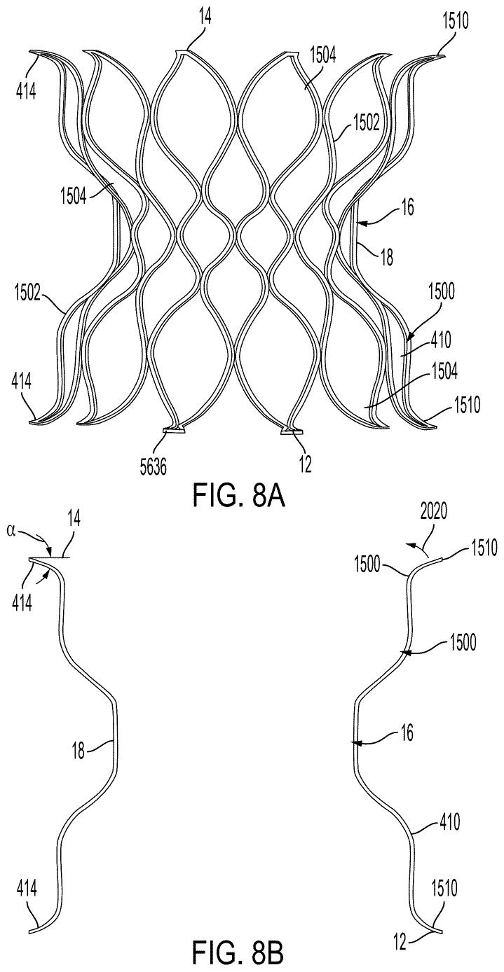

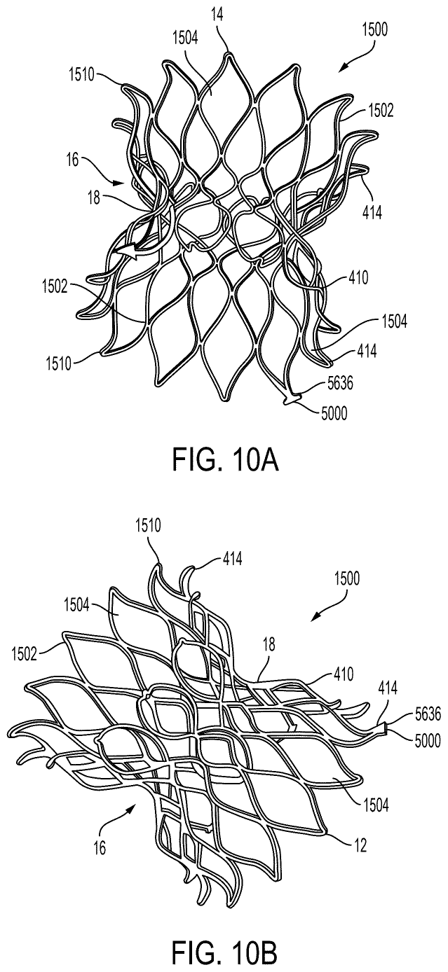

FIGS. 8A, 8B, 9, 10A, and 10B illustrate an exemplary embodiment of a frame 1500 or body of a docking station 10. The frame 1500 or body can take a wide variety of different forms and FIGS. 8A, 8B, 9, 10A, and 10B illustrate just one of the many possible configurations. In the example illustrated by FIGS. 8A, 8B, 9, 10A, 10B, and 11, the docking station 10 has a relatively wider proximal inflow end 12 and distal outflow end 14, and a relatively narrower portion 16 that forms the seat 18 in between the ends 12, 14. In the example illustrated by FIGS. 8A, 8B, 10A, and 10B, the frame 1500 of the docking station 10 is preferably a wide stent comprised of a plurality of metal struts 1502 that form cells 1504. In the example of FIGS. 8A, 8B, 10A, and 10B, the frame 1500 has a generally hourglass-shape that has a narrow portion 16, which forms the valve seat 18 when covered by an impermeable material, in between the proximal and distal ends 12, 14. As described below, the valve 29 expands in the narrow portion 16, which forms the valve seat 18.

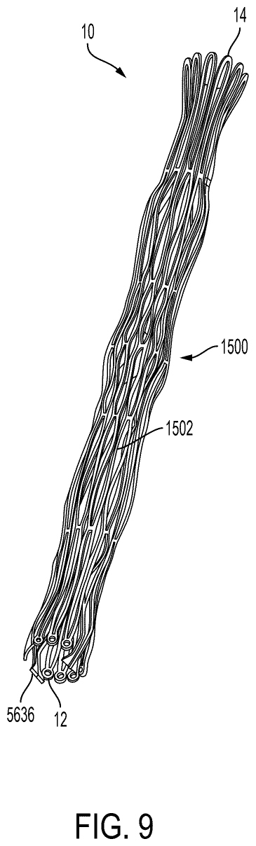

FIGS. 8A, 8B, 10A, and 10B illustrate the frame 1500 in its unconstrained, expanded condition. In this exemplary embodiment, the retaining portions 414 comprise ends or apices 1510 of the metal struts 1502 at the proximal and distal ends 12, 14. The sealing portion 410 is between the retaining portions 414 and the waist 16. In the unconstrained condition, the retaining portions 414 extend generally radially outward and are radially outward of the sealing portion 410. FIG. 9 illustrates the frame in the compressed state for delivery and expansion by a catheter. The docking station can be made from a very resilient or compliant material to accommodate large variations in the anatomy. For example, the docking station can be made from a highly flexible metal, metal alloy, polymer, or an open cell foam. An example of a highly resilient metal is nitinol, but other metals and highly resilient or compliant non-metal materials can be used. The docking station 10 can be self-expanding, manually expandable (e.g., expandable via balloon), or mechanically expandable. A self-expanding docking station 10 can be made of a shape memory material such as, for example, nitinol.

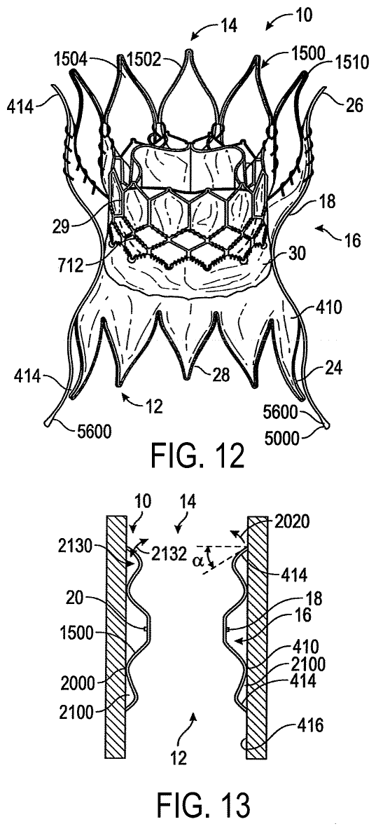

FIG. 11 illustrates the frame 1500 with impermeable material 21 attached to the frame 1500 to form the docking station 10. Referring to FIG. 11, in one exemplary embodiment a band 20 extends about the waist or narrow portion 16, or is integral to the waist to form an unexpandable or substantially unexpandable valve seat 18. The band 20 stiffens the waist and, once the docking station is deployed and expanded, makes the waist/valve seat relatively unexpandable in its deployed configuration. In the example illustrated by FIG. 12, the valve 29 is secured by expansion of its collapsible frame into the narrow portion 16, which forms the valve seat 18, of the docking station 10. As is explained above, the unexpandable or substantially unexpandable valve seat 18 prevents the radially outward force of the valve 29 from being transferred to the inside surface 416 of the circulatory system. However in another exemplary embodiment, the waist/valve seat of the deployed docking station can optionally expand slightly in an elastic fashion when the valve is deployed against it. This optional elastic expansion of the waist/valve seat 18 can put pressure on the valve 29 to help hold the valve 29 in place within the docking station.

The band can take a wide variety of different forms and can be made from a wide variety of different materials. The band 20 can be made of PET, one or more sutures, fabric, metal, polymer, a biocompatible tape, or other relatively unexpandable materials known in the art that are sufficient to maintain the shape of the valve seat 18 and hold the valve 29 in place. The band can extend about the exterior of the stent, or can be an integral part of it, such as when fabric or another material is interwoven into or through cells of the stent. The band 20 can be narrow, such as the suture band in FIG. 11, or can be wider. The band can be a variety of widths, lengths, and thicknesses. In one non-limiting example, the valve seat 18 is between 27-28 mm wide, although the diameter of the valve seat should be within the operating range of the particular valve 29 that will be secured within the valve seat 18, and can be different than the foregoing example. The valve 29, when docked within the docking station, can optionally expand around either side of the valve seat slightly. This aspect, sometimes referred to as a “dog bone” (e.g., because of the shape it forms around the valve seat or band), can also help hold the valve in place.

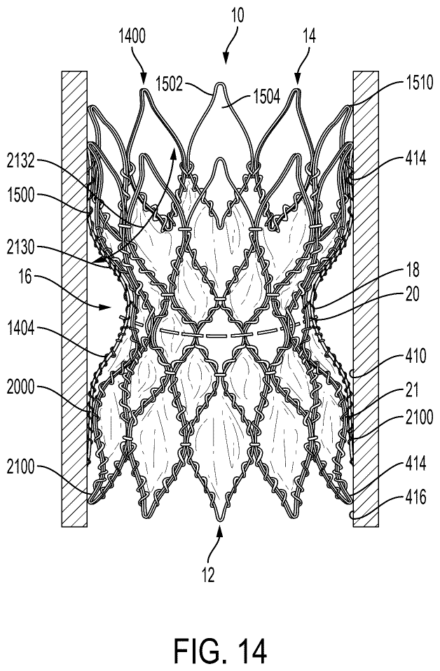

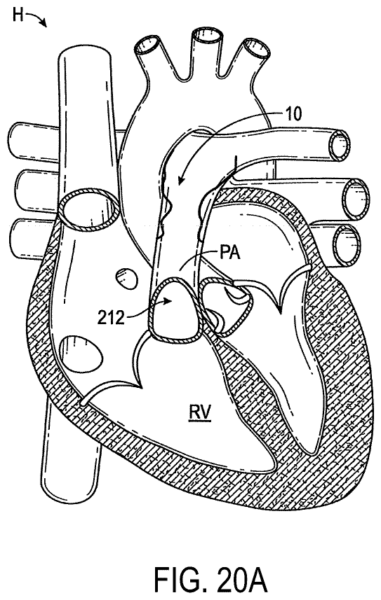

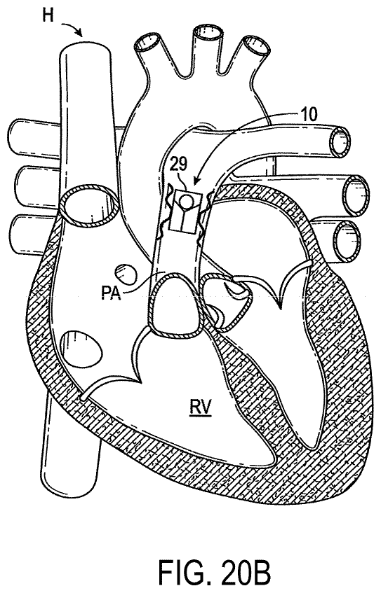

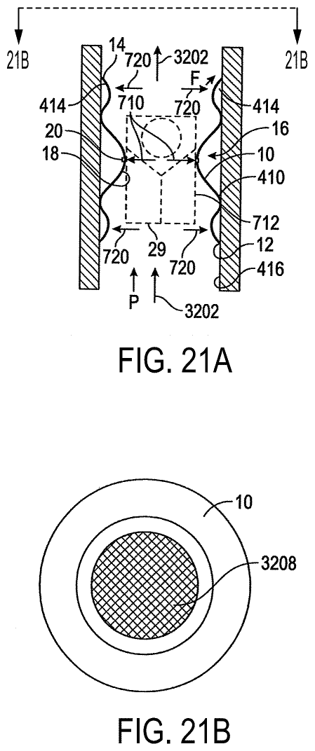

FIGS. 20A, 20B, 21A and 21B illustrate the docking station 10 of FIG. 18 implanted in the circulatory system, such as in the pulmonary artery. The sealing portions 410 provide a seal between the docking station 10 and an interior surface 416 of the circulatory system. In the example of FIGS. 13 and 14, the sealing portion 410 is formed by providing an impermeable material 21 (see FIG. 14) over the frame 1500 or a portion thereof, in particular, the sealing portion 410 can comprise the lower, rounded, radially outward extending portion 2000 of the frame 1500. In an exemplary embodiment, the impermeable material 21 extends from at least the portion 2000 of the frame 1500 to the valve seat 18. This makes the docking station impermeable from the sealing portion 410 to the valve seat 18. As such, all blood flowing in the direction of the inflow end 12 toward the outflow end 14 is directed to the valve seat 18 (and valve 29 once installed or deployed in the valve seat).

In a preferred embodiment of a docking station 10, the inflow portion has walls that are impermeable to blood, but the outflow portion walls are relatively open. In one approach, the inflow end portion 12, the mid-section 16, and a portion of the outflow end portion 14 are covered with a blood-impermeable fabric 21, which can be sewn onto the stent or otherwise attached by a method known in the art. The impermeability of the inflow portion of the stent helps to funnel blood into the docking station 10 and ultimately flow through the valve that is to be expanded and secured within the docking station 10.

From another perspective, this embodiment of a docking station is designed to seal at the proximal inflow section 2000 to create a conduit for blood flow. The distal outflow section, however, is generally left open, thereby allowing the docking station 10 to be placed higher in the pulmonary artery without restricting blood flow. For example, the permeable portion 1400 can extend into the branch of the pulmonary artery and not impede or not significantly impede the flow of blood past the branch. In one embodiment, blood-impermeable cloth, such as a PET cloth for example, or other material covers the proximal inflow section, but the covering does not cover any or at least does not cover a portion of the distal outflow section 14. As one non-limiting example, when the docking station 10 is placed in the pulmonary artery, which is a large vessel, the significant volume of blood flowing through the artery is funneled into the valve 29 by the impermeable material 21. The cloth 21 is fluid impermeable so that blood cannot pass through. Again, a variety of other biocompatible covering materials can be used such as, for example, foam or a fabric that is treated with a coating that is impermeable to blood, polyester, or a processed biological material, such as pericardium.

In the example illustrated by FIG. 14, more of the docking station frame 1500 is provided with the impermeable material 21, forming a relatively large impermeable portion 1404. In the example illustrated by FIG. 14, the impermeable portion 1404 extends from the inflow end 12 and stops one row of cells 1504 before the outflow end. As such, the most distal row of cells 1504 form a permeable portion 1400. However, more rows of cells 1504 can be uncovered by the impermeable material to form a larger permeable portion. The permeable portion 1400 allows blood to flow into and out of the area 2130 as indicated by arrows 2132. That is, blood can flow into and out of the areas 2100 in one exemplary embodiment.

The valve seat 18 can provide a supporting surface for implanting or deploying a valve 29 in the docking station 10. The retaining portions 414 can retain the docking station 10 at the implantation position or deployment site in the circulatory system. The illustrated retaining portions have an outwardly curving flare that helps secure the docking station 10 within the artery. “Outwardly” as used herein means extending away from the central longitudinal axis 5002 of the docking station. As can be seen in FIG. 13, when the docking station 10 is compressed by the inside surface 416, the retaining portions 414 engage the surface 416 at an angle α (normal to the surface to the tangent of the midpoint of the surface of the retaining portion 414) that can be between 30 and 60 degrees, such as about 45 degrees, rather than extending substantially radially outward (i.e. α is 0 to 20 degrees or about 10 degrees) as in the uncompressed condition (see FIG. 8B). This inward bending of the retaining portions 414 as indicated by arrow 2020 acts to retain the docking station 10 in the circulatory system. The retaining portions 414 are at the wider inflow end portion 12 and outflow end portion 14 and press against the inner surface 416. The flared retaining portions 414 engage into the surrounding anatomy in the circulatory system, such as the pulmonic space. In one exemplary embodiment, the flares serve as a stop, which locks the device in place. When an axial force is applied to the docking station 10, the flared retaining portions 414 are pushed by the force into the surrounding tissue to resist migration of the stent as described in more detail below. In a specific embodiment, the docking station generally has an hourglass shape, with wider distal and proximal end portions that have the flared retaining portion and a narrow, banded waist in between the ends, into which the valve is expanded.

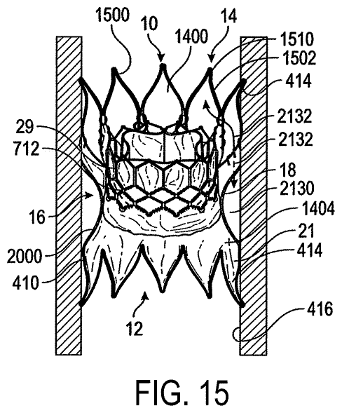

FIG. 15 illustrates the docking station 10 deployed in the circulatory system and a valve 29 deployed in the docking station 10. After the docking station 10 is deployed, the valve 29 is in a compressed form and is introduced into the valve seat 18 of the docking station 10. The valve 29 is expanded in the docking station, such that the valve 29 engages the valve seat 18. In the example illustrated by FIG. 15, the docking station 10 is longer than the valve. However, in one embodiment, the docking station 10 can be the same length or shorter than the length of the valve 29.

The valve 29 can be delivered to the site of the docking station via conventional means, such as by balloon or mechanical expansion or by self-expansion. When the valve 29 is expanded, it nests in the valve seat of the docking station 10. In one embodiment, the banded waist is slightly elastic and exerts an elastic force against the valve 29, to help hold the THV in place.

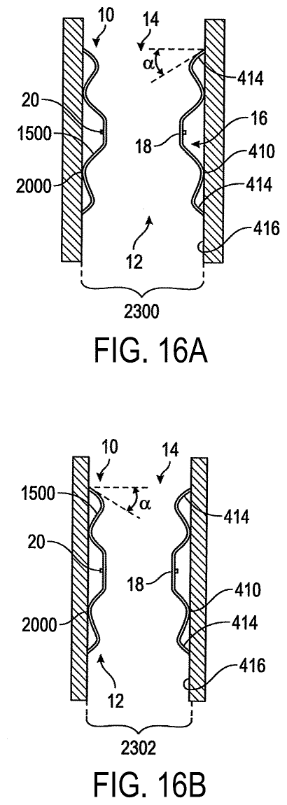

FIGS. 16A and 16B illustrate that the docking station 10 can be used to adapt a variety of different sizes of circulatory system anatomies for implantation of a valve 29 having a consistent size. In the example of FIGS. 16A and 16B, the same size docking station 10 is deployed in two different sized vessels 2300, 2302, such as two differently sized pulmonary arteries PA. In the example, the vessel 2300 illustrated by FIG. 16A has a larger effective diameter than the vessel 2302 illustrated by FIG. 16B. (Note that in this patent application the size of the anatomy of the circulatory system is referred to by the term “diameter” or “effective diameter.” The anatomy of the circulatory system is often not circular. The terms “diameter” and “effective diameter” herein refers to the diameter of a circle or disc that could be deformed to fit within the non-circular anatomy.) In the example illustrated by FIGS. 16A and 16B, the sealing portion 410 and the retaining portions 414 conform to contact each vessel 2300, 2302. However, the valve seat 18 remains the same size, even though the sealing portion 410 and the retaining portions 414 are compressed. In this manner, the docking station 10 adapts a wide variety of different anatomical sizes for implantation of a standard or single sized valve. For example, the docking station can conform to vessel diameters of 25 mm and 40 mm, such as 27 mm and 38 mm and provide a constant or substantially constant diameter valve seat of 24 mm to 30 mm, such as 27 mm to 28 mm. However, the valve seat 18 can be adapted for applications where the vessel diameter is larger or smaller than 25 mm to 40 mm and provide valve seats that are larger or smaller than 24 mm to 30 mm.

Referring to FIGS. 16A and 16B, a band 20 maintains a constant or substantially constant diameter of the valve seat 18, even as the proximal and distal ends of the docking station expand to respective diameters necessary to engage with the inside surface 416. The diameter of the pulmonary artery PA can vary considerably from patient to patient, but the valve seat 18 in the deployed configuration consistently has a diameter that is within an acceptable range for the valve 29.

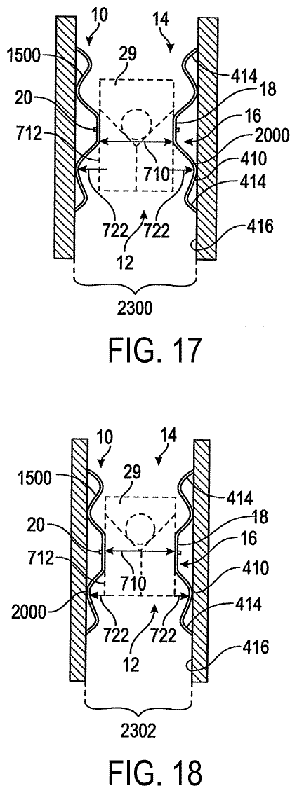

FIGS. 17 and 18 illustrate side profiles of the docking station 10 illustrated by FIG. 11 when implanted in different sized vessels 2300, 2302 of the circulatory system with a schematically illustrated transcatheter heart valve 29 having the same size installed or deployed in each docking station 10. In this example, the docking station 10 both accommodates vessels 2300, 2302 having a variety of different sizes and acts as an isolator that prevents or substantially prevents radial outward forces of the valve 29 from being transferred to the vessels. The valve seat 18 is not expanded radially outwardly or is not substantially expanded radially outward by the radially outward force of the valve 29 and the anchoring/retaining portions 414 and the sealing portions 410 impart only relatively small radially outward force on the vessels 2300, 2302 (as compared to the radially outward force applied to the valve seat 18 by the valve 29), even when the docking station is deployed in a vessel 2302 having a smaller diameter.

In the example illustrated by FIGS. 17 and 18, the stent or frame 712 of the valve 29 expands radially outward or is expanded radially outward to import the high force 710 on the valve seat 18 of the docking station 10. This high radially outward force 710 secures the valve 29 to the valve seat 18 of the docking station 10. However, since the valve seat 18 is not expanded or is not substantially expanded by the force 710, the force 710 is isolated from the circulatory system, rather than being used to secure the docking station in the circulatory system.

In an exemplary embodiment, the radially outward force 722 of the sealing portions 410 to both the larger vessel 2300 and the smaller vessel is substantially smaller than the radially outward force 710 applied by the valve 29 to the valve seat 18. For example, for the smallest vessel to be adapted by the docking station 10 for valve implantation, the radially outward sealing force 722 can be less than 1/2 the radially outward force 710 applied by the valve, less than ⅓ the radially outward force 710 applied by the valve, less than ¼ the radially outward force 710 applied by the valve, less than ⅛, or even less than 1/10 the radially outward force 710 applied by the valve. In one exemplary embodiment, the radially outward force 722 of the sealing portions 410 is selected to provide a seal between the inner surface 416 and the sealing portion 410, but is not sufficient by itself to retain the position of the valve 29 and docking station 10 in the circulatory system. In one embodiment, the radially outward force 722 is sufficient to retain the position of the valve 29 and docking station 10 in the circulatory system.

In an exemplary embodiment, the docking station 10 illustrated by FIG. 11 also includes anchoring/retaining portions 414 that apply radially outward forces 720 that are substantially smaller than the radially outward force 710 applied by the valve 29 to the valve seat 18. For example, for the smallest vessel to be adapted by the docking station 10 for valve implantation, the radially outward sealing force 720 can be less than 1/2 the radially outward force 710 applied by the valve, less than ⅓ the radially outward force 710 applied by the valve, less than ¼ the radially outward force 710 applied by the valve, less than ⅛, or even less than 1/10 the radially outward force 710 applied by the valve. In one embodiment, the radially outward force 720 of the anchoring/retaining portions 414 is not sufficient by itself to retain the position of the valve 29 and docking station 10 in the circulatory system. In one embodiment, the radially outward force 720 is sufficient to retain the position of the valve 29 and docking station 10 in the circulatory system.

In one exemplary embodiment, the docking station 10 frame 1500 is made from an elastic or superelastic material or metal. One such metal is nitinol. When the frame 1500 of the docking station 10 is made from a lattice of metal struts, the body can have the characteristics of a spring. Referring to FIG. 4C, like a spring, when the frame 1500 of the docking station 10 illustrated by FIGS. 17 and 18 is unconstrained and allowed to relax to its largest diameter the frame of the docking station applies little or no radially outward force. As the frame 1500 of the docking station 10 is compressed, like a spring, the radially outward force applied by the docking station increases. As is illustrated by FIG. 4C, in one exemplary embodiment the relationship of the radially outward force of the docking station frame 1500 to the expanded diameter of the docking station is non-linear, though it can also be linear. In the example illustrated by FIG. 4C, the curve 750 illustrates the relationship between the radially outward force exerted by the docking station 10 and the compressed diameter of the docking station. In the region 752, the curve 750 has a low slope. In this region 752 the radially outward force is low and changes only a small amount. In one exemplary embodiment, the region 752 corresponds to a diameter between 25 mm and 40 mm, such as between 27 mm and 38 mm. The radially outward force is small in the region 752, but is not zero. In the region 754, the curve 750 has a higher slope. In this region 754 the radially outward force increases significantly as the docking station is compressed. In one exemplary embodiment, the body of the stent is constructed to be in the low slope region 752 for both a largest vessel 2300 (FIG. 17) accommodated by the docking station 10 and a smallest vessel 2302 (FIG. 18). This allows the sealing portions 410 to apply only a small radially outward force to the inner surface 416 of the circulatory system over a wide range of diameters.

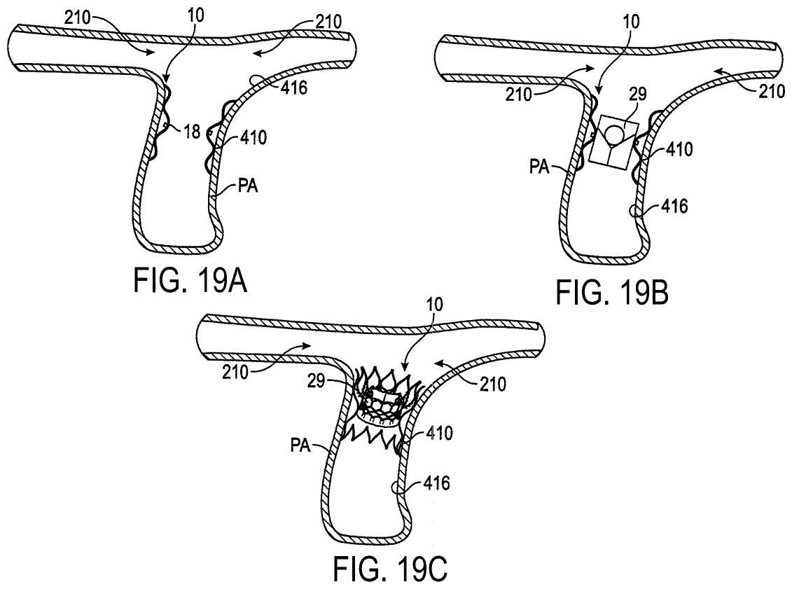

FIGS. 19A-19C illustrate the docking station 10 of FIG. 11 implanted in a pulmonary artery. FIG. 19A illustrates the profile of the docking station 10 implanted in the pulmonary artery PA. FIG. 19B illustrates the profile of the docking station 10 implanted in the pulmonary artery PA with a schematically illustrated valve 29 installed or deployed in the docking station 10. FIG. 19C illustrates the docking station 10 and valve 29 as depicted in FIG. 15 implanted in the pulmonary artery PA. As mentioned with respect to FIGS. 2A and 2B, the shape of the pulmonary artery may vary significantly along its length. In one exemplary embodiment, the docking station 10 is configured to conform to the varying shape of the pulmonary artery PA. The docking station 10 is illustrated as being positioned below the pulmonary artery bifurcation or branch. However, often the docking station 10 will be positioned such that the end 14 extends into the pulmonary artery bifurcation 210. When it is contemplated that the docking station 10 will extend into the pulmonary artery bifurcation, the docking station 10 can have a blood permeable portion 1400 (e.g., as shown in FIG. 14).

Referring to FIG. 20A, the docking station illustrated by FIG. 11 is deployed in the pulmonary artery PA of a heart H. FIG. 20B illustrates a generically illustrated valve 29 deployed in the docking station 10 illustrated by FIG. 20A. In FIGS. 20A and 20B, the heart is in the systolic phase. FIG. 21A is an enlarged representation of the docking station 10 and valve 29 in the pulmonary artery PA of FIG. 20B. When the heart is in the systolic phase, the valve 29 opens. Blood flows from the right ventricle RV and through the pulmonary artery PA, docking station 10, and valve 29 as indicated by arrows 3202. FIG. 21B illustrates space 3208 that represents the valve 29 being open when the heart is in the systolic phase. FIG. 21B does not show the interface between the docking station 10 and the pulmonary artery to simplify the drawing. The cross-hatching in FIG. 21B illustrates blood flow through the open valve. In an exemplary embodiment, blood is prevented from flowing between the pulmonary artery PA and the docking station 10 by the sealing portion 410 and blood is prevented from flowing between the docking station 10 and the valve 29 by seating of the valve 29 in the seat 18 of the docking station 10. In this example, blood is substantially only or only able to flow through the valve 29 when the heart is in the systolic phase.

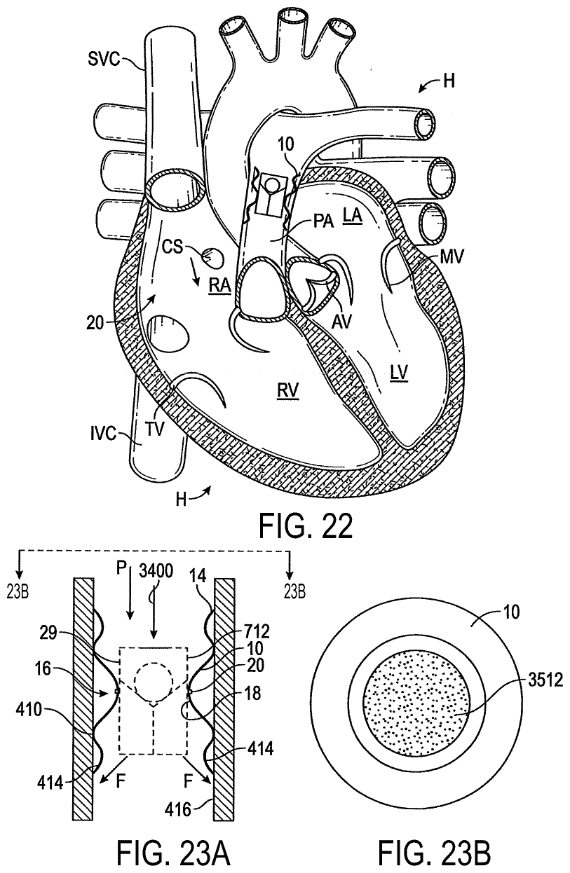

FIG. 22 illustrates the valve 29, docking station 10 and heart H illustrated by FIG. 20B, when the heart is in the diastolic phase. Referring to FIG. 22, when the heart is in the diastolic phase, the valve 29 closes. FIG. 23A is an enlarged representation of the docking station 10 and valve 29 in the pulmonary artery of FIG. 22. Blood flow in the pulmonary artery PA above the valve 29 (i.e. in the pulmonary branch 210) is blocked by the valve 29 being closed and blocking blood flow as indicated by arrow 3400. The solid area 3512 in FIG. 23B represents the valve 29 being closed when the heart is in the diastolic phase.

Referring to FIG. 21A, the radially outward force 720 of the anchoring/retaining portions 414 to the inside surface 416 is substantially smaller than the radially outward force 710 applied by the valve 29 to the valve seat 18. For example, the radially outward sealing force 720 can be less than ½ the radially outward force 710 applied by the valve, less than ⅓ the radially outward force 710 applied by the valve, less than ¼ the radially outward force 710 applied by the valve, less than ⅛, or even less than 1/10 the radially outward force 710 applied by the valve.

Referring to FIGS. 21A and 23A, in one exemplary embodiment the radially outward force 720 of the retaining portions 414 is not sufficient by itself to retain the position of the valve 29 and docking station 10 in the circulatory system. Rather, the pressure of the blood in the space 3208 is used to enhance the retention of the retaining portions 414 to the inside surface 416. Referring again to FIG. 21A, when the heart is in the systolic phase, the valve 29 is open and blood flows through the valve as indicated by arrows 3202. Since the valve 29 is open and blood flows through the valve 29, the pressure P applied to the docking station 10 and valve 29 by the blood is low as indicated by the small P and arrow in FIG. 21A. Even though small, the pressure P forces the docking station and its upper retaining portions 414 against the surface 416 generally in the direction indicated by arrow F (the small F represents a relatively low force). This blood flow assisted force F applied by the retaining portions F to the surface 416 prevents the docking station 10 and valve 29 from moving in the direction 3202 of blood flow in the systolic phase of the heart H.

Referring to FIG. 23A, when the heart is in the diastolic phase, the valve 29 is closed and blood flow is blocked as indicated by arrow 3400. Since the valve 29 is closed and the valve 29 and docking station 10 block the flow of blood, the pressure P applied to the docking station 10 and valve 29 by the blood is high as indicated by the large arrow P in FIG. 23A. This large pressure P forces the lower retaining portions 414 against the surface 416 generally in the direction indicated by the large arrows F (the large F represents a relatively larger force). This blood flow assisted force F applied by the retaining portions F to the surface 416 prevents the docking station 10 and valve 29 from moving in the direction indicated by arrow 3400.

Referring to FIGS. 21A and 23A, since the force applied by the upper and lower retaining portions 414 is determined by amount of pressure applied to the valve 29 and docking station 10 by the blood, the force applied to the surface 416 is automatically proportioned. That is, the upper retaining portions are less forcefully pressed against the surface 416 when the heart is in the systolic phase than the lower retaining portions are pressed against the surface 416 when the heart is in the diastolic phase. This is because the pressure against the open valve 29 and docking station 10 in the systolic phase is less than the pressure against the closed valve and docking station in the diastolic phase.

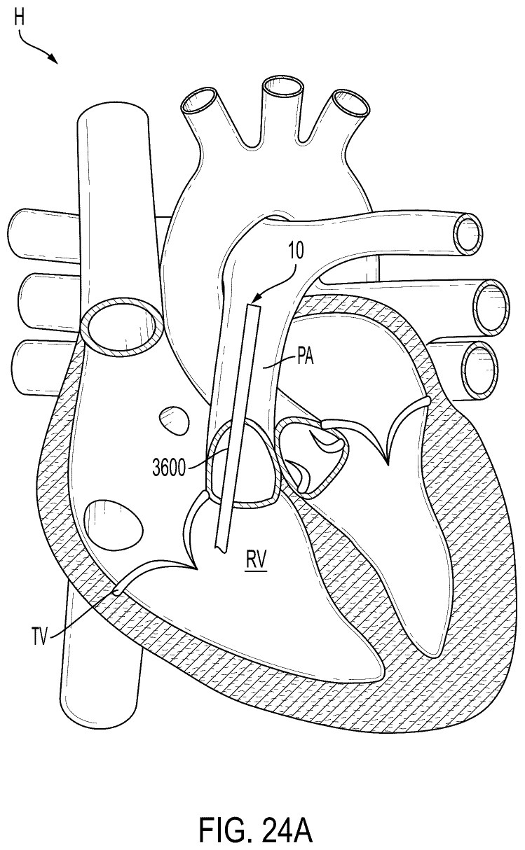

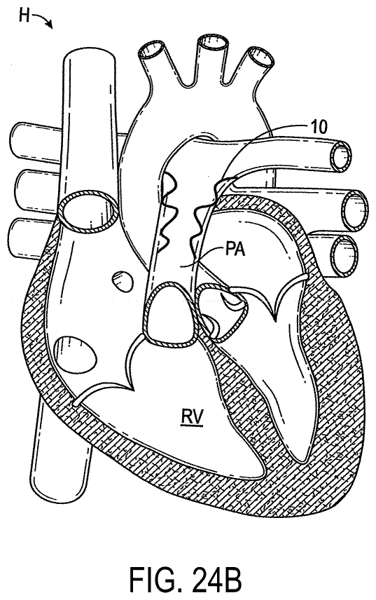

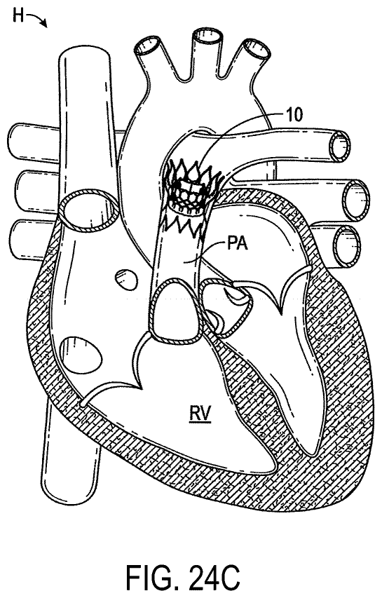

Methods of treating a subject (e.g., methods of treating heart valve dysfunction/regurgitation/etc.) can include a variety of steps, including steps associated with introducing and deploying a docking station in a desired location/treatment area and introducing and deploying a valve in the docking station. For example, FIG. 24A illustrates the docking station illustrated by FIG. 11 being deployed by a catheter 3600. The docking station 10 can be positioned and deployed in a wide variety of different ways. Access can be gained through the femoral vein or access can be percutaneous. Generally, any vascular path that leads to the pulmonary artery can be used. In one exemplary embodiment, a guidewire followed by a catheter 3600 is advanced to the pulmonary artery PA by way of the femoral vein, inferior vena cava, tricuspid valve and right ventricle RV. The docking station 10 can be placed in the right ventricular outflow tract/pulmonary artery PA to create an artificial conduit and landing zone for a valve (e.g., a transcatheter heart valve) 29.

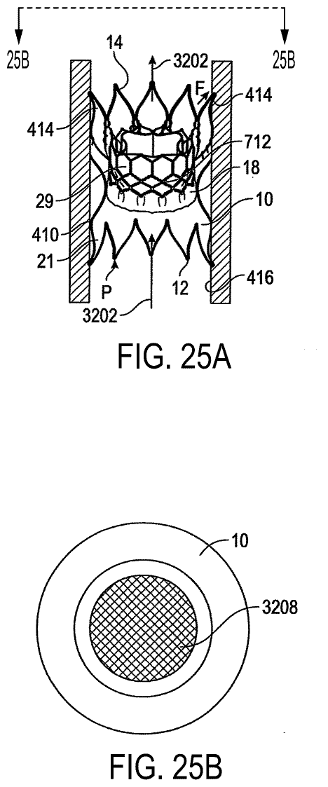

Referring to FIG. 24B, the docking station illustrated by FIG. 11 is deployed in the pulmonary artery (PA) of a heart H. FIG. 24C illustrates a valve 29 deployed in the docking station 10 illustrated by FIG. 20A. In the example illustrated by FIGS. 24C, 25A, 26, 27A, and 27B, the valve 29 is depicted as a SAPIEN 3 THV provided by Edwards Lifesciences; however, a variety of other valves can also be used. In FIGS. 24A-24C, the heart is in the systolic phase. FIG. 25A is an enlarged representation of the docking station 10 and valve 29 in the pulmonary artery of FIG. 24C. When the heart is in the systolic phase, the valve (e.g., Sapien 3 valve) is open. Blood flows from the right ventricle RV and through the pulmonary artery PA, docking station 10, and valve as indicated by arrows 3202. FIG. 25B illustrates space 3208 that represents the valve being open when the heart is in the systolic phase. FIG. 25B does not show the interface between the docking station 10 and the pulmonary artery to simplify the drawing. The cross-hatching in FIG. 25B illustrates blood flow through the valve. In an exemplary embodiment, blood is prevented from flowing between the pulmonary artery PA and the docking station 10 by the sealing portion 410 and blood is prevented from flowing between the docking station 10 and the valve by seating of the valve in the seat 18 of the docking station 10. In this example, blood is substantially only or only able to flow through the valve when the heart is in the systolic phase.

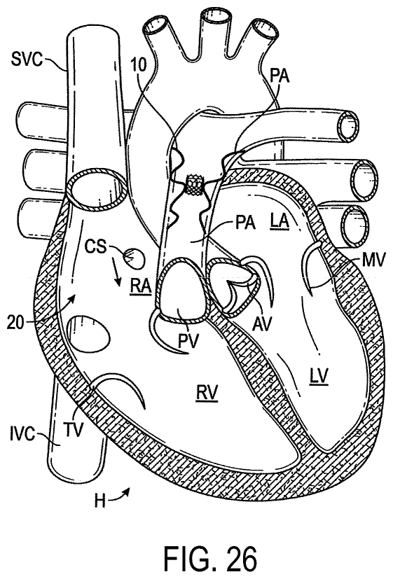

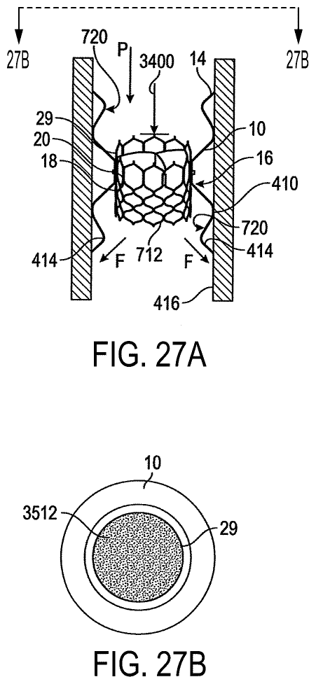

FIG. 26 illustrates the valve 29, docking station 10 and heart H illustrated by FIG. 24C, when the heart is in the diastolic phase. Referring to FIG. 26, when the heart is in the diastolic phase, the valve 29 closes. FIG. 27A is an enlarged representation of the docking station 10 and valve (e.g., Sapien 3 valve) in the pulmonary artery PA of FIG. 26. Blood flow in the pulmonary artery PA above the valve 29 (i.e. in the pulmonary branch 210) is blocked by the valve 29 being closed and blocking blood flow as indicated by arrow 3400. The solid area 3512 in FIG. 27B represents the valve 29 being closed when the heart is in the diastolic phase.

Referring to FIG. 27A, the radially outward force 720 of the anchoring/retaining portions 414 to the inside surface 416 is substantially smaller than the radially outward force 710 applied by the valve (e.g., Sapien 3 valve) to the valve seat 18. For example, the radially outward sealing force 720 can be less than ½ the radially outward force 710 applied by the valve, less than ⅓ the radially outward force 710 applied by the valve, less than ¼ the radially outward force 710 applied by the valve, less than ⅛, or even less than 1/10 the radially outward force 710 applied by the valve. The 29 mm size Sapien 3 valve typically applies radially outward force 710 of about 42 Newtons. In one embodiment, the radially outward force of deployed docking stations described herein, or one or more portions of a deployed docking stations can be between about 4 to 16 Newtons, though other forces are also possible.

The valve 29 used with the docking station 10 can take a wide variety of different forms. In one exemplary embodiment, the valve 29 is configured to be implanted via a catheter in the heart H. For example, the valve 29 can be expandable and collapsible to facilitate transcatheter application in a heart. However, in other embodiments, the valve 29 can be configured for surgical application. Similarly, the docking stations described herein can be placed using transcatheter application/placement or surgical application/placement.

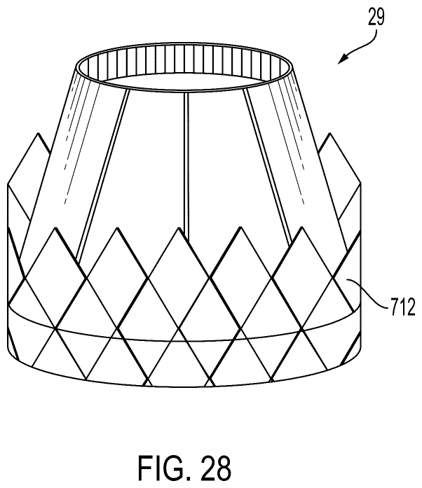

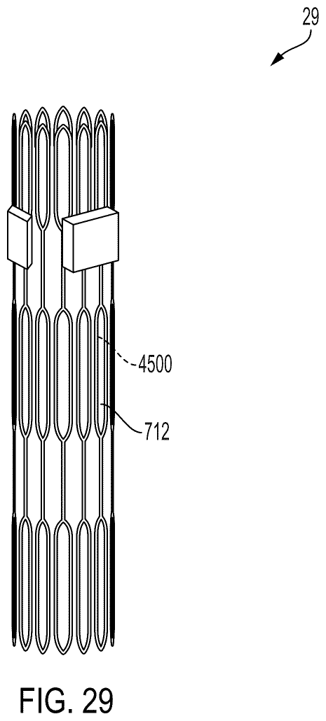

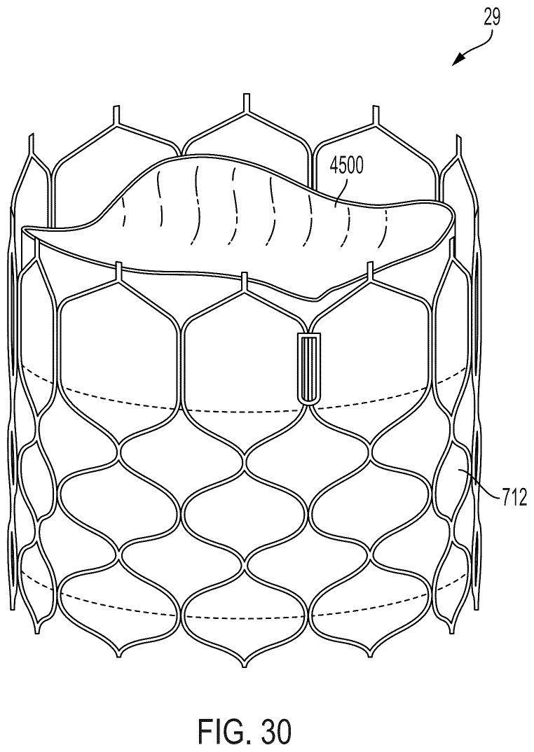

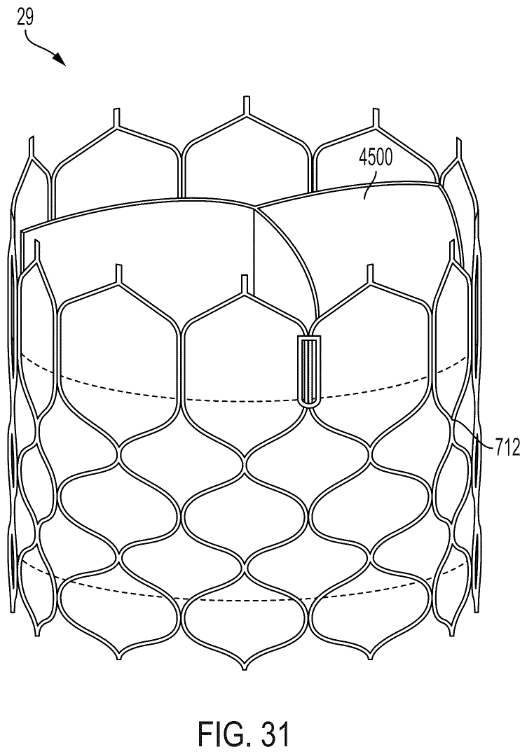

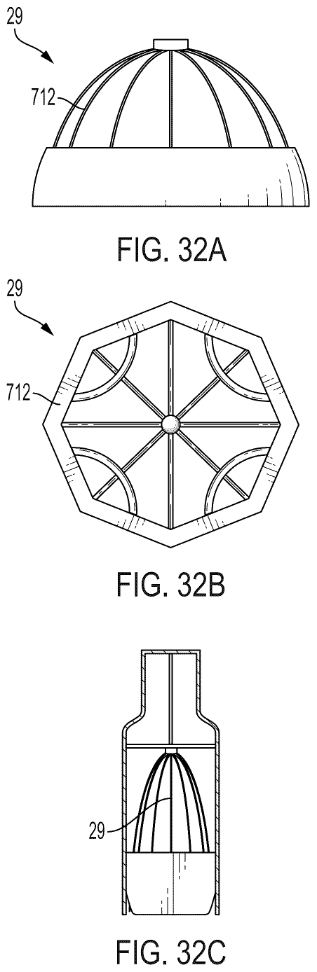

FIGS. 28-32C illustrate a few examples of the many valves or valve configurations that can be used. Any valve type can be used and some valves that are traditionally applied surgically can be modified for transcatheter implantation. FIG. 28 illustrates an expandable valve 29 for transcatheter implantation that is shown and described in U.S. Pat. No. 8,002,825, which is incorporated herein by reference in its entirety. An example of a tri-leaflet valve is shown and described in Published Patent Cooperation Treaty Application No. WO 2000/42950, which is incorporated herein by reference in its entirety. Another example of a tri-leaflet valve is shown and described in U.S. Pat. No. 5,928,281, which is incorporated herein by reference in its entirety. Another example of a tri-leaflet valve is shown and described in U.S. Pat. No. 6,558,418, which is incorporated herein by reference in its entirety. FIGS. 29-31 illustrate an exemplary embodiment of an expandable tri-leaflet valve 29, such as the Edwards SAPIEN Transcatheter Heart Valve. Referring to FIG. 29, in one exemplary embodiment the valve 29 comprises a frame 712 that contains a tri-leaflet valve 4500 (See FIG. 30) compressed inside the frame 712. FIG. 30 illustrates the frame 712 expanded and the valve 29 in an open condition. FIG. 31 illustrates the frame 712 expanded and the valve 29 in a closed condition. FIGS. 32A, 32B, and 32C illustrate an example of an expandable valve 29 that is shown and described in U.S. Pat. No. 6,540,782, which is incorporated herein by reference in its entirety. An example of a valve is shown and described in U.S. Pat. No. 3,365,728, which is incorporated herein by reference in its entirety. Another example of a valve is shown and described in U.S. Pat. No. 3,824,629, which is incorporated herein by reference in its entirety. Another example of a valve is shown and described in U.S. Pat. No. 5,814,099, which is incorporated herein by reference in its entirety. Any of these or other valves can be used as valve 29 in the various embodiments disclosed herein.

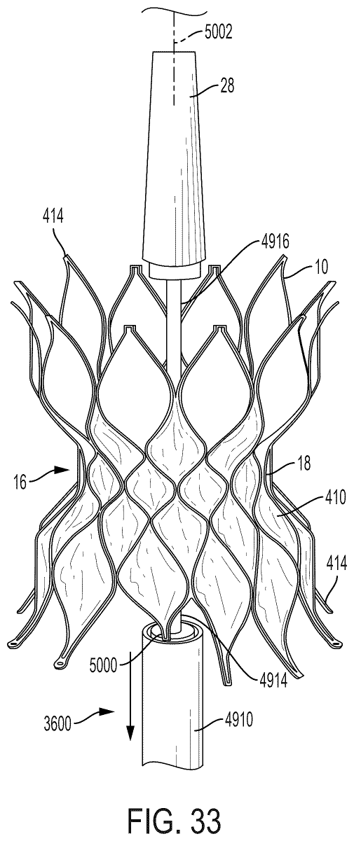

FIG. 33 illustrates a docking station 10 being deployed from a catheter 3600, with the docking station 10 expanded out of an outer tube 4910 of the catheter, with elongated legs 5000 remaining retained by a docking station connector 4914 in the outer tube 4910. During deployment of a docking station in the circulatory system, similar steps can be used, and the docking station can be deployed in a similar way.

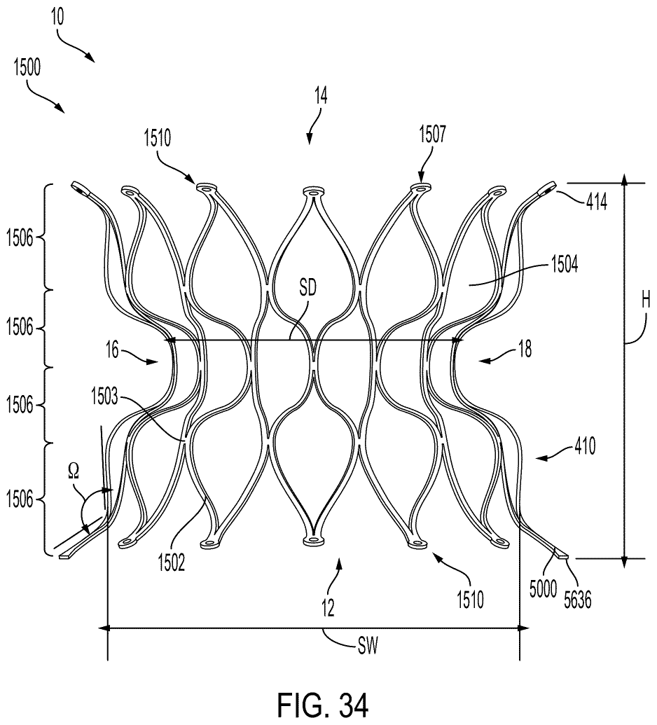

FIGS. 34 through 63 show additional embodiments of docking stations, and frames, covers, markers, and other features for docking stations. Any combination or sub-combination of features, or any individual feature of the embodiments of FIGS. 34 through 63 can be used/combined with any combination or sub-combination of features, or any individual feature of the embodiments of FIGS. 3A through 33, as well as embodiments of commonly owned U.S. Pat. No. 10,363,130 and Patent Cooperation Treaty Application No. PCT/US2017/016587, which are incorporated herein by reference in their entireties.

Referring now to FIGS. 34 through 38B, the frame 1500 of the docking station 10 can be sized, shaped, and/or otherwise configured to fit pulmonary arteries of varying sizes, shapes, diameters, and geometries. The frame 1500 of the docking station 10 can have any number of struts 1502, any number of cells 1504, or any number of apices 1510, or the struts 1502 or the cells 1504 can have any shape to fit pulmonary arteries of varying sizes, shapes, and geometries. The struts 1502 can have any size, shape, thickness, or configuration to retain the valve 29 in the pulmonary artery PA. Additionally, the proximal end 12 of the frame 1500 can have a different size, shape, and/or configuration from the distal end 14 of the frame 1500.

The frame 1500 of the docking station 10 can include a lattice of struts 1502 which extend from the proximal end 12 to the distal end 14 and define the valve seat 18. The struts 1502 each extend from the apices 1510 at the proximal end 12 to the nearest junction 1503, extend between adjacent junctions 1503, and extend from the apices 1510 at the distal end 14 to the nearest junction 1503. As such, each strut 1502 connects with one or more other struts 1502 at a junction 1503 and/or apex 1510. The space enclosed by the junctions 1503, apices 1510, and connected struts 1502 define the cells 1504. The struts 1502 can connect at the proximal and distal ends 12, 14 to form a plurality of apices 1510. The apices 1510 can serve as or connect to the retaining portions 414. Rungs 1506 are a circumferential row of the struts 1502 that extend from the apices 1510 at the proximal end 12 to the nearest junction 1503, circumferential row(s) of the struts 1502 that extend between adjacent junctions 1503, and/or a circumferential row of struts 1502 that extends from the apices 1510 at the distal end 14 to the nearest junction 1503. In the example illustrated by FIG. 34, the frame 1500 comprises four rungs. The struts 1502 alternate between converging with the junctions 1503 pointed toward the proximal end 12 and converging with the junctions 1503 pointed toward the distal end 14, such that the cells 1504 are generally diamond shaped. Additionally or alternatively, one or more struts 1502 in one rung 1506 can be continuous with one or more struts 1502 in the successive rung 1506. That is, one or more of the struts 1502 can be formed from a continuous strip of material that is simply connected to an adjacent strut at the junctions 1503, rather than each strut 1502 terminating on one side of a junction 1503 with a discrete strut starting on the other side of the junction.

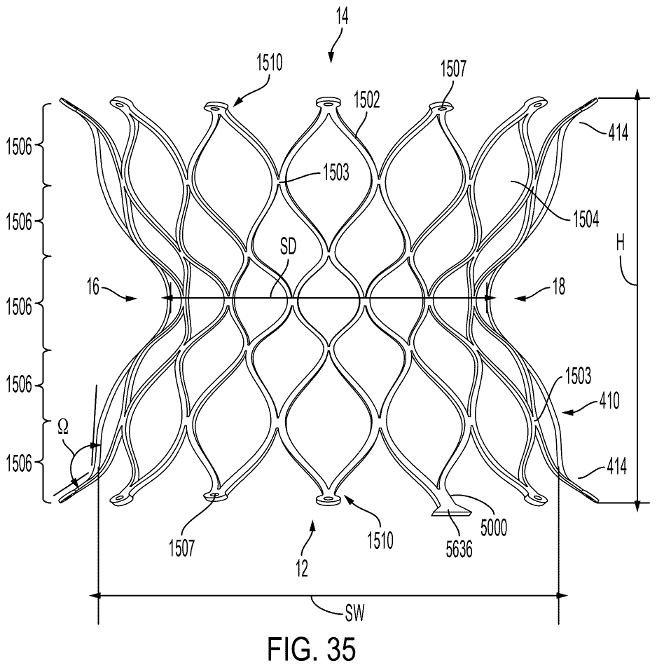

As shown in FIGS. 34 and 35, the frame 1500 can have a height H extending from the proximal end 12 to the distal end 14 of the frame and a seat diameter SD which is the diameter of the valve seat 18. The frame 1500 can also have a seal width SW which is the width of the sealing portion 410 at the point between the proximal end 12 and the valve seat 18 where the docking station 10 seals with the pulmonary artery.

Referring to FIGS. 34 and 35, the frame 1500 of the docking station 10 can have different numbers of rungs 1506. The number and configuration of rungs 1506 can be determined to provide a better securement, fit, or apposition of the docking station 10 in the pulmonary artery PA. For example, the docking station 10 can include more rungs 1506 for longer pulmonary arteries PA or where more radial force is beneficial.

As shown in FIG. 34, the frame 1500 of the docking station 10 can be configured for wide pulmonary arteries PA. For example, the frame 1500 of the docking station 10 can be configured for pulmonary arteries PA that are short and wide. The frame 1500 of the docking station 10 can have four rungs 1506 and can have three rows of cells 1504. The frame 1500 can have a height H between 30 mm and 40 mm, such as between 32 mm and 38 mm, such as 35 mm. The frame 1500 can have a seat diameter SD between 24 mm and 31 mm, such as between 26 mm and 29 mm, such as 27 mm. The frame 1500 can have a seal width SW between 36 mm and 46 mm, such as between 38 mm and 44 mm, such as 41 mm.

The frame 1500 of the docking station 10 can also be configured to fit a longer and/or wider pulmonary artery. For example, the frame 1500 of the docking station 10 can be longer and wider. As shown in FIG. 35, the frame 1500 of the docking station 10 can have six rungs 1506 and can have five rows of cells 1504. The frame 1500 of the docking station 10 can have a height H between 43 mm and 53 mm, such as between 45 mm and 51 mm, such as 48 mm. The frame 1500 can have a seat diameter SD between 24 mm and 31 mm, such as between 26 mm and 29 mm, such as 27 mm. The frame 1500 can have a seal width SW between 44 mm and 54 mm, such as between 46 mm and 52 mm, such as 48 mm and 50 mm.

While the frame 1500 has been described as having either four or six rungs 1506, the frame 1500 can have any suitable number of rungs 1506 and any suitable number of rows of cells 1504. For example, the frame 1500 can have three, five, or seven or more rungs 1506 and two, four, or six or more rows of cells 1504. The frame 1500 can also have alternative configurations or geometries such that the frame 1500 does not have diamond-shaped cells 1504 or not all the cells 1504 are diamond-shaped.

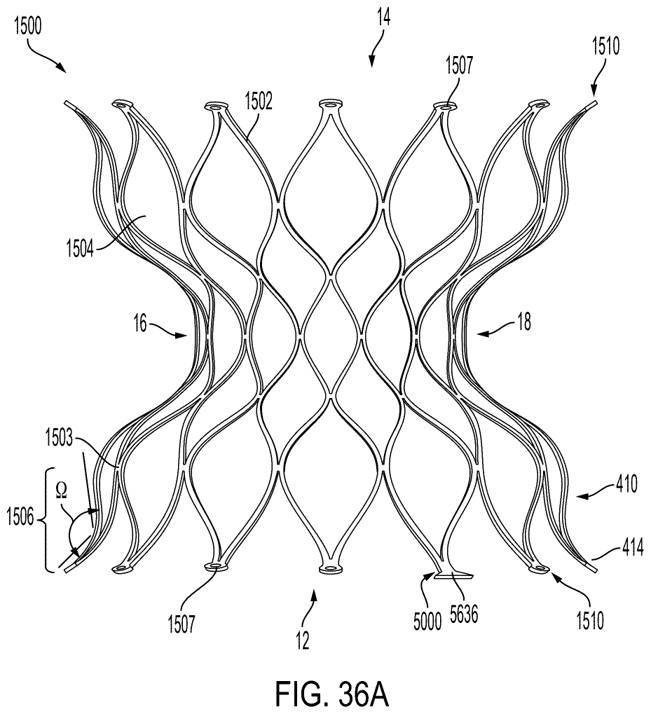

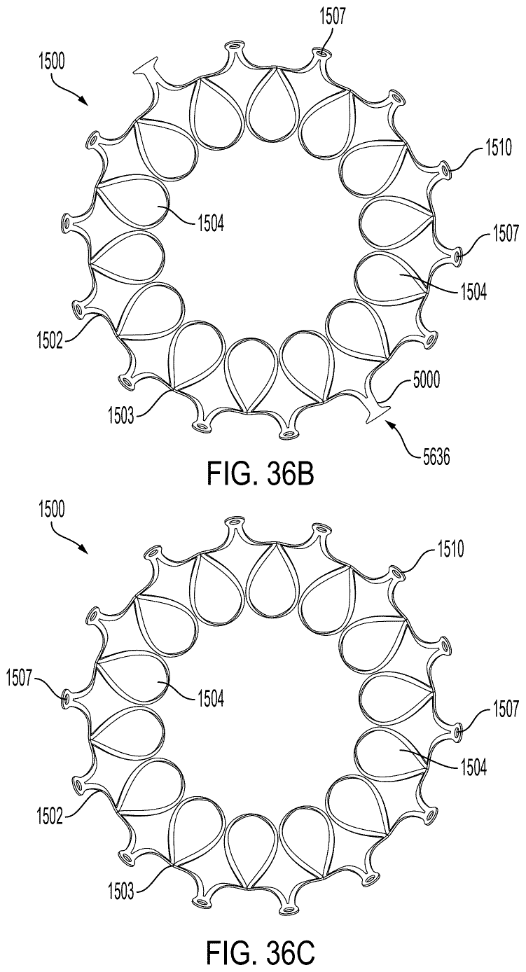

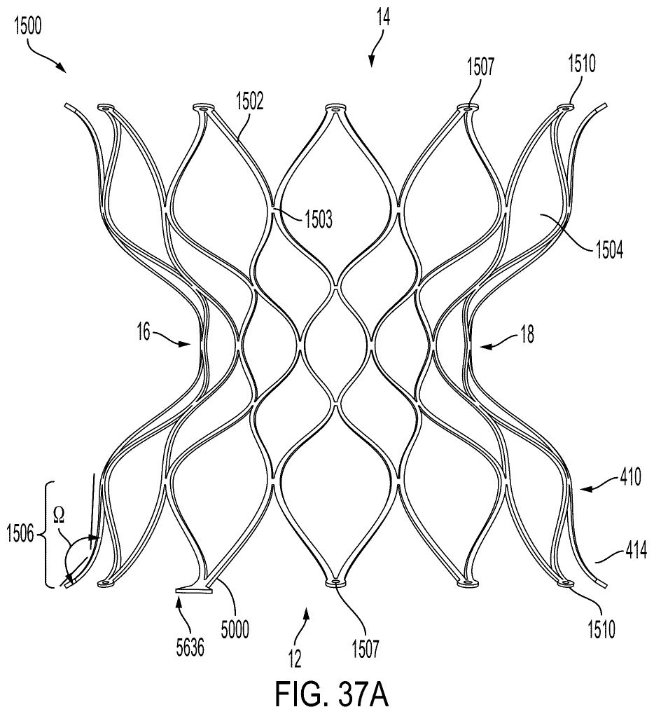

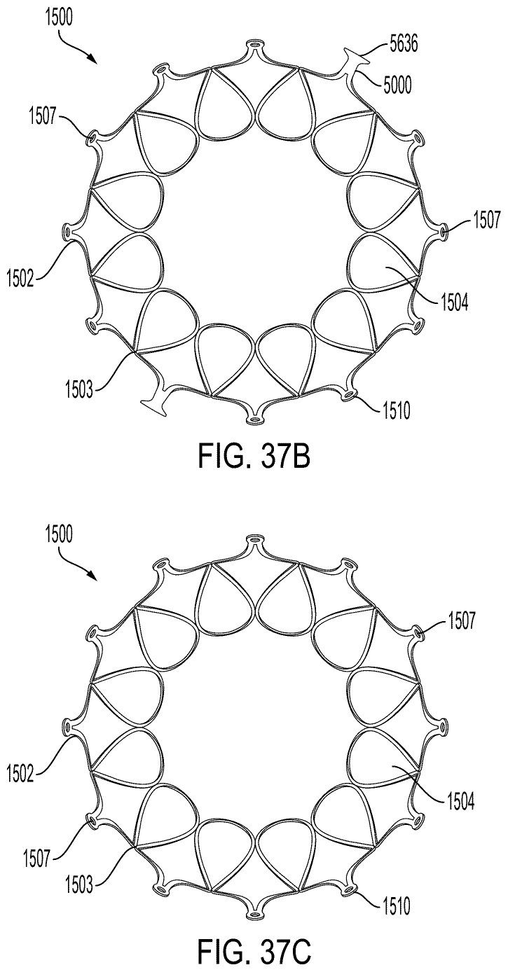

Referring to FIGS. 36A through 38B, the docking station 10 can be shaped or otherwise configured to better secure in pulmonary arteries of varying sizes, shapes, diameters, and geometries. As shown in FIGS. 36A through 37C, the frame 1500 of the docking station 10 can include different number of apices 1510 at the proximal and/or distal ends 12, 14. The number of apices 1510 can be determined to provide a better securement, fit, or apposition of the docking station 10 in the pulmonary artery PA. For example, the docking station 10 can include more apices 1510 in pulmonary arteries with larger diameters or varying geometries.

As shown in FIGS. 36A through 36C, the frame 1500 can be configured to include apices 1510 at the proximal end 12 and 14 apices 1510 at the distal end 14 which can provide better apposition in the anatomy of the pulmonary artery PA. As shown in FIGS. 37A through 37C, the frame 1500 can be configured to include apices 1510 at the proximal end 12 and 12 apices 1510 at the distal end 14 which can lower the force required to crimp the docking station 10 to fit a delivery device such as a catheter (e.g., catheter 3600 as shown in FIG. 33) or can reduce the outward radial force exerted by the docking station 10 onto the pulmonary artery PA. While the docking station 10 has been described as having either 12 or 14 apices 1510, the docking station 10 can include any number of apices 1510. For example, the docking station 10 can have 8-11 apices 1510, such as 10 apices 1510, 13 apices, 15 or more apices 1510, such as 16 apices 1510, or any other number of apices 1510. Additionally, the docking station 10 can be configured such that the proximal and distal ends 12, 14 have different numbers of apices 1510 to fit pulmonary arteries PA of varying shapes, sizes, and diameters.

The docking station 10 can also be configured to decrease or prevent additional trauma to the pulmonary artery. For example, the apices 1510 of the frame 1500 can contain a shallow angle between the sealing portions 410 and the retaining portions 414 to decrease traumatization of the tissue of the pulmonary artery while still permitting retention of the docking station 10 within the pulmonary artery PA. For example, an angle Ω at the transition between the sealing portions 410 and the retaining portions 414 can be between 120 degrees and 140 degrees, such as between 125 and 135 degrees, such as about 130 degrees. The struts 1502 which define the proximal and distal apices 1510 can be curved, bent, or otherwise shaped such that the apices 1510 are flared radially outward to a position that will maintain the docking station 10 in the pulmonary artery when the docking station 10 is deployed and will decrease or minimize trauma caused to the tissue of the pulmonary artery.

The frame 1500 of the docking station 10 can include one or more eyelets 1507 at the apices 1510. The eyelets 1507 can be circular or rounded passages or apertures extending through the frame 1500 at the proximal and/or distal ends 12, 14. As detailed below, the eyelets 1507 can be used to secure or attach the impermeable material 21 to the frame 1500. In the illustrated embodiment, the frame 1500 includes eyelets 1507 at the proximal and distal ends 12, 14. However, one or more apices 1510 at either the proximal end 12 or the distal end 14 may not have an eyelet 1507 and the apex 1510 can be generally solid and rounded. For example, the apices 1510 at the distal end 14 may not include eyelets 1507 in embodiments where the impermeable member 21 does not extend to the distal end 14, as detailed below.

The frame 1500 can also include one or more elongated legs or extensions 5000 and one or more heads 5636, as described above. The one or more elongated legs or extensions 5000 and one or more heads 5636 can facilitate the deployment, recapture, and redeployment of the docking station 10. In the illustrated embodiments, each frame 1500 includes two extensions 5000 and two heads 5636 at opposite sides of the proximal end 12. However, the frame 1500 can include extensions 5000 and heads 5636 in any number and in any suitable configuration. For example, the frame 1500 can include extensions 5000 and heads 5636 at the distal end 14 and/or the frame 1500 can have one or three or more heads 5636 at one or both of the ends 12, 14. The extensions 5000 and/or the heads 5636 can be longer than the apices 1510, while still being short enough to control the frame 1500 during deployment from the delivery device. For example, the extensions 5000 and/or the heads 5636 can be between 0.5 mm and 3.0 mm longer than the apices 1510 (or any particular length or subrange between 0.5 mm and 3.0 mm), such as between 0.8 mm and 1.8 mm (or any particular length or subrange between 0.8 mm and 1.8mm) longer than the apices 1510, such as 1.3 mm longer than the apices.

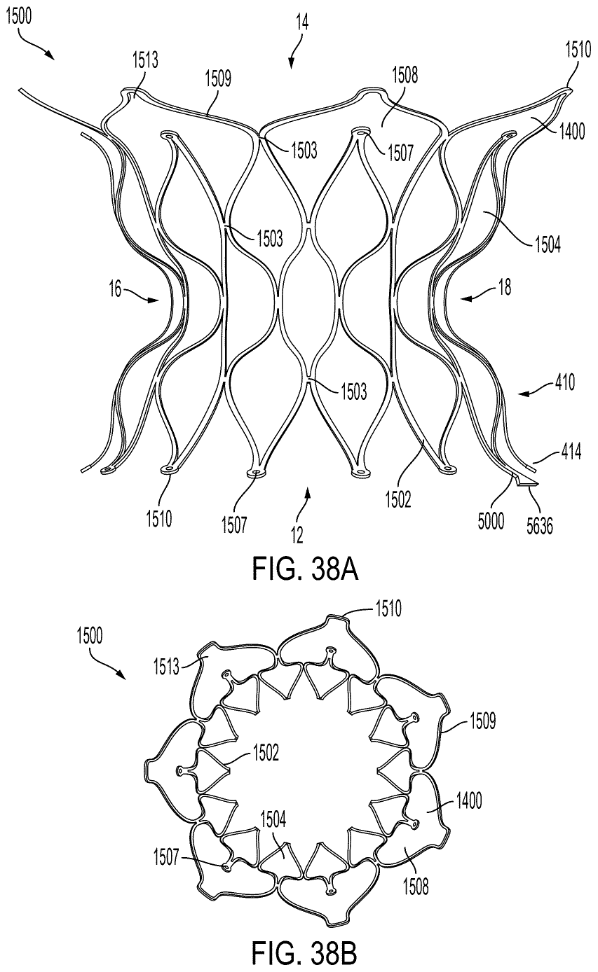

As shown in FIGS. 38A and 38B, the frame 1500 of the docking station 10 can be configured to include a plurality of outflow cells 1508 at the distal end 14 of the frame 1500 that facilitate blood flow through the docking station 10 when the docking station 10 is deployed. The outflow cells 1508 can extend into the pulmonary artery bifurcation or branch when the docking station 10 is placed higher in the pulmonary artery. At least a portion of the outflow cells 1508 may not be covered by the impermeable material 21 and the outflow cells 1508 can form at least part of the permeable portion 1400. The outflow cells 1508 can be larger than the other cells 1504 of the frame 1500. Each outflow cell 1508 can be defined by one or more outflow struts 1509. The one or more outflow struts 1509 defining the outflow cell 1508 can be shaped or otherwise configured to define one of the distal ends or apices 1510. The outflow struts 1509 of each outflow cell 1508 can extend distally from two of the distal-most junctions 1503 of the cells 1504. The outflow cells 1508 can increase the width and the stability of the frame 1500 when deployed without significantly increasing the height of the frame 1500. For example, the outflow cells can increase the height of the frame by less than 1/8^{th} of the height of the remainder of the frame, increase the height of the frame by less than 1/12^{th} of the height of the remainder of the frame, increase the height of the frame by less than 1/16^{th} of the height of the remainder of the frame, increase the height of the frame by less than 1/20^{th} of the height of the remainder of the frame, or not increase the height of the frame at all.

In the illustrated embodiments, the outflow cells 1508 are each partially defined by one outflow strut 1509 which is bent to define one of the distal ends. The ends of the outflow strut 1509 are each attached to the distal most junction 1503 between two of the distal most cells 1504 with one cell 1504 in between the two cells 1504. In such an embodiment, the frame 1500 can include eyelets 1507 at the distal most junction 1503 of the cells 1504 to which the outflow struts 1509 do not attach. In such an embodiment, each outflow cell 1508 is defined by one outflow strut 1509 and four struts 1502.

As shown in FIGS. 38A and 38B, the outflow strut 1509 can be bent, pinched, or otherwise shaped such that the distal portion of the outflow cells 1508 define a narrow end 1513. The narrow ends 1513 can help secure the deployed docking station 10 in the pulmonary artery and can be used to help crimp the docking station 10 into a delivery device such as a catheter (e.g., catheter 3600 as shown in FIG. 33).

In the illustrated embodiments, the outflow cells 1508 comprise the distal most row of cells. However, the frame 1500 can include outflow cells 1508 in any suitable configuration. For example, some but not all of the cells in the distal most row can be outflow cells 1508 or the outflow cells 1508 can constitute two or more rows of cells.

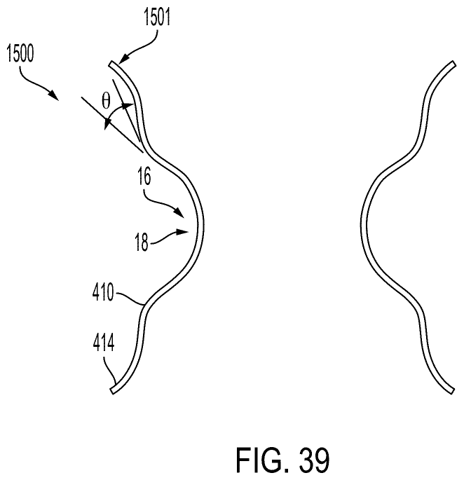

Referring now to FIG. 39, any of the frames 1500 described herein can be configured such that the frame 1500 is easier to deploy, recapture, and/or redeploy. For example, the frame 1500 can be configured to reduce the amount of force required to recapture frame 1500. As shown in FIG. 39, any of the frames 1500 described herein can have a side profile with a maximum transition angle Θ at any location along the frame 1500. The maximum transition angle Θ defines the maximum angle between tangent lines at close points along the frame 1500. For example, the maximum transition angle can be measured as the angle between tangents of any two points that are 0.1 mm apart along the profile of the frame. The frame 1500 can be shaped and configured and the maximum transition angle Θ set such that the docking station 10 can be easily deployed, recaptured, and redeployed. The frame 1500 can be configured such that the maximum transition angle Θ is minimized and large internal forces within the frame 1500 required to compress the frame back into the catheter do not prevent the frame 1500 from being recaptured or redeployed. For example, the side profile 1501 is shaped and configured such that the maximum transition angle Θ is less than 60°, such as less than 55°, such as less than 50°, such as 45°.

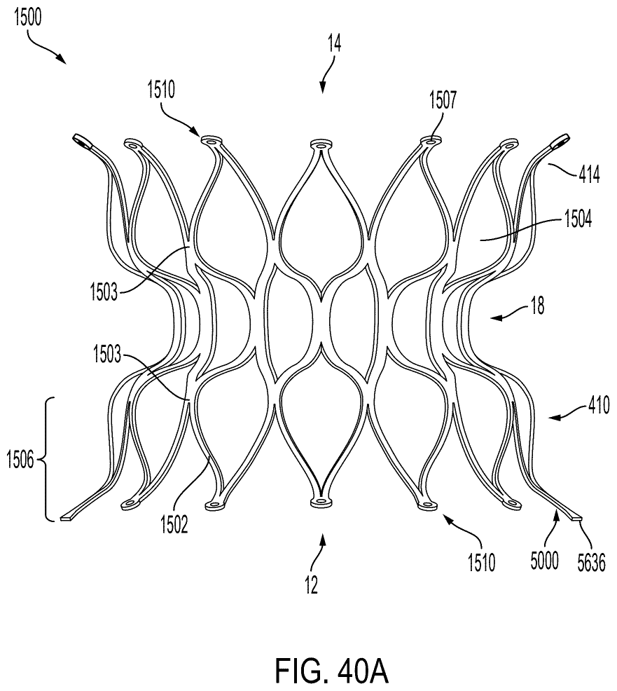

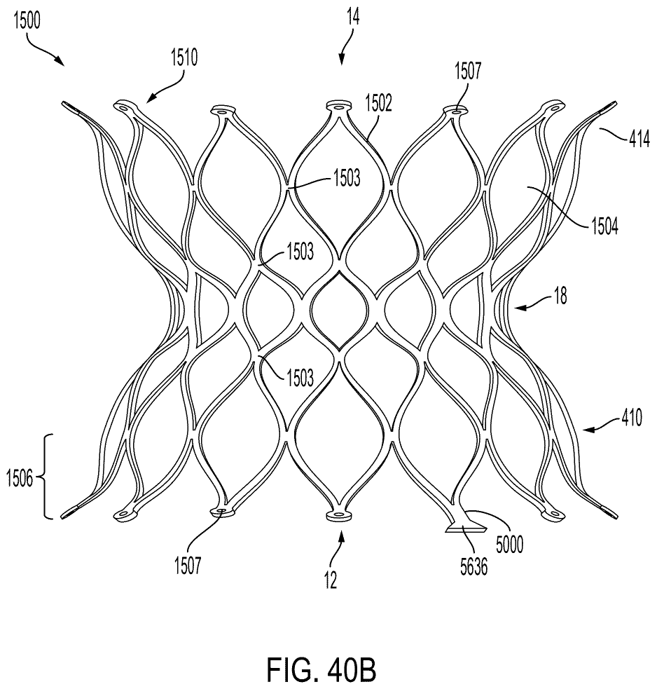

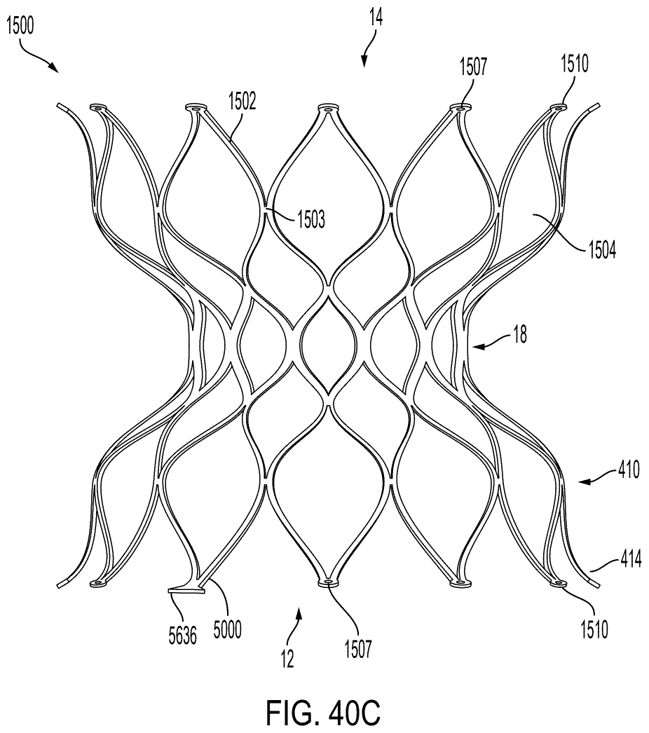

Referring now to FIGS. 40A, 40B, and 40C, the struts 1502 of the docking station 10 can be configured to provide a more resilient valve seat 18 or to provide more radial force against the valve 29 when the valve 29 is deployed in the valve seat 18. In some exemplary embodiments, the frame 1500 can be configured such that the band 20 (see FIG. 11) can be omitted. Additionally or alternatively, the frame can be configured such that the impermeable member 21 does not include additional stitching which can increase the radial resistance as described below. As shown in FIGS. 40A-40C, the struts 1502 in the rungs 1506 near the valve seat 18 can be thicker or have an increased cross-sectional width or diameter in relation to the struts 1502 of other portions of the frame 1500. In the illustrated embodiments, the struts 1502 of the two rungs 1506 in the middle of the frame 1500 (i.e. in the area of the valve seat 18) are thicker than the struts 1502 of the other rungs 1506. However, the frame 1500 of the docking station 10 can have a variety of other configurations to provide a more resilient valve seat 18 or to provide more radial force against the valve 29 when the valve 29 deployed in the valve seat 18. For example, the struts 1502 of any other rung 1506 can also have an increased cross-sectional width or diameter or not all of the struts 1502 of the middle two rungs 1506 can have an increased cross-sectional width or diameter.

While the docking station 10 has been described as having thicker struts 1502 or struts 1502 with an increased cross-sectional width or diameter to provide a more resilient valve seat 18 and/or to assert more radial force against the deployed valve 29, the docking station 10 can be configured in other ways to provide the same effect. For example, the portions of the struts 1502 and/or frame 1500 near the valve seat 18 can comprise a stronger, less elastic, and/or more resilient metal or material, the junctions 1503 near the valve seat 18 can be stronger and/or thicker, or the lattice structure of the frame 1500 can be stronger near the valve seat 18, such as by increasing the number and decreasing the length of the struts 1502 in the rungs 1506 near the valve seat 18.

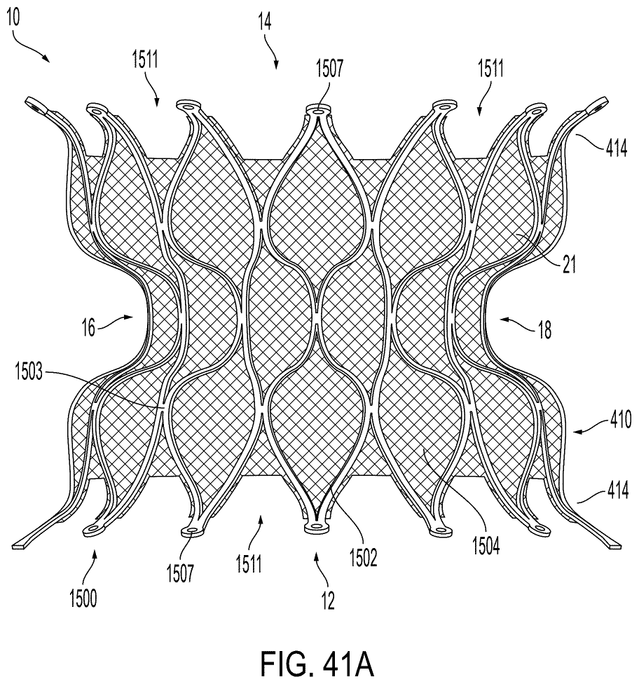

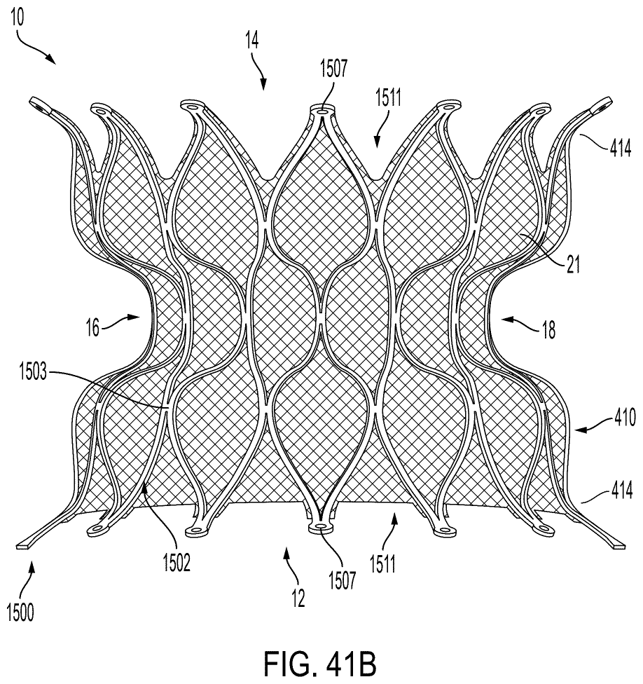

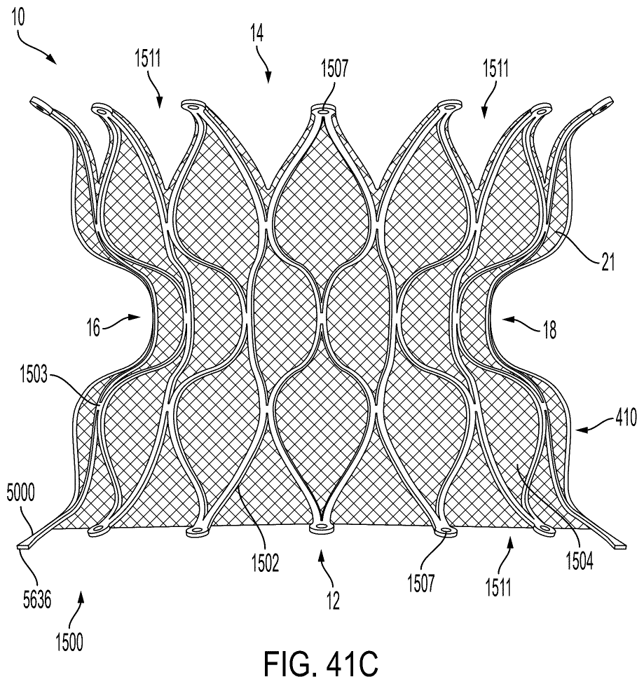

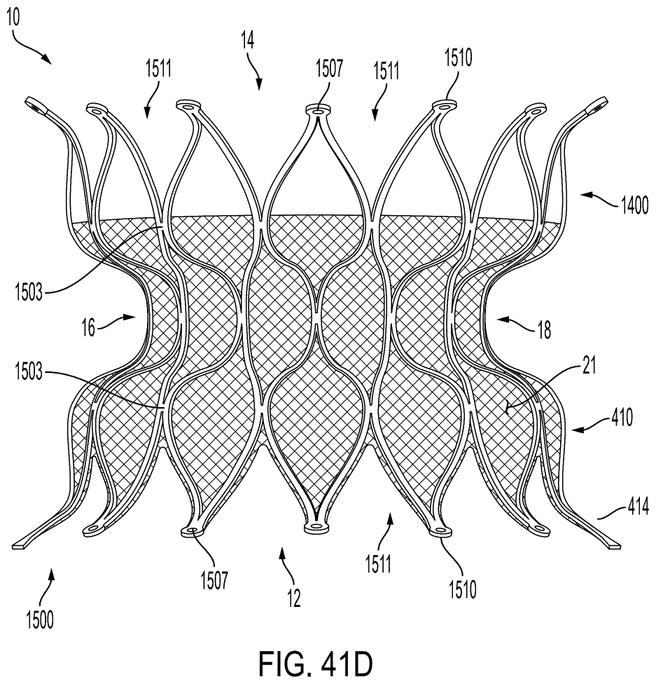

Referring now to FIGS. 41A through 41D, the cloth or impermeable material 21 can be cut, configured, or otherwise shaped such that the impermeable material 21 does not bunch and/or tear when the docking station 10 is compressed or deployed. The impermeable material 21 can be cut, configured, or otherwise shaped such that the impermeable material 21 does not cover at least a portion of the frame 1500 near the proximal end 12 and/or the distal end 14. The impermeable material 21 can be cut or shaped such that the impermeable material 21 does not cover at least a portion of the space not defined by one of the cells 1504 near the proximal and/or distal ends 12, 14. The impermeable material 21 can be configured or cut to the desired shape before the impermeable material 21 is attached to the frame 1500 or the impermeable material 21 can be attached to the frame 1500 and then cut to the desired shape.

At the proximal and distal ends 12, 14, the frame 1500 can include a plurality of openings 1511 between the struts 1502 and the apices 1510 in the portions of the frame 1500 which are not defined by the cells 1504. The openings 1511 are generally triangular in shape and are partially defined by two struts 1502, two apices 1510, and a junction 1503. The impermeable material 21 can be cut or shaped such that the impermeable material 21 does not cover at least a portion of the openings 1511 at the proximal and/or distal ends 12, 14.

The impermeable material 21 can be cut, configured, or otherwise shaped in a wide variety of ways such that the impermeable material 21 does not bunch or tear when the docking station 10 is compressed or deployed. The impermeable material 21 can be cut or shaped such that the impermeable material 21 can be attached to or disposed on the frame 1500 such that the impermeable material 21 can cover at least a portion of the cells 1504 but not cover at least a portion of the openings 1511 at the proximal and/or distal ends 12, 14.

As shown in FIG. 41A, the impermeable material 21 can be shaped or cut such that the impermeable material 21 substantially covers each cell 1504, substantially covers one-half of each opening 1511 at the proximal end 12, and substantially covers one-half of each opening 1511 at the distal end 14. However, the impermeable material 21 can be shaped or cut such that the impermeable material 21 substantially covers each cell 1504, substantially covers one-fourth, one-third, two-third, three-fourths, or any other suitable amount of each opening 1511 at the proximal end 12, and substantially covers one-fourth, one-third, two-third, three-fourths, or any other suitable amount of each opening 1511 at the distal end 14. Referring back to FIGS. 16A and 16B, the docking stations 10 can be used in differently sized circulatory system anatomies. By removing a portion of the material 21 in the opening 1511 at the proximal and/or distal end, the material 21 in the opening will not bunch up or the bunching up will be reduced when the docking station is used in a smaller circulatory system anatomy (e.g. FIG. 16B).

As shown in FIG. 41B, the impermeable material 21 substantially covers each cell 1504, substantially covers three-fourths of each opening 1511, at the proximal end 12 and generally does not cover the openings 1511 at the distal end 14.

As shown in FIG. 41C, the impermeable material 21 substantially covers each cell 1504, substantially covers the openings 1511 at the proximal end 12, and generally does not cover the openings 1511 at the distal end 14.

In each of the illustrated embodiments, the impermeable material 21 is cut horizontally or straight across. However, the impermeable material 21 can be cut or shaped in any suitable direction or pattern. For example, the impermeable material 21 can be cut or shaped in a rounded or sinusoidal pattern. Additionally, the impermeable material 21 has been described as covering each of the openings 1511 at the proximal end 12 in a uniform manner and covering each of the openings 1511 at the distal end 14 in a uniform manner. However, the impermeable material 21 can be cut or shaped such that the openings 1511 at each end 12, 14 are not covered in a uniform manner. For example, each of the openings 1511 at either end 12, 14 can be covered in a different manner or amount than the other openings 1511. Further, the impermeable material 21 can be cut or shaped larger than desired such that the impermeable material 21 can be disposed on or affixed to the struts 1502, as detailed below.

The impermeable material 21 can also be cut or otherwise shaped such that the impermeable material 21 does not cover at least a portion of the distal most cells 1504 or the outflow cells 1508. In such an embodiment, a portion of the distal most cells 1504 or the outflow cells 1508 and the openings 1511 can form the permeable portion 1400. As shown in FIG. 41D, the impermeable material 21 can be cut or shaped such that the impermeable material 21 substantially covers the proximal most cells 1504, generally does not cover the openings 1511 at the proximal end 12, substantially covers one-half of each of the distal most cells 1504, and generally does not cover the openings 1511 at the distal end 14. The impermeable material 21 can be cut or otherwise shaped such that the impermeable material 21 extends horizontally across at a point substantially equivalent to the location of the distal most junctions 1503.

In the embodiment illustrated by FIG. 41D, the impermeable cover 21 substantially covers one-half of the distal most cells 1504. However, the impermeable cover 21 can cover any amount of the distal most cells 1504. For example, the impermeable cover 21 can be cut or shaped to cover one-fourth, one-third, two-third, three-fourths, or any other suitable amount of the distal most cells 1504. In the illustrated embodiment, the impermeable material 21 generally does not cover the openings 1511 at the proximal end 12. However, the impermeable material 21 can cover the openings 1511 at the proximal end 12 in any amount or manner, such as the ways depicted and described in FIGS. 41A, 41B, and 41C. Additionally, while the impermeable material 21 is depicted as extending horizontally across the distal most junctions, the impermeable material 21 can have any other suitable shape extending across the distal most cells 1504 and junctions 1503. For example, the impermeable material 21 can have a rounded, curved, sinusoidal, or any other cut or shape extending across the distal most cells 1504 and junctions 1503.

While the various configurations of the impermeable material 21 have been described and illustrated as being used with the four rung 1506 frame 1500 of FIG. 34, the various configurations of the impermeable material 21 can be applied with any other docking station 10 described herein. For example, the various configurations of the impermeable material 21 can be used with the six rung 1506 frame 1500 of FIGS. 35-37C, with the frame 1500 having outflow cells 1508 of FIGS. 38A and 38B, the frame 1500 with thicker struts 1502 of FIGS. 40A-40C, or any other frame 1500 described herein.

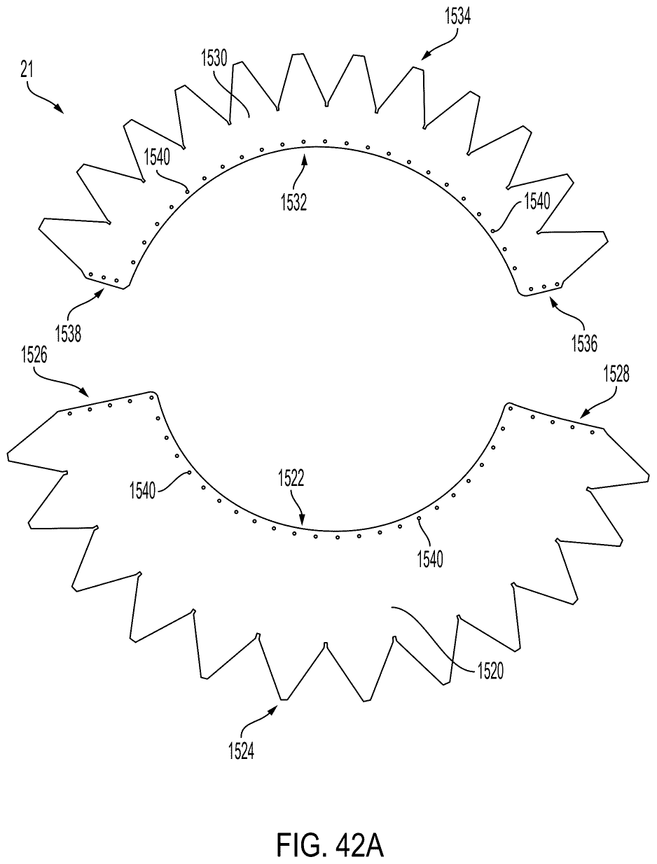

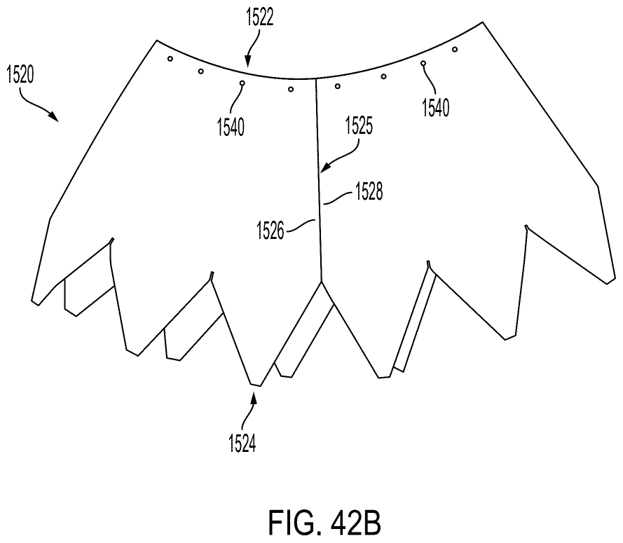

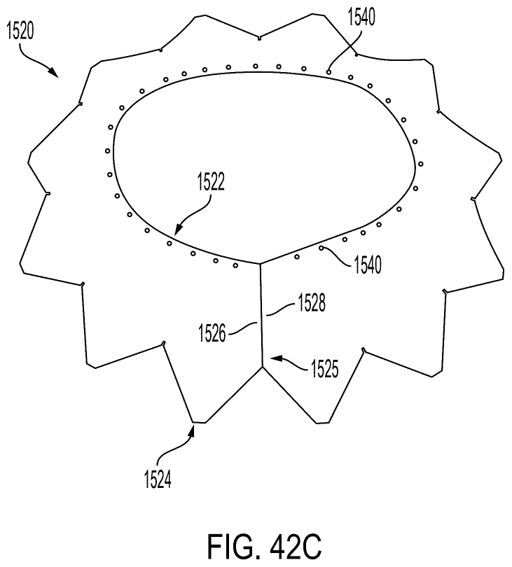

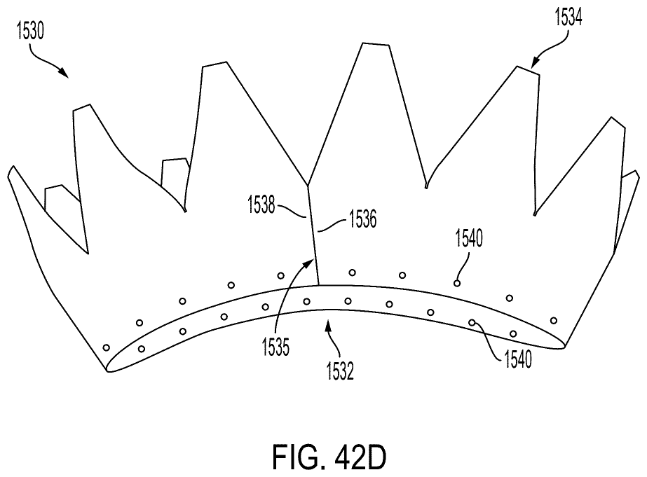

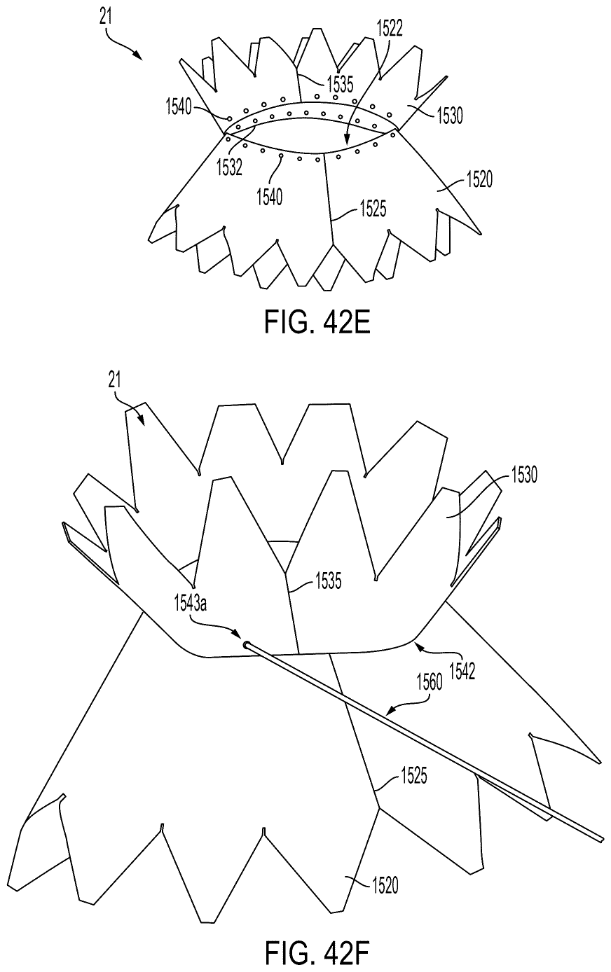

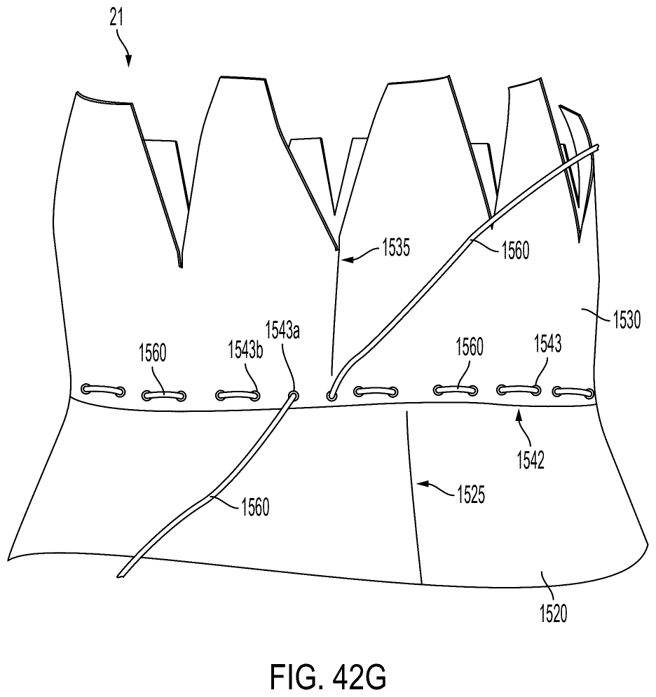

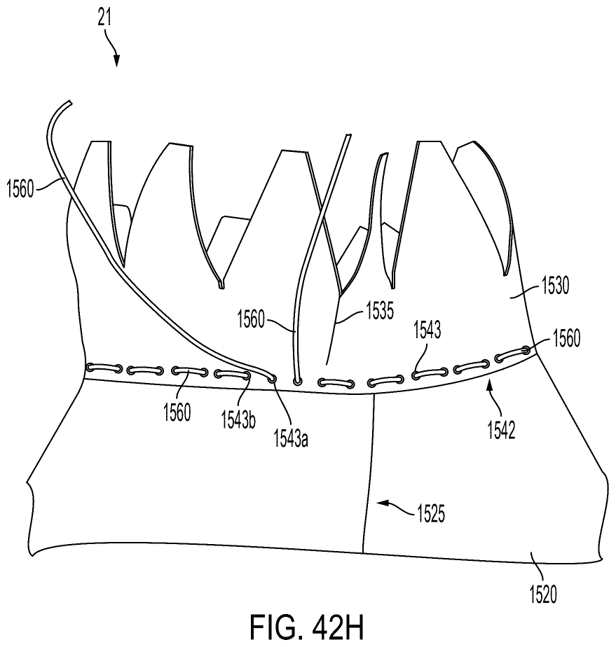

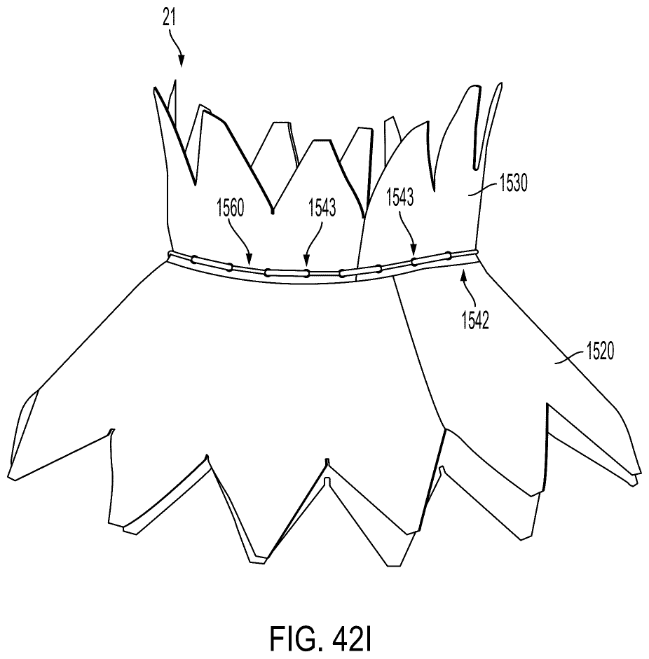

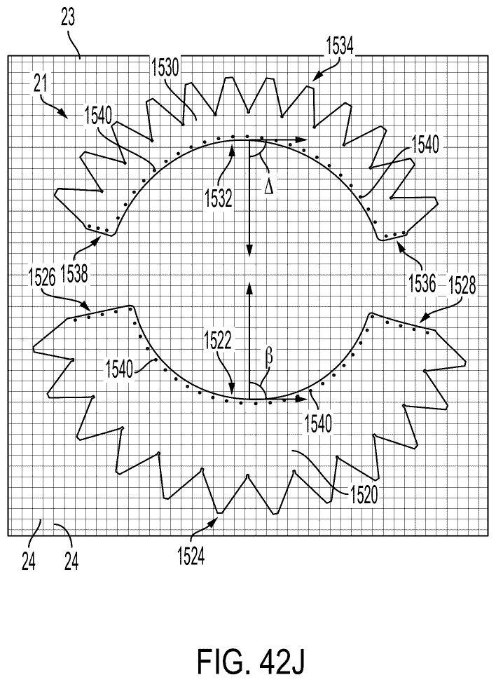

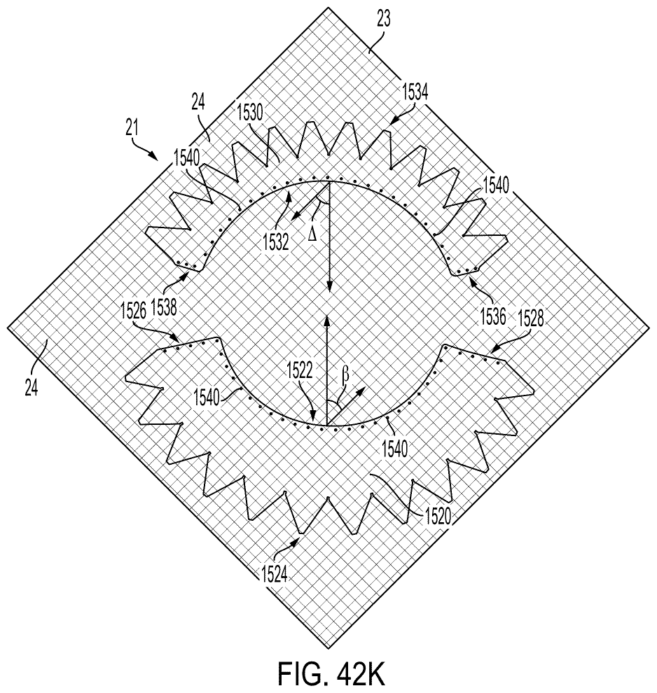

Referring now to FIGS. 42A to 44I, the impermeable material 21 can be attached to, secured around, or otherwise affixed to the frame 1500 of the docking station 10 in a variety of ways. For example, the impermeable material can be affixed to the frame 1500 using sewing or electrospinning or the impermeable material 21 can be made from a suture-less material.

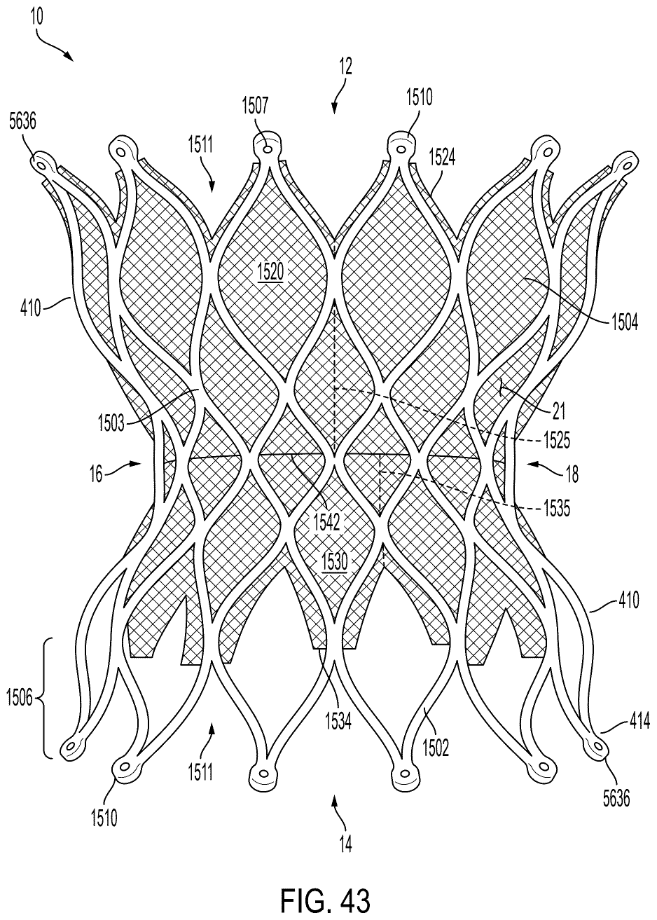

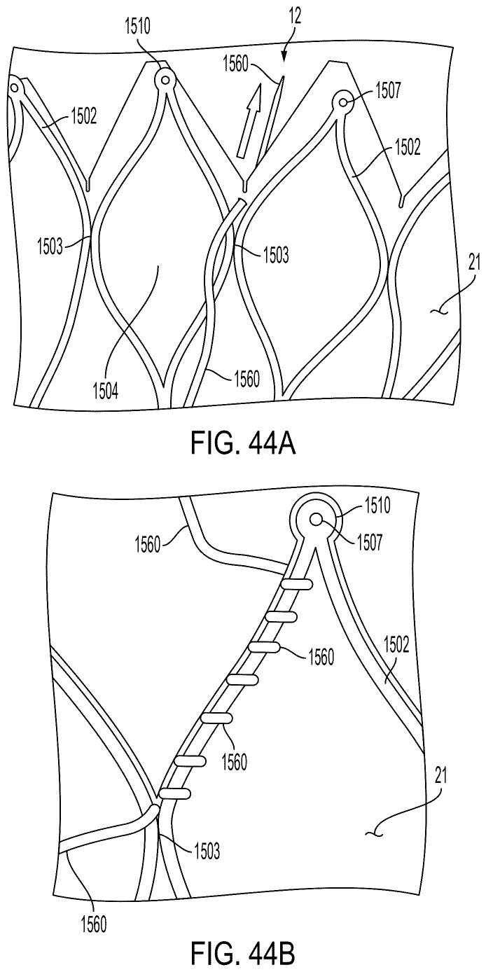

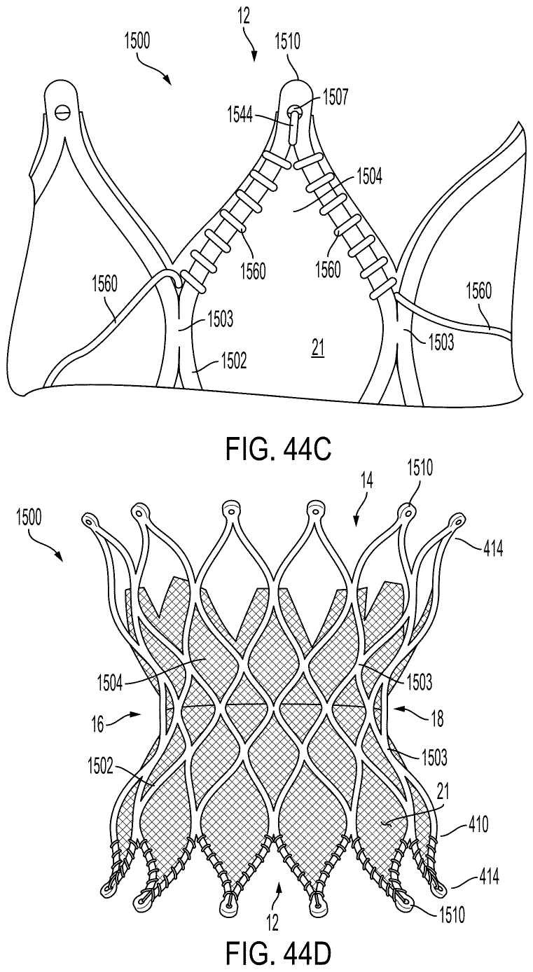

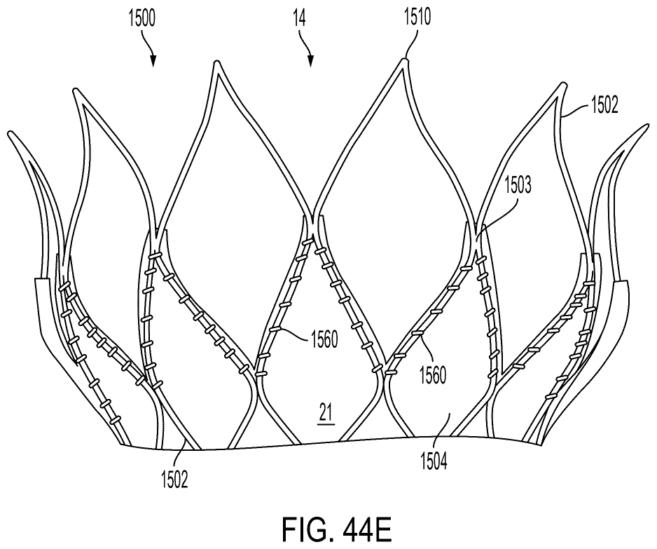

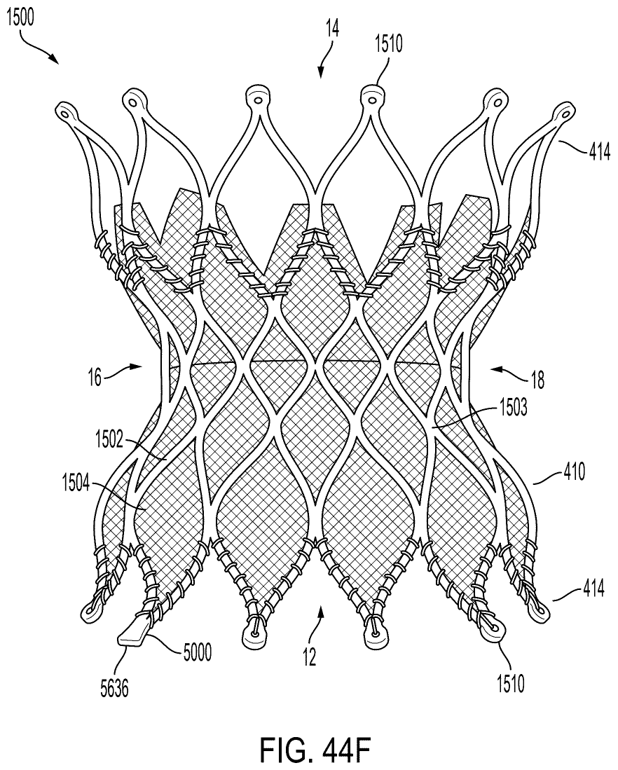

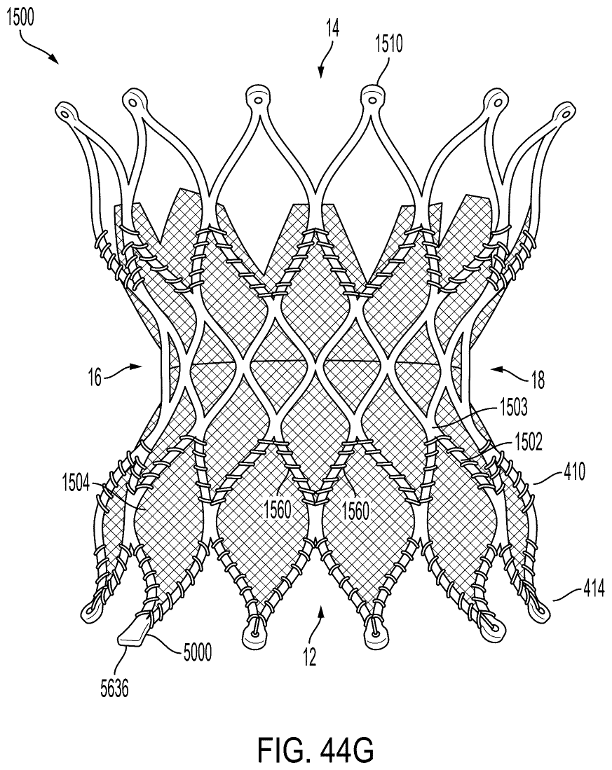

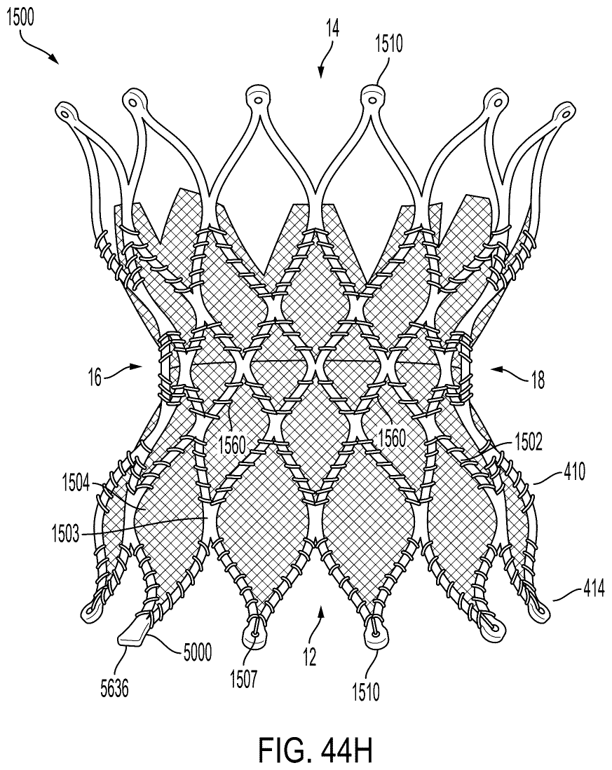

As shown in FIGS. 42A through 44I, the impermeable material 21 can be affixed to the frame 1500 by sewing one or more pieces of impermeable material 21 together and then onto the frame 1500. As shown in FIGS. 43 through 44I, the frame 1500 can include one or more eyelets 1507 at the apices 1510 which can facilitate attaching the impermeable material 21 to the frame 1500. In the illustrated embodiment, each apex 1510 that does not include an elongated leg 5000 includes an eyelet 1507. However, the number of eyelets 1507 can vary and each apex 1510 may not include either an elongated leg 5000 or an eyelet 1507. For example, the apices 1510 at the distal end 14 may not have any eyelets 1507 or elongated legs 5000.

As shown in FIGS. 42A through 42I, the impermeable cover 21 can have a proximal portion 1520 and a distal portion 1530. The proximal portion 1520 can be sized and shaped to cover the desired portion of the frame 1500 between the valve seat 18 and the proximal end 12. The distal portion 1530 can be sized and shaped to cover the desired portion of the frame 1500 between the valve seat 18 and the distal end 14. In the illustrated embodiment, the impermeable member 21 has two portions 1520, 1530. However, the impermeable member 21 can have any number of portions which are secured together form the impermeable member 21. For example, the impermeable member can be made from a single piece or have three, four, five, or more portions.

The proximal portion 1520 has a first edge 1522, a second edge 1524, a first end 1526, and a second end 1528 and the distal portion 1530 has a first edge 1532, a second edge 1534, a first end 1536, and a second end 1538. As detailed below, the first edges 1522, 1532 can be sized and shaped to fit the valve seat 18 of the frame 1500, the second edge 1524 of the proximal portion 1520 can be sized and shaped to fit the frame 1500 at the desired position between the valve seat 18 and the proximal end 12, and the second edge 1534 of the distal portion 1530 can be sized and shaped to fit the frame 1500 at the desired position between the valve seat 18 and the distal end 14. In the illustrated embodiment, the proximal and distal portions 1520, 1530 are shaped such that first ends 1526, 1536 are generally in the shape of the apices 1510. However, the proximal and distal portions 1520, 1530 can be shaped in a wide variety of ways. For example, the proximal and distal portions 1520, 1530 can be shaped or otherwise configured such that the impermeable material 21 has any of the shapes or configurations illustrated and described in FIGS. 41A-41D.

As shown in FIGS. 42B and 42C, the first end 1526 of the proximal portion 1520 can be folded or looped around and secured to the second end 1528 of the proximal portion 1520 to form a proximal portion joint 1525.

As shown in FIG. 42D, the first end 1536 of the distal portion 1530 can be folded or looped around and secured to the second end 1538 of the distal portion 1530 to form a distal portion joint 1535. The first ends 1526, 1536 can be secured to the second ends 1528, 1538 in any suitable manner. For example, the first ends 1526, 1536 can be secured to the second ends 1528, 1538 by sewing a thread or suture, by an adhesive, by a fastener, or by any other suitable means.

As shown in FIGS. 42E through 42I, the proximal portion 1520 can be secured to the distal portion 1530 such that the second edge 1524 of the proximal portion 1520 is opposite the second edge 1534 of the distal portion 1530. The first edge 1522 of the proximal portion 1520 can overlap the first edge 1532 of the distal portion 1530 and can create a medial joint 1542. In one embodiment, the proximal portion joint 1525 is not aligned with the distal portion joint 1535 when the proximal and distal portions 1520, 1530 are secured. Offsetting the proximal and distal portion joints 1525, 1535 can increase the ease of manufacture of the impermeable member 21 and/or increase the strength and resilience of the impermeable member 21. The proximal portion joint 1525 can be offset from the distal portion joint 1535 such that the proximal portion joint 1525 aligns with one of the apices 1510 and/or junctions 1503 and the distal portion joint 1535 aligns with another one of the apices 1510 and/or junctions 1503. For example, the proximal portion joint 1525 and distal portion joints 1535 can be offset so that the proximal and distal portion joints 1525, 1535 can each run along one of the junctions 1503 when the impermeable member 21 is attached to the frame 1500.

The proximal portion 1520 can be secured to the distal portion 1530 in any suitable manner. For example, the first edge 1522 of the proximal portion 1520 can be secured to the first edge 1532 of the distal portion 1530 by sewing a thread or suture, by an adhesive, by a fastener, or by any other suitable means.

In one embodiment, the proximal and distal portions 1520, 1530 each include a plurality of apertures 1540 along the first edge 1522, 1532, the first end 1526, 1536, and the second end 1528, 1538. The apertures 1540 may facilitate the assembly of the impermeable material 21, such as by serving as guides for a suture or thread to be sewn therethrough. The apertures 1540 can be formed by any suitable process, such as cutting or laser drilling.

As shown in FIGS. 42E through 42I, the proximal portion 1520 can be attached to the distal portion 1530 near the medial joint 1542 by an interlocking stitch which provides radial force against the valve 29 when the valve 29 is deployed in the valve seat 18. The proximal portion 1520 can be positioned in line with or on top of the distal portion 1530 such that the first edges 1522, 1532 overlap. A suture 1560 can be passed through (radially inwardly) the proximal and distal portions 1520, 1530 between the first edges 1522, 1532 at a first point 1543a. The suture 1560 can then be passed back through (radially outwardly) the proximal and distal portions 1520, 1530 in the opposite direction at a second point 1543b circumferentially spaced apart from the first point 1543a. The suture 1560 can then be repeatedly passed in and out through the proximal and distal portions 1520, 1530 at other points 1543 until the suture 1560 substantially spans the circumference of the proximal and distal portions 1520, 1530.

Once the suture 1560 has been passed substantially around the circumference of the proximal and distal portions 1520, 1530, the suture 1560 can be passed back through the proximal and distal portions 1520, 1530 in the opposite direction. When the suture 1560 is passed back through the proximal and distal portions 1520, 1530 in the opposite direction, the suture 1560 can be passed through the proximal and distal portions 1520, 1530 at the same points 1543 such that the suture 1560 fills the spaces between the previous stitches of the suture 1560 along or near the medial joint 1542. As such, a circumferential portion of the impermeable material 21 at or near the medial joint 1542 can be substantially covered by the suture 1560 on both sides of the impermeable material 21.

As shown in FIGS. 42J and 42K, the proximal portion 1520 and the distal portion 1530 can be cut, shaped, or otherwise formed from one or more pieces of cloth 23. The cloth 23 can include fibers 24 generally disposed vertically and horizontally when the cloth 23 is oriented vertically. In the illustrated embodiment, the proximal portion 1520 and the distal portion 1530 are cut from the same cloth 23. However, the proximal portion 1520 can be cut from a first cloth 23 and the distal portion 1530 can be cut from a second cloth 23.

The proximal portion 1520 can be cut from the cloth 23 such that an angle β is formed between the horizontally oriented fibers 24 and a line normal to the center of the first edge 1522 of the proximal portion 1520. Alternatively, a cloth can be selected that has fibers that are oriented with the angle β. The proximal portion 1520 can be cut (or fiber orientation can be selected) in a manner that increases the strength and resiliency of the proximal portion 1520 and/or facilitates the assembly of the impermeable member 21 and the attachment of the impermeable member 21 to the frame 1500. In one embodiment, as shown in FIG. 42J, the proximal portion 1520 can be cut such that the angle β is approximately 90°. In another embodiment, as shown in FIG. 42K, the proximal portion 1520 can be cut such that the angle β is between 20° and 70°, such as between 30° and 50°, such as 45°.

The distal portion 1530 can be cut from the cloth 23 such that an angle Δ is formed between the horizontally oriented fibers 24 and a line normal to the center of the first edge 1532 of the distal portion 1530. Alternatively, a cloth can be selected that has fibers that are oriented with the angle Δ. The distal portion 1530 can be cut (or fiber orientation can be selected) in a manner that increases the strength and resiliency of the distal portion 1530 and/or facilitates the assembly of the impermeable member 21 and the attachment of the impermeable member 21 to the frame 1500. In one embodiment, as shown in FIG. 42J, the distal portion 1530 can be cut such that the angle Δ is approximately 90°. In another embodiment, as shown in FIG. 42K, the proximal portion 1520 can be cut such that the angle Δ is between 90° and 20° and 70°, such as between 30° and 50°, such as 45°.

Forming the proximal and distal portions 1520, 1530 with the fibers 24 of the cloth 23 at an angle can improve the strength or resiliency of the impermeable member 21 and/or can facilitate the assembly of the impermeable member 21. In the illustrated embodiments, the angle β and the angle Δ are substantially the same. However, the angle β and the angle Δ can be substantially different.

As shown in FIG. 43, the impermeable material 21 can be properly positioned or disposed within the frame 1500. The impermeable material 21 can be positioned such that the medial joint 1542 is substantially aligned in the middle of the valve seat 18 of the frame 1500. The impermeable material 21 can also be positioned such that the second edge 1524 of the proximal portion 1520 is substantially aligned with the desired struts 1502, junctions 1503, and/or apices 1510 near the proximal end 12 and the second edge 1534 of the distal portion 1530 is substantially aligned with the desired struts 1502, junctions 1503, and/or apices 1510 near the distal end 14. In the illustrated embodiment, the impermeable material 21 extends from the proximal end 12 of the frame 1500 toward the distal end 14 and does not extend to the distal most row of cells 1504. The impermeable material 21 can also be configured such that the impermeable material 21 does not cover the openings 1511 at the proximal end 12. However, the impermeable material 21 can be sized and shaped in any suitable configuration. For example, the impermeable material 21 can extend to the distal end 14 of the frame 1500 and the impermeable material 21 can cover the cells 1504 and openings 1511 near the ends 12, 14 in any amount or configuration, such as the configurations depicted and described in FIGS. 41A-41D.

Optionally, the impermeable member 21 can be configured and/or positioned such that the proximal portion joint 1525 and distal portion joint 1535 increase the strength or resiliency of the docking station 10 or facilitate the attachment of the impermeable member 21 to the frame 1500. As shown by the dotted lines in FIG. 43, the impermeable member 21 can be configured and/or positioned such that the proximal portion joint 1525 is aligned with one of the apices 1510 and/or one or more junction 1503. The impermeable member 21 can also be configured and/or positioned such that the distal portion joint 1535 is aligned with one of the apices 1510 and/or one or more junction 1503. The proximal portion joint 1525 can be aligned with different apices 1510 and/or junctions 1503 than the distal portion joint 1535. For example, the proximal portion joint 1525 can be offset from the distal portion joint 1535 by one junction 1503. In the illustrated embodiment, the proximal and distal portion joints 1525, 1535 are aligned with junctions 1503 and not with any apices 1510. However, the proximal and distal portion joints 1525, 1535 can be arranged and/or configured in a variety of ways. For example, one of or both of the proximal and distal portion joints 1525, 1535 can be respectively aligned with one of the apices 1510.

Referring now to FIGS. 44A through 44I, the impermeable material 21 can be affixed to the frame 1500 by one or more threads or sutures 1560. As shown in FIGS. 44A through 44D, the impermeable material 21 can be affixed to the proximal end 12 of the frame 1500. The impermeable material 21 can be positioned such that the proximal portion of the impermeable material 21 aligns with the desired proximal rung 1506, apices 1510, or junctions 1503. The one or more threads or sutures 1560 can be sewn or looped around the struts 1502 of the proximal most rung 1506. At a point near the proximal most junction 1503 to which the impermeable material 21 extends, the suture 1560 can be passed through the impermeable material 21, looped around the strut 1502, and passed back through the impermeable material 21 on the other side of the strut 1502. This stitch can be repeated until the suture 1560 substantially extends the length of the strut 1502. Near the apex 1510, the stitch can be repeated such that the suture 1560 descends down the subsequent strut 1502 in the rung until the suture 1560 substantially extends to the junction 1503. This stitch can be repeated until the suture 1560 substantially extends along each strut 1502 in the proximal most rung 1506 and the suture 1560 substantially extends circumferentially around the frame 1500.

As shown in FIGS. 44E and 44F, the impermeable material 21 can be affixed near the distal end 14 of the frame 1500. The impermeable material 21 can be positioned such that the distal portion of the impermeable material 21 aligns with the desired distal rung 1506, apices 1510, or junctions 1503. In the illustrated embodiment, at a point near the junction 1503 that defines the proximal end of the distal most cell 1504, the suture 1560 can be passed through the impermeable material 21, looped around the strut 1502, and passed back through the impermeable material 21 on the other side of the strut 1502. This stitch can be repeated until the suture 1560 substantially extends the length of the strut 1502. Near the distal most junction 1503 to which the impermeable material 21 extends, the stitch can be repeated such that the suture 1560 descends down the subsequent strut 1502 in the rung 1506 until the suture 1560 substantially extends to the junction 1503. This stitch can be repeated until the suture 1560 substantially extends along each strut 1502 in the distal most rung 1506 to which the impermeable material 21 extends and the suture 1560 substantially extends circumferentially around the frame 1500.

As shown in FIGS. 44G through 44I, similar stitches to the stitches described in FIGS. 44A through 44F can be used to secure the impermeable material 21 to the remaining rungs 1506 by one or more sutures 1560. The stitches can be at any angle in relation to the struts 1502 of the frame. For example, the stitches can form an angle with the struts 1502 between 45° and 90°. In the illustrated embodiment, one or more sutures 1560 secures the impermeable material 21 to each strut 1502 of each rung 1506 which the impermeable material 21 covers. However, the impermeable material 21 may not be secured to each strut 1502 of each rung 1506 which the impermeable material 21 covers. For example, the impermeable material 21 may not be secured or attached to each strut 1502 in each covered rung 1506 and/or the impermeable material 21 may not be secured or attached to some of the rungs 1506.

As shown in FIG. 44C, the impermeable material 21 can be additionally secured to the frame 1500 with one or more vertical stitches 1544. After the suture 1560 has been stitched around one of the struts 1502 in the proximal most rung 1506 and the suture 1560 substantially extends from the junction 1503 to the apex 1510, the suture 1560 can be passed through the impermeable material 21, through the eyelet 1507, and back through the impermeable material 21 to form the vertical stitch 1544. The suture 1560 can then be stitched around the descending strut 1502 toward the junction 1503. While the vertical stitch 1544 has only been depicted as securing the impermeable material 21 to the eyelets 1507 at the apices 1510 at the proximal end 12, the vertical stitch 1544 can be used at other locations of the frame 1500. For example, vertical stitches 1544 can be used in embodiments where the impermeable material 21 extends to the apices 1510 at the distal end 14 or in embodiments where the frame 1500 includes outflow cells 1508 and the impermeable material 21 extends to apices 1510 near the distal end 14. However, the impermeable material 21 may not be secured to the frame 1500 with one or more vertical stitches (e.g., FIG. 44I).

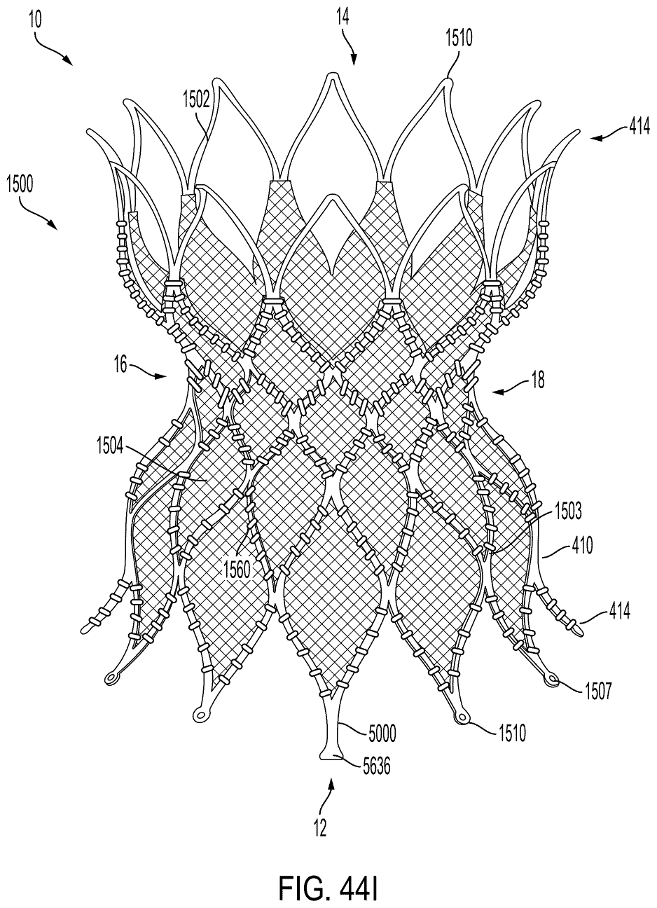

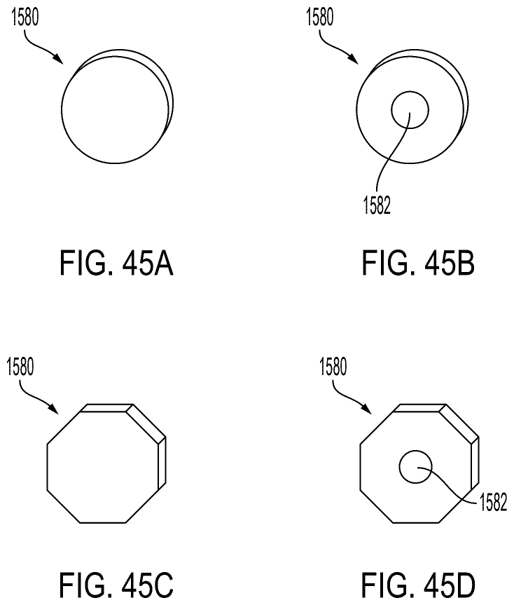

Referring now to FIGS. 45A through 45D, the docking station 10 can include one or more radiopaque markers 1580 which can assist with deployment of the docking station 10 as well as placement of the valve 29 into the valve seat 18. The one or more radiopaque markers 1580 can be radiopaque or have a higher radiopacity such that the one or more radiopaque markers 1580 can be identified under fluoroscopy or a similar imaging process. The one or more radiopaque markers 1580 can be disposed on, attached to, or otherwise affixed to the docking station 10 in a wide variety of ways, such as the ways detailed below. The one or more radiopaque markers 1580 can comprise any material or combination of materials that are radiopaque or increase the radiopacity of at least a portion of the valve seat 18. For example, the one or more radiopaque markers 1580 can comprise barium sulfate, bismuth, tungsten, tantalum, platinum-iridium, gold or any other material which is opaque to fluoroscopy, X-rays, or similar radiation or any combination thereof. As illustrated in FIGS. 45A-45D, the radiopaque markers 1580 are disc-shaped and circular or octagonal. However, the one or more radiopaque markers 1580 can be configured to reduce axial motion and can be any suitable shape. For example, the one or more radiopaque markers 1580 can be hexagonal, triangular, rectangular, elliptical, 3D, or any other shape or configuration. The radiopaque markers 1580 can also include an aperture 1582 extending through a central portion of the marker 1580. The aperture 1582 can be sized such that a suture can pass therethrough.

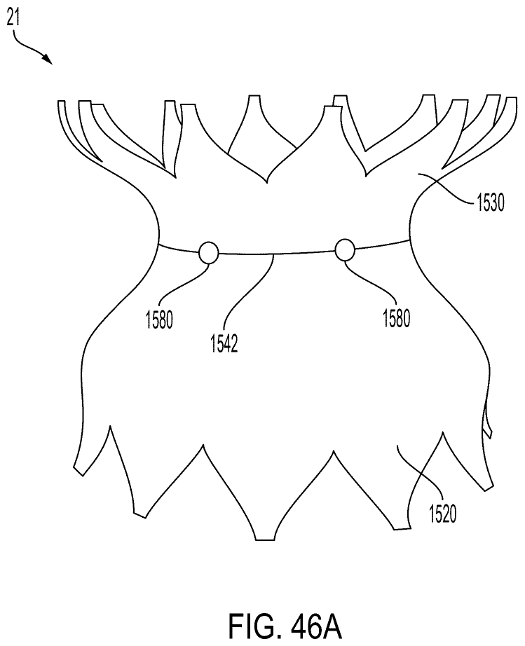

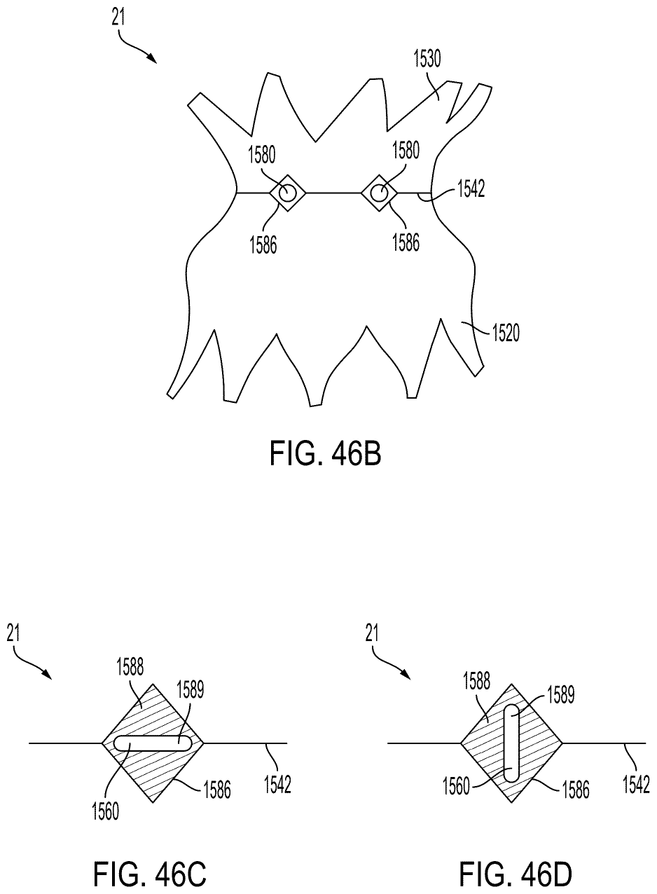

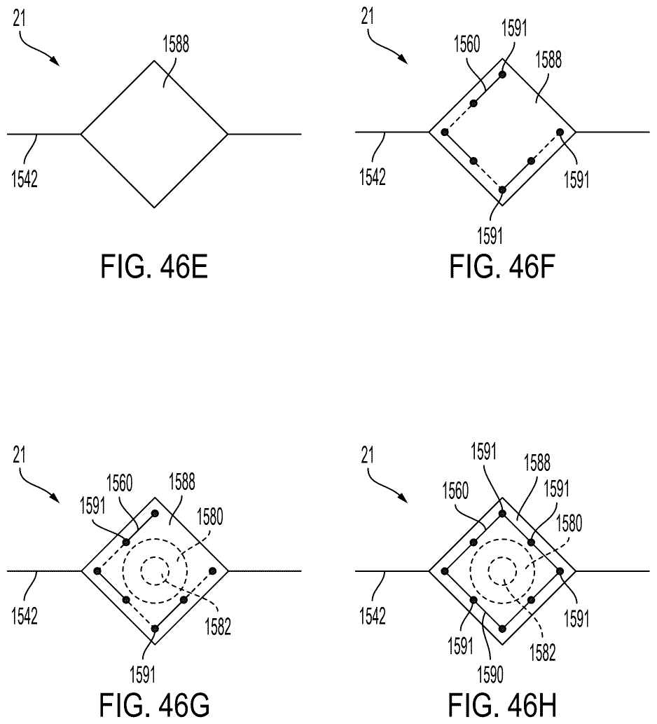

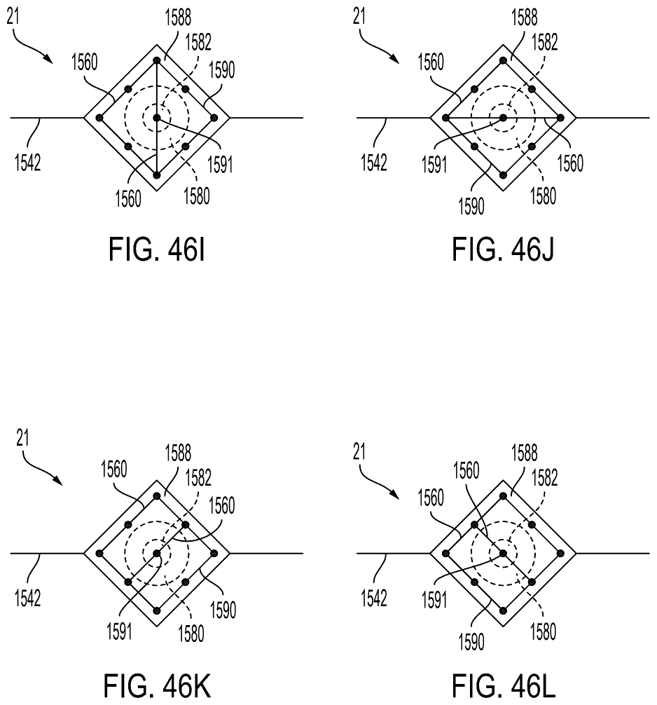

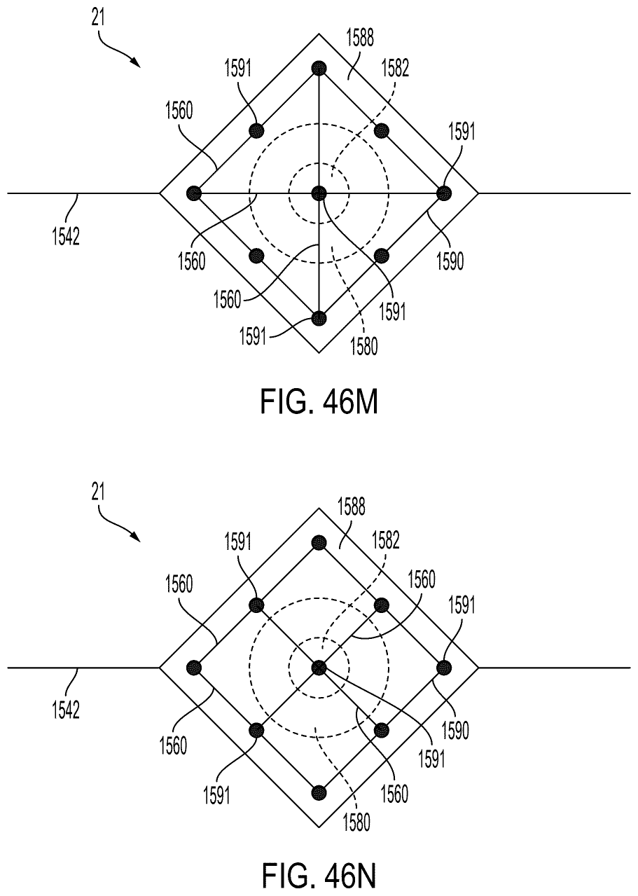

As shown in FIGS. 46A through 59, one or more radiopaque markers 1580 can be affixed to the frame 1500 of the docking station 10. In certain implementations, the radiopaque markers 1580 can be attached or affixed to the struts 1502 or junctions 1503 in the valve seat 18 of the frame 1500, with the radiopaque markers 1580 affixed to the frame 1500 in any suitable manner. For example, the radiopaque markers 1580 can be affixed to the frame 1500 by an adhesive, a suture, press fit, snap fit, or any other suitable means. The frame 1500 can include three or more radiopaque markers 1580 spaced circumferentially around the valve seat 18 to establish an annular plane through the valve seat 18 of the docking station 10. However, the frame 1500 can include fewer than three radiopaque markers 1580. In other implementations, the radiopaque markers 1580 can be attached or affixed to the impermeable material 21 as further described elsewhere in this disclosure, with the radiopaque markers 1580 attached or affixed at or near the struts 1502 or junctions 1503 of the frame 1500 or attached or affixed remotely from the struts 1502 and junctions 1503 of the frame 1500.

Additionally or alternatively, as shown in FIGS. 46A through 46D, one or more radiopaque markers 1580 can be included with the impermeable material 21 such that the one or more radiopaque markers 1580 are disposed within the valve seat 18 when the impermeable material 21 is attached to the frame 1500. The one or more radiopaque markers 1580 can be sewn onto, sewn into, encased by a pocket, or otherwise attached to the impermeable material 21 such that, when the impermeable material 21 is disposed on the frame 1500, the one or more radiopaque markers 1580 are disposed around the valve seat 18.

The radiopaque markers 1580 can be attached or affixed to the impermeable material 21 in various positions relative to the struts 1502 and junctions 1503 of the frame. In some implementations, the radiopaque markers 1580 can be attached or affixed at or near the struts 1502 or junctions 1503 of the frame 1500. In certain implementations, the radiopaque markers 1580 can be attached or affixed remotely from the struts 1502 and junctions 1503 of the frame 1500, such as a location in the central portions of cells 1504 of the frame. Positioning the radiopaque markers 1580 in the central portion of cells 1504 can provide certain technical advantages. One technical advantage is that the crimped profile of frame 1500 can be reduced as overlaps between the radiopaque marker 1580 with its associated attachment materials and the struts 1502 and junctions 1503 of the frame 1500 can be minimized. Another technical advantage is that physical contact between the radiopaque marker 1580 and the frame 1500 can be minimized, which can avoid material fatigue, degradation, and/or corrosion. A further technical advantage is that placement in the central portion of a cell 1504 can allow the radiopaque marker 1580 to move radially outwards to accommodate an expanding valve 29 within the frame 1500, which can reduce the physical contact between the valve 29 frame 712 and the frame 1500 and avoid interference with the proper function of the valve 29.

As shown in FIG. 46A, the one or more radiopaque markers 1580 can be sewn into the impermeable material 21 at or near the optional medial joint 1542 when the proximal portion 1520 is attached to the distal portion 1530. For example, the suture 1560 can be passed through the aperture 1582 to attach the radiopaque marker 1580 to the impermeable material 21. The one or more radiopaque markers 1580 can also be sewn onto the impermeable material 21 at or near the medial joint 1542 after the proximal portion 1520 has been attached to the distal portion 1530 or if the proximal portion 1520 is integrally formed with the distal portion 1530. The one or more radiopaque markers 1580 can be affixed to the outside of the impermeable material 21 such that the radiopaque markers do not interfere with the valve 29 when the valve 29 is deployed in the valve seat 18. However, the one or more radiopaque markers 1580 can also be affixed to the inside of the impermeable material 21.

As shown in FIGS. 46B through 46D, the one or more radiopaque markers 1580 can also be disposed in one or more pockets 1586 in or on the impermeable material 21. The one or more pockets can take a wide variety of different forms. The pockets can be formed from a patch of material or by any other manner of forming a pocket. For example, any manner that pockets are formed in clothing can be used on the impermeable material 21.

The one or more pockets 1586 can be sized and shaped to receive one of the radiopaque markers 1580. The pockets 1586 can be generally rectangular or diamond shaped. However, the pockets 1586 can also be triangular, circular, elliptical, or any other suitable shape. In one embodiment, the pockets 1586 extend radially outwardly from the remainder of the impermeable member 21. However, the pockets 1586 can alternatively extend radially inwardly from the remainder of the impermeable material 21. The pockets 1586 can be a part of or near the medial joint 1542 of the impermeable material 21. For example, the proximal and distal portions 1520, 1530 can be sized and shaped such that the pockets 1586 are formed when the proximal portion 1520 is attached to the distal portion 1530. The pockets 1586 can be formed from additional material or patch added to the proximal portion 1520 and/or the distal portion 1530 or can be formed from additional impermeable material 21 attached to an area defined by the proximal and/or distal portions 1520, 1530. The one or more pockets 1586 can be spaced around the impermeable material 21 at or near the medial joint 1542 such that the pockets 1586 are disposed around the circumference of the valve seat 18 of the docking station 10 when the impermeable material 21 is attached to the frame 1500.

The radiopaque markers 1580 can be disposed in, and secured in, the pockets 1586. In one embodiment, the radiopaque markers 1580 are disposed in the pockets 1586 before the impermeable material 21 is attached to the frame 1500. The pockets 1586 can then be covered by one or more pocket coverings 1588, the pocket can be stitched closed, and/or a stitch can be passed through the radiopaque marker to secure the marker in the pocket. The optional pocket coverings 1588 can be sized and shaped to cover the opening of the pockets 1586 and can comprise the same material as the impermeable material 21. The pocket coverings 1588 can be attached to the impermeable material 21 on the side of the impermeable material 21 opposite the frame 1500. The pocket coverings 1588 can be attached to the impermeable material 21 by one or more sutures 1560. The pockets 1586 can alternatively be formed by attaching the pocket coverings 1588 to the impermeable material 21 and thereby defining the pocket 1586 as the space between the pocket covering 1588 and the impermeable member 21.

Referring to FIGS. 46C and 46D, a suture 1560 can attach the pocket coverings 1588 to the impermeable material 21 around the outside of the pocket covering 1588 and can include a support stitch 1589 extending across the pocket covering 1588. The support stitch 1589 can provide radial force against the valve 29 when the valve 29 is deployed in the valve seat 18. The support stitch 1589 can be in line with or parallel to the medial joint 1542 (FIG. 46C) or can be perpendicular to the medial joint 1542 (FIG. 46D).

Referring to FIGS. 46E through 46N, the radiopaque markers 1580 with optional apertures 1582 can be disposed and secured in the pockets 1586 (not pictured, as the pocket 1586 is formed between the pocket covering 1588 and the impermeable member 21) of the impermeable member 21. The radiopaque markers 1580 can be secured in the pockets 1586 in a manner that can increase the securement of the radiopaque markers 1580 and can decrease the translational and rotational movement of the radiopaque markers 1580. As shown in FIGS. 46E and 46F, the pocket covering 1588 can be disposed on the impermeable material 21 and partially secured to the impermeable material 21 by a suture 1560. The suture 1560 can be stitched around a portion of the pocket covering 1588 to partially secure the pocket covering 1588 to the impermeable member 21 such that a portion of pocket covering 1588 is not secured to the impermeable member 21. The suture 1560 can be passed through the pocket covering 1588 and the impermeable member 21 at a plurality of through points 1591. The through points 1591 can be near the edges of the pocket covering 1588 and can substantially surround the perimeter of the pocket covering 1588. For example, the suture 1560 can be passed through the through points 1591 to surround three-fourths of the perimeter of the pocket covering 1588. In the illustrated embodiment, the suture 1560 is stitched through the through points 1591 by an in and out stitch. However, the suture 1560 can be stitched through the through points 1591 by any suitable stitch.

As shown in FIG. 46G, the radiopaque marker 1580 can then be placed in the pocket 1586 formed between the impermeable material 21 and the pocket covering 1588. The radiopaque marker 1580 can be positioned or oriented such that the aperture 1582 extends between the pocket covering 1588 and the impermeable member 21. As shown in FIG. 46H, the remainder of the pocket covering 1588 can be secured to the impermeable member 21 by the suture 1560. The suture 1560 can be passed through an additional through point 1591 such that the suture 1560 substantially surrounds the radiopaque marker 1580 near the edges of the pocket covering 1588. The suture 1560 can be passed through the pocket covering 1588 and the impermeable member 21 such that the suture 1560 passes through the initial through point 1591. Optionally, as shown in FIG. 46H, the suture 1560 can be stitched back through the through points 1591 to create an interlocking stitch 1590, similar to the stitch described in FIGS. 42E through 42I.

While the pocket covering 1588 has been described as partially stitched to the impermeable member 21 before the radiopaque marker 1580 is placed in the pocket 1586 the pocket covering 1588 can be attached to the impermeable member 21 in other ways. For example, the radiopaque marker 1580 can be disposed between the pocket covering 1588 and the impermeable member 21 and the pocket covering 1588 can then be stitched to the impermeable member 21.

Referring to FIGS. 46I through 46L, the radiopaque marker 1580 can be further secured in the pocket 1586 with a cross stitch. The suture 1560 can be stitched from one of the through points 1591 to a through point 1591 on the opposite side of the pocket covering 1588 and can also pass through a through point 1591 in the center of the pocket covering 1588. The center through point 1591 can be aligned with the aperture 1582 of the radiopaque marker 1580 such that the suture 1560 extends through the aperture 1582 of the radiopaque marker 1580. The additional stitch can be configured such that the suture 1560 extends vertically across the pocket covering 1588 (as shown in FIG. 46I), such that the suture 1560 extends horizontally across the pocket covering 1588 (as shown in FIG. 46J), and/or such that the suture 1560 extends diagonally across the pocket covering 1588 (as shown in FIGS. 46K and 46L).

As shown in FIGS. 46M and 46N, the radiopaque marker 1580 can be still further secured in the pocket 1586 with a second cross stitch. The second cross stitch of the suture 1560 can extend from one of the through points 1591 to a through point 1591 on the opposite side of the pocket covering 1588 and can also pass through the through point 1591 in the center of the pocket covering 1588. This second stitch through the center through point 1591 can be substantially perpendicular to the first cross stitch across the pocket covering 1588 which extends through the center through point 1591. As such, the suture 1560 can form a “+” or “X” shape across the pocket covering 1588. However, the suture 1560 can be stitched in any shape to secure the radiopaque marker 1580 in the pocket 1586 and can include more than two stitches extending through the center through point 1591.

While the frame 1500 has been described as having radiopaque markers 1580 disposed in the valve seat 18, the outer tube 4910 has been described as having radiopaque markers near the distal end of the outer tube 4910, and the connecting tube 4916 has been described as having radiopaque markers disposed along the shaft of the connecting tube 4916 for indicating the deployment of the frame 1500, the outer tube 4910, connecting tube 4916, frame 1500, and/or any other components of the delivery system can have any suitable configuration of portions of increased radiopacity or radiodensity which can provide an indication as to the amount of frame 1500 expansion or deployment prior to the release of the frame 1500. For example, the frame 1500 can include radiopaque markers 1580 at junctions 1503 distal to the valve seat 18 which, when aligned with the radiopaque markers of the catheter 3600 during deployment of the frame 1500, indicate the desired or maximum amount of frame 1500 expansion and/or deployment before the frame 1500 is released from the catheter 3600.

In the embodiments of FIGS. 46B through 46N, the pockets 1586 are formed between the impermeable material 21 and patches or pocket coverings 1588 attached (e.g., stitched or sutured) to the impermeable material around the radiopaque marker 1580. In other embodiments, according to another exemplary implementation of the present disclosure, the impermeable material 21 may include proximal and distal portions 1520, 1530 that are joined along overlapping inner axial portions to form one or more radiopaque marker retaining pockets, without requiring additional patch or pocket covering materials.

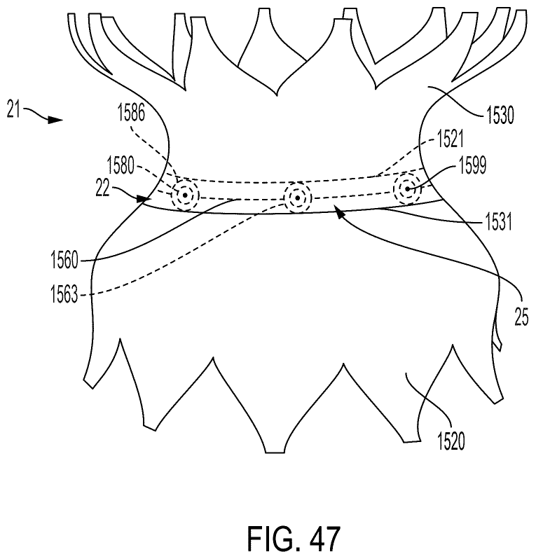

FIG. 47 illustrates an exemplary cover 21 for attachment to an expandable docking station frame (e.g., any of the exemplary expandable docking station frames described herein), for example, within an inner diameter of the docking station frame or surrounding an outer diameter of the docking station frame. The exemplary cover 21 includes proximal and distal portions 1520, 1530 having inner axial end portions 1521, 1531 that overlap to define a medial pocket region 22 in which one or more radiopaque markers 1580 are retained. In an exemplary embodiment, the proximal inner axial end portion 1521 extends radially inward of the distal inner axial end portion 1531, for example, to provide a downstream facing internal seam in the cover 21, minimizing blood flow through the seam.

In one such arrangement, one or more sutures may extend around the pocket region 22 to secure the proximal and distal portions 1520, 1530 together, and to secure the radiopaque markers 1580 within the pocket region 22. For example, the axial inner ends 1521, 1531 of each of the proximal and distal portions 1520, 1530 may be joined to the other of the proximal and distal portions along the circumferential suture 1560 around the circumference of the covering 21 to form a continuous annular pocket 25. The radiopaque markers 1580 may be circumferentially secured within the annular pocket 25, for example, by a stitch, suture, staple, adhesive, or other fastening element 1599 (e.g., through a central aperture in the radiopaque marker), to prevent migration of the radiopaque markers around the annular pocket. As another example, the cover may additionally or alternatively include a peripheral stitch or suture pattern 1563 around each radiopaque marker 1580 may form individual pockets 1586 around the circumference of the medial pocket region 22, securing the markers in desired locations (e.g., equally circumferentially spaced) and enclosing the markers in the cover 21.

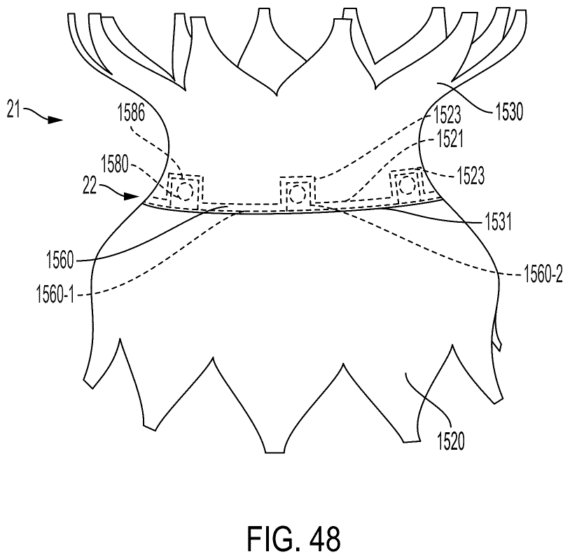

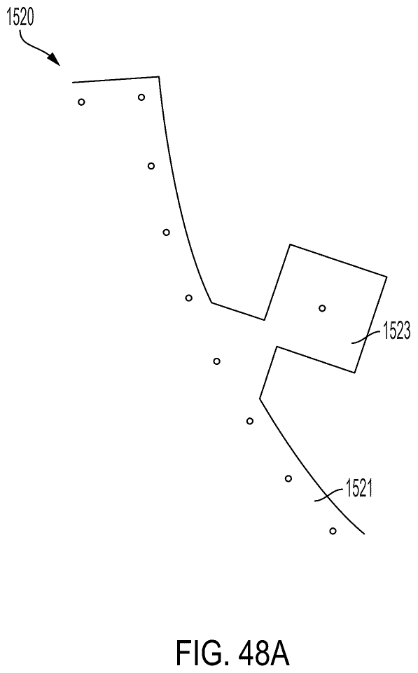

In other embodiments, the inner axial end portions 1521, 1531 of either or both of the proximal and distal cover portions 1520, 1530 may include flap portions that extend to form radiopaque marker retaining pockets when the inner axial end portions are joined, for example by stitches or sutures. FIG. 48 illustrates an exemplary impermeable frame covering 21 including proximal and distal portions 1520, 1530 having inner axial end portions 1521, 1531 that overlap to define a medial pocket region 22 in which one or more radiopaque markers 1580 are retained. In an exemplary embodiment, the proximal inner axial end portion 1521 extends radially inward of the distal inner axial end portion 1531, for example, to provide a downstream facing internal seam in the cover 21, minimizing blood flow through the seam from the interior of the docking station frame.

The proximal inner axial end portion 1521 includes axially extending flap portions 1523 that define discrete radiopaque marker retaining pockets 1586 when the proximal inner axial end portion is joined with the distal inner axial end portion 1531 (e.g., by stitches or sutures). In an exemplary arrangement, a suture 1560 includes a circumferential suture portion 1560-1 extending around the overlapping inner axial end portions 1521, 1531 of the proximal and distal portions 1520, 1530, and pocket suture portions 1560-2 extending around the peripheries of the flap portions 1523 (e.g., with one side of the periphery coinciding with the circumferential suture portion 1560-1) to form pockets 1586 retaining the radiopaque markers 1580. In the embodiment of FIG. 48, the flap portions 1523 are substantially rectangular to form rectangular pockets 1586. In other embodiments, other flap shapes may be utilized, including, for example, triangular, semicircular, or diamond-shaped, which may or may not include a peripheral side that coincides with the circumferential suture pattern. FIG. 48A illustrates an exemplary alternative embodiment of a cover portion 1520 including an inner axial end portion 1521 having a diamond-shaped flap portion 1523. As shown, the flap portion 1523 may be offset from the intended location of the circumferential suture portion, such that a peripheral pocket suture portion does not coincide with the circumferential suture portion.

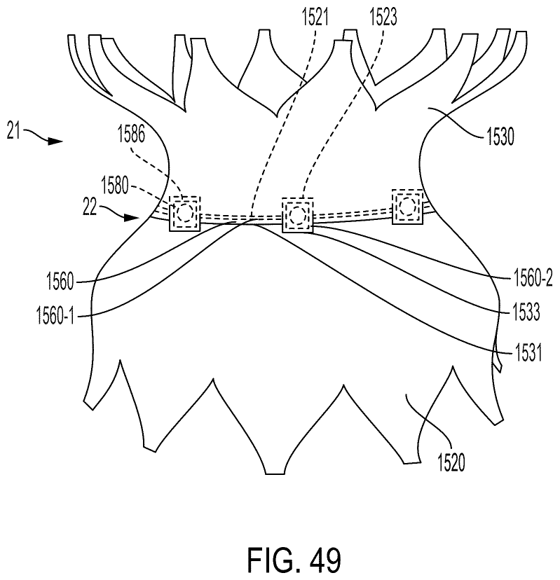

FIG. 49 illustrates an exemplary impermeable frame covering 21 including proximal and distal portions 1520, 1530 having inner axial end portions 1521, 1531 that overlap to define a medial pocket region 22 in which one or more radiopaque markers 1580 are retained. In an exemplary embodiment, the proximal inner axial end portion 1521 extends radially inward of the distal inner axial end portion 1531, for example, to provide a downstream facing internal seam in the cover 21, minimizing blood flow through the seam from the interior of the docking station frame.

The proximal inner axial end portion 1521 and the distal inner axial end portion 1531 each include axially extending flap portions 1523, 1533 that align to define discrete radiopaque marker retaining pockets 1586 when the proximal inner axial end portion is joined with the distal inner axial end portion (e.g., by stitches or sutures). In an exemplary arrangement, a circumferential suture portion 1560-1 extends around the overlapping inner axial end portions 1521, 1531 of the proximal and distal portions 1520, 1530, and peripheral stitch or suture 1560-2 extend around the peripheries of the aligned flap portions 1523, 1533 to form pockets 1586 retaining the radiopaque markers 1580, the pockets being substantially centered on the circumferential suture portion 1560-1. In the embodiment of FIG. 49, the flap portions 1523, 1533 are substantially rectangular to form rectangular pockets 1586. In other embodiments, other flap shapes may be utilized, including, for example, triangular or semicircular, which may or may not include a peripheral side that coincides with the circumferential suture pattern.

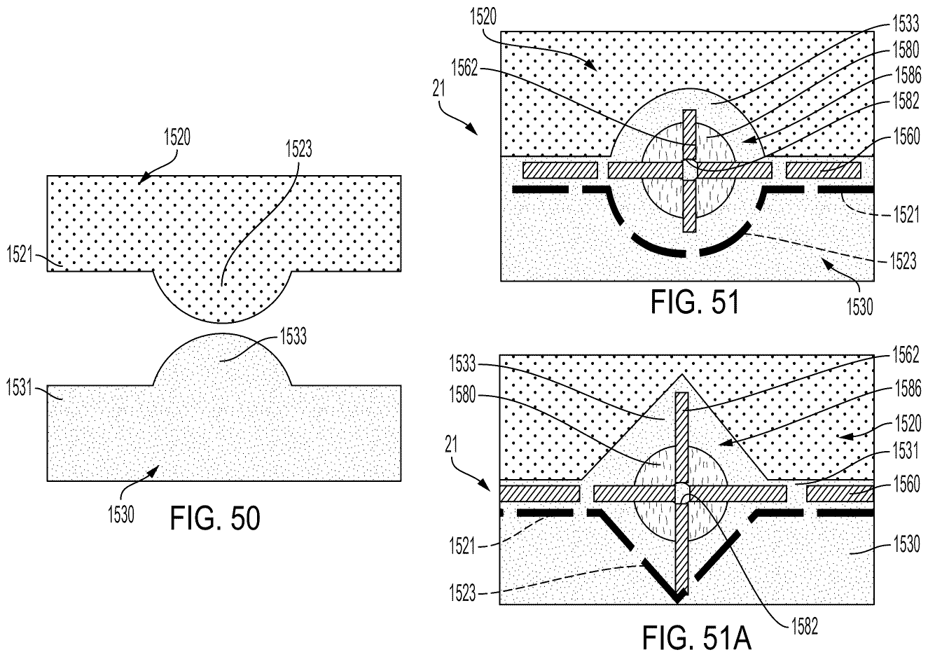

In other arrangements, aligned flaps may be joined the horizontal circumferential suture pattern and a crossing suture across the flap portions to hold the flap portions together and secure the radiopaque marker between the flap portions in a more loosely defined pocket, without a peripheral suture pattern. FIG. 50 illustrates axial inner portions 1521, 1531 of proximal (e.g., inflow) and distal (e.g., outflow) cover portions 1520, 1530 with axially extending flap portions 1523, 1533, prior to being joined for pocketed retention of a radiopaque marker. As shown in FIG. 51, when the flap portions 1523, 1533 are aligned and overlapped to form the pocket 1586, and the proximal and distal cover portions 1520, 1530 are joined to form the cover 21, a circumferential (e.g., horizontal) suture 1560, used to join the proximal and distal cover portions, extends across the pocket and through an apertured radiopaque marker 1580 inserted between the flap portions. A crossing suture pattern 1562 (e.g., vertical or perpendicular to the circumferential suture pattern 1561) extends between axial edges of the flap portions 1523, 1533, through the aperture 1582 of the radiopaque marker 1580 and may be knotted at the ends, securing the edges of the flap portions against the adjoining material and reinforcing attachment of the radiopaque marker 1580 within the pocket 1586. In other embodiments, peripheral suture patterns may be added around the pockets 1586 to form a more substantially enclosed pocket around the radiopaque marker 1580.

In the embodiment of FIGS. 50 and 51, the flap portions 1523, 1533 are semicircular, joining to form a substantially circular pocket 1586. In other embodiments, other flap shapes may be used to form other suitable pocket shapes. For example, FIG. 51A illustrates a cover 21 including proximal and distal cover portions 1520, 1530 having triangular flap portions 1523, 1533 that overlap to form a substantially diamond shaped pocket 1586.

Many different suture/stitch pattern arrangements and methods may be used to secure the radiopaque marker 1580 in the pocket 1586 formed by the overlapping flap portions 1523, 1533 of the proximal and distal cover portions 1520, 1530 (e.g., the overlapping flap portions of the embodiment of FIG. 51A). The stitches or suture segments described herein may include any suitable stitch or suture arrangement including, for example, in and out stitches. In some embodiments, a crossing suture pattern may be stitched through the radiopaque marker (e.g., through an aperture in the radiopaque marker) to anchor the radiopaque marker in the pocket. In some such embodiments, this crossing suture pattern may be formed from a separate suture (i.e., separate from the circumferential suture). In other embodiments the crossing suture pattern may be part of the same suture forming the circumferential suture pattern. In some embodiments, the suture may be formed from first and second suture patterns that interlock with and/or backfill with each other to reinforce the suture. In some embodiments, the first and second suture patterns may be substantially continuous with each other (e.g., formed from the same suture). In other embodiments, the first and second suture patters may be formed from separate sutures/threads.

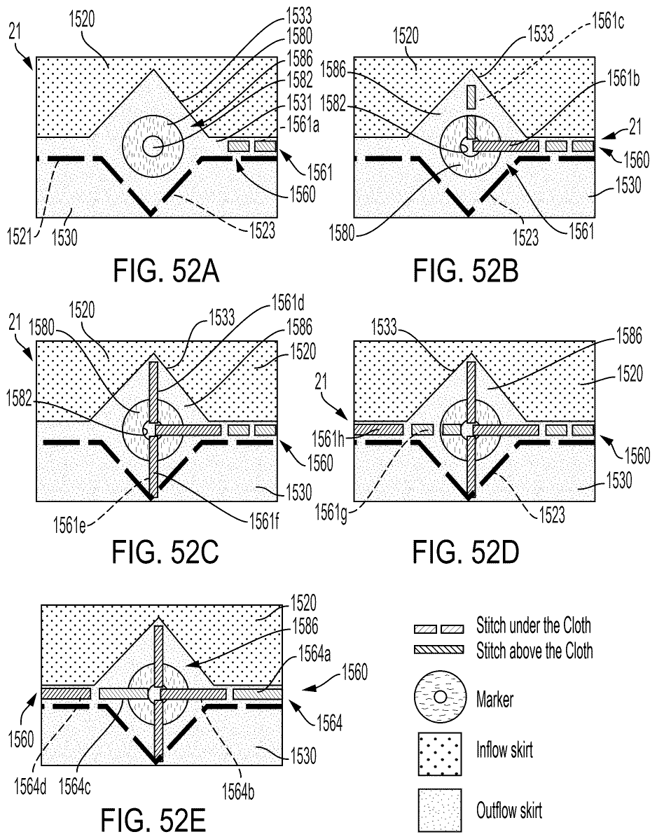

FIGS. 52A-52E illustrate steps of an exemplary suturing method for securing a radiopaque marker 1580 in a flap-defined pocket 1586 (e.g., diamond-shaped, as shown) of the cover 21. As shown in FIG. 52A, an axial end portion 1521 of a proximal (e.g., inflow) cover portion 1520 underlays an axial end portion 1531 of a distal (e.g., outflow) cover portion 1530 with a radiopaque marker 1580 disposed between aligned flap portions 1523, 1533 forming a pocket region 1586. A first segment or stitch 1561a of a first suture pattern 1561 of a suture 1560 extends to a first (e.g., right, as shown) circumferential end of the pocket region 1586, for example, under or radially inward of the proximal cover portion 1520. As shown in FIG. 52B, a second segment or stitch 1561b of the first suture pattern 1561 is sewed horizontally, for example, over or radially outward of the distal cover portion 1530 to the center of the radiopaque marker 1580, and a third segment or stitch 1561c is sewed through a central aperture 1582 in the radiopaque marker, and vertically, for example, under or radially inward of the proximal cover portion 1520 to an axial end of the distal cover flap portion 1533. As shown in FIG. 52C, a fourth segment or stitch 1561d is sewed through the proximal and distal cover portions 1520, 1530 and vertically, for example, over or radially outward of the distal cover portion 1530, to the center of the radiopaque marker 1580, and interlocking (e.g., through a common through point) with the third stitch 1561c. A fifth segment or stitch 1561e is sewed through the central aperture 1582 of the radiopaque marker 1580, and vertically, for example, under or radially inward of the proximal cover portion 1520 to an axial end of the proximal cover flap portion 1523, and a sixth segment or stitch 1561f is sewed through the proximal and distal cover portions 1520, 1530 and vertically, for example, over or radially outward of the distal cover portion 1530, to the center of the radiopaque marker 1580, and interlocking (e.g., through a common through point) with the fifth stitch 1561e. As shown in FIG. 52D, a seventh segment or stitch 1561g is sewed through the central aperture 1582 of the radiopaque marker 1580 and horizontally, for example, under or radially inward of the proximal cover portion 1520, to a second (e.g., left, as shown) circumferential end of the pocket region 1586. An eighth segment or stitch 1561h is sewed horizontally, for example, over or radially outward of the distal cover portion 1530, away from the pocket region 1586, to continue the first circumferential suture pattern 1561. As shown in FIG. 52E, a first segment or stitch 1564a of a second circumferential suture pattern 1564 of the suture 1560 extends to the first circumferential end of the pocket region 1586, for example, over or radially outward of the distal cover portion 1530, and interlocking (e.g., through a common through point) with the first segment 1561a of the first circumferential suture pattern 1561. A second segments 1564b of the second circumferential suture pattern 1564 is sewed horizontally, for example, under or radially inward of the proximal cover portion 1520, and interlocking (e.g., through a common through point) with the second segment 1561b of the first circumferential suture pattern 1561. A third segments 1564c is sewed horizontally, for example, over or radially outward of the distal cover portion 1530, and interlocking (e.g., through a common through point) with the seventh segment 1561g of the first circumferential suture pattern 1561. A fourth segment 1564d is sewed horizontally, for example, under or radially inward of the proximal cover portion 1520, away from the pocket region 1586, to continue the second circumferential suture pattern 1564 (e.g., for continued interlocking with the first circumferential suture pattern 1561).

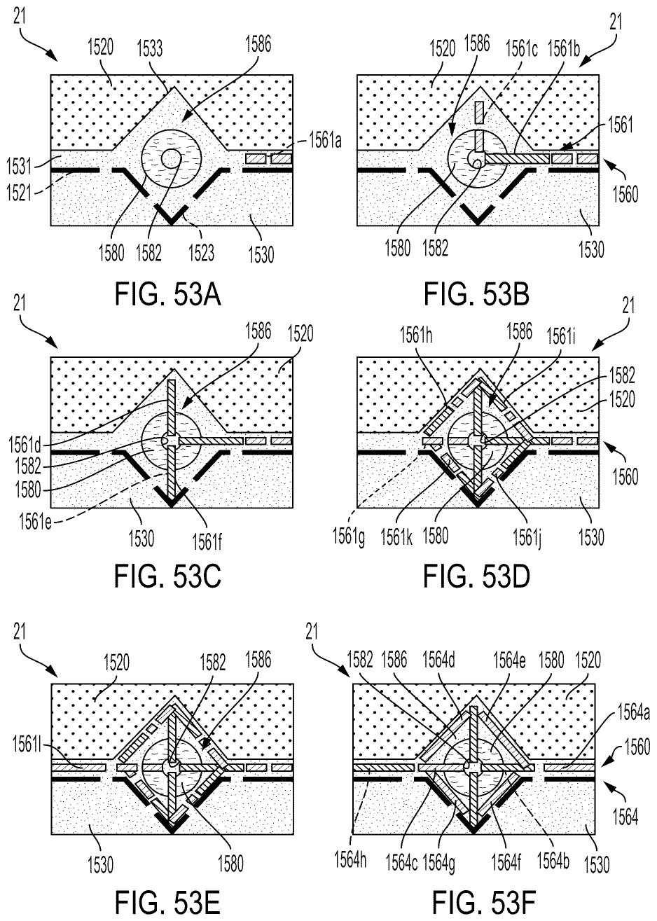

FIGS. 53A-53F illustrate steps of another exemplary suturing method for securing a radiopaque marker 1580 in a flap-defined pocket 1586 (e.g., diamond-shaped, as shown) of the cover 21. As shown in FIG. 53A, an axial end portion 1521 of a proximal (e.g., inflow) cover portion 1520 underlays an axial end portion 1531 of a distal (e.g., outflow) cover portion 1530 with a radiopaque marker 1580 disposed between aligned flap portions 1523, 1533 forming a pocket region 1586. In the illustrated example, as shown in FIGS. 53A-53D, the first through seventh segments or stitches 1561a-g of a first circumferential suture pattern 1561 of a suture 1560 substantially match the first through seventh segments or stitches of the first suture pattern of FIGS. 52A-52D. As further shown in FIG. 53D, eighth, ninth, tenth and eleventh segments 1561h-k include additional stitches (e.g., two or more per side of the pocket region) interlaced through the proximal and distal cover portions 1520, 1530 and around the perimeter of the pocket region 1586, and a twelfth segment or stitch 1561l is sewed horizontally, for example, under or radially inward of the proximal cover portion 1520, away from the pocket region 1586, to continue the circumferential suture pattern 1561. As shown in FIG. 53F, a first segment or stitch 1564a of a second circumferential suture pattern 1564 of the suture 1560 extends to the first circumferential end of the pocket region 1586, for example, over or radially outward of the distal cover portion 1530, and interlocking (e.g., through a common through point) with the first segment 1561a of the first circumferential suture pattern 1561. A second segments 1564b is sewed horizontally, for example, under or radially inward of the proximal cover portion 1520, and interlocking (e.g., through a common through point) with the second segment 1561b of the first circumferential suture pattern 1561. A third segments 1564c is sewed horizontally, for example, over or radially outward of the distal cover portion 1530, and interlocking (e.g., through a common through point) with the seventh segment 1561g of the first circumferential suture pattern 1561. Fourth, fifth, sixth, and seventh segments 1564d-g of the second circumferential suture pattern 1564 include additional stitches (e.g., two or more per side of the pocket region) interlaced through the proximal and distal cover portions 1520, 1530 and around the perimeter of the pocket region 1586, either interlocking with or merely backfilling the eighth, ninth, tenth and eleventh segments 1561h-k of the first circumferential suture pattern 1561. An eighth segment 1564h of the second circumferential suture pattern 1564 is sewed horizontally, for example, under or radially inward of the proximal cover portion 1520, away from the pocket region 1586, to continue the second circumferential suture pattern 1564 (e.g., for continued interlocking with the first circumferential suture pattern 1561).

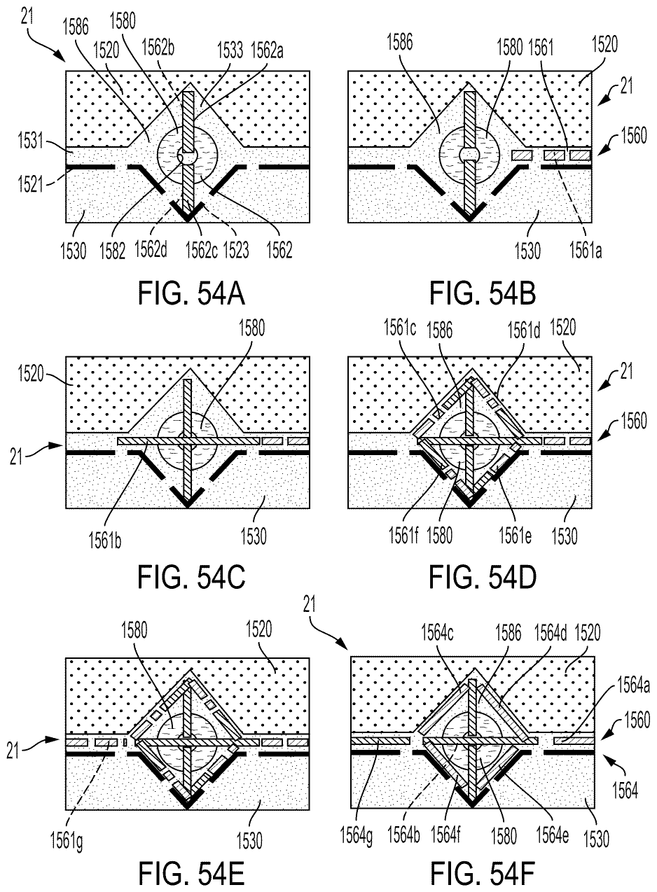

FIGS. 54A-54F illustrate steps of another exemplary suturing method for securing a radiopaque marker 1580 in a flap-defined pocket 1586 (e.g., diamond-shaped, as shown) of the cover 21. As shown in FIG. 54A, an axial end portion 1521 of a proximal (e.g., inflow) cover portion 1520 underlays an axial end portion 1531 of a distal (e.g., outflow) cover portion 1530 with a radiopaque marker 1580 disposed between aligned flap portions 1523, 1533 forming a pocket region 1586. Interlocking first, second, third and fourth vertical segments or stitches 1562a-d of a crossing suture pattern 1562 extend between axial ends of the proximal and distal cover flap portions 1523, 1533 and the radiopaque marker aperture 1582 and are knotted to secure the radiopaque marker 1580 in place. As shown in FIG. 54B, a first segment or stitch 1561a of a first circumferential suture pattern 1561 of a suture 1560 extends to a first (e.g., right, as shown) circumferential end of the pocket region 1586, for example, under or radially inward of the proximal cover portion 1520. As shown in FIG. 54C, a second segment or stitch 1561b is sewed horizontally, for example, over or radially outward of the distal cover portion 1530 across the pocket region 1586 to a second (e.g., left, as shown) circumferential end of the pocket region. As shown in FIG. 54D, third, fourth, fifth and sixth segments 1561c-f include additional stitches (e.g., two or more per side of the pocket region) interlaced through the proximal and distal cover portions 1520, 1530 and around the perimeter of the pocket region 1586. As shown in FIG. 54E, a seventh segment or stitch 1561g is sewed horizontally, for example, under or radially inward of the proximal cover portion 1520, away from the pocket region 1586, to continue the circumferential suture pattern 1561. As shown in FIG. 54F, a first segment or stitch 1564a of a second circumferential suture pattern 1564 of the suture 1560 extends to the first circumferential end of the pocket region 1586, for example, over or radially outward of the distal cover portion 1530, and interlocking (e.g., through a common through point) with the first segment 1561a of the first circumferential suture pattern 1561. A second segments 1564b of the second circumferential suture pattern 1564 is sewed horizontally, for example, under or radially inward of the proximal cover portion 1520 across the pocket region 1586 to the second circumferential end of the pocket region and interlocking (e.g., through a common through point) with the second segment 1561b of the first circumferential suture pattern 1561. Third, fourth, fifth, and sixth segments 1564c-f of the second circumferential suture pattern 1564 include additional stitches (e.g., two or more per side of the pocket region) interlaced through the proximal and distal cover portions 1520, 1530 and around the perimeter of the pocket region 1586, either interlocking with or merely backfilling the third, fourth, fifth and sixth segments 1561c-f of the first circumferential suture pattern 1561. A seventh segment 1564g of the second circumferential suture pattern 1564 is sewed horizontally, for example, over or radially outward of the distal cover portion 1530, away from the pocket region 1586, to continue the second circumferential suture pattern 1564 (e.g., for continued interlocking with the first circumferential suture pattern 1561).

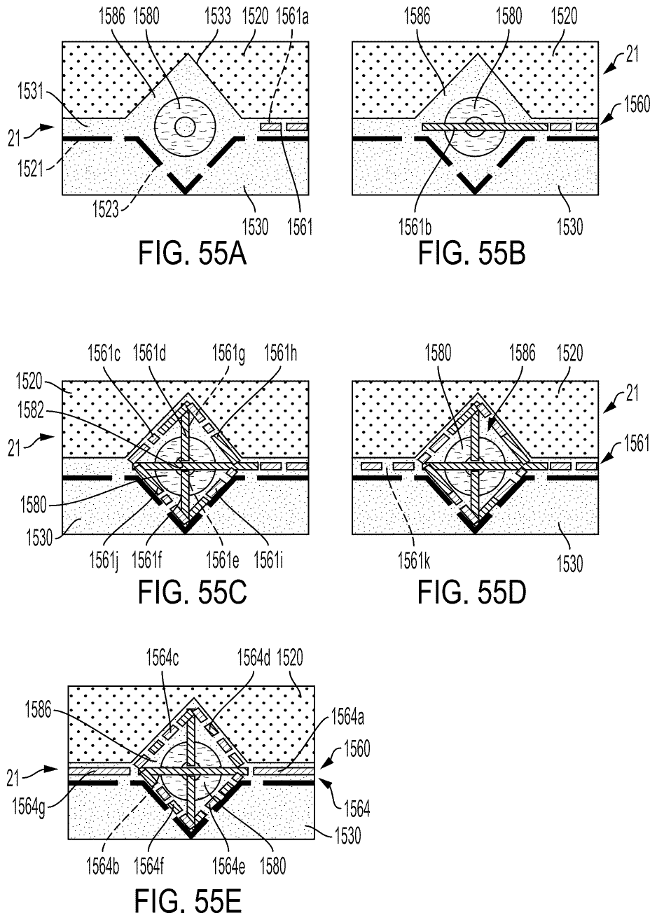

FIGS. 55A-55E illustrate steps of another exemplary suturing method for securing a radiopaque marker 1580 in a flap-defined pocket 1586 (e.g., diamond-shaped, as shown) of the cover 21. As shown in FIG. 55A, an axial end portion 1521 of a proximal (e.g., inflow) cover portion 1520 underlays an axial end portion 1531 of a distal (e.g., outflow) cover portion 1530 with a radiopaque marker 1580 disposed between aligned flap portions 1523, 1533 forming a pocket region 1586. A first segment or stitch 1561a of a first circumferential suture pattern 1561 of a suture 1560 extends to a first (e.g., right, as shown) circumferential end of the pocket region 1586, for example, under or radially inward of the proximal cover portion 1520. As shown in FIG. 55B, a second segment or stitch 1561b is sewed horizontally, for example, over or radially outward of the distal cover portion 1530 across the pocket region 1586 to a second (e.g., left, as shown) circumferential end of the pocket region. As shown in FIG. 55C, a third segment 1561c includes additional stitches (e.g., two or more stitches) interlaced through the proximal and distal cover portions 1520, 1530 along a first perimeter side of the pocket region 1586. Interlocking (e.g., through common through points) fourth, fifth, sixth, and seventh segments 1561d-g extend vertically from the axial end of the distal cover flap portion 1533, through the radiopaque marker aperture 1582, to the axial end of the proximal cover flap portion 1523, back through the radiopaque marker aperture, and back to the axial end of the distal cover flap portion. Eighth, ninth, and tenth segments 1561h-j include additional stitches (e.g., two or more per side of the pocket region) interlaced through the proximal and distal cover portions 1520, 1530 and around the second, third, and fourth sides of the perimeter of the pocket region 1586. As shown in FIG. 55D, an eleventh segment or stitch 1561k is sewed horizontally, for example, under or radially inward of the proximal cover portion 1520, away from the pocket region 1586, to continue the circumferential suture pattern 1561. As shown in FIG. 55E, a first segment or stitch 1564a of a second circumferential suture pattern 1564 of the suture 1560 extends to the first circumferential end of the pocket region 1586, for example, over or radially outward of the distal cover portion 1530, and interlocking (e.g., through a common through point) with the first segment 1561a of the first circumferential suture pattern 1561. A second segments 1564b of the second circumferential suture pattern 1564 is sewed horizontally, for example, under or radially inward of the proximal cover portion 1520 across the pocket region 1586 to the second circumferential end of the pocket region and interlocking (e.g., through a common through point) with the second segment 1561b of the first circumferential suture pattern 1561. Third, fourth, fifth, and sixth segments 1564c-f of the second circumferential suture pattern 1564 include additional stitches (e.g., two or more per side of the pocket region) interlaced through the proximal and distal cover portions 1520, 1530 and around the perimeter of the pocket region 1586, either interlocking with or merely backfilling the third, eighth, ninth, and tenth segments 1561c, 1561h-j of the first circumferential suture pattern 1561. A seventh segment 1564g of the second circumferential suture pattern 1564 is sewed horizontally, for example, over or radially outward of the distal cover portion 1530, away from the pocket region 1586, to continue the second circumferential suture pattern 1564 (e.g., for continued interlocking with the first circumferential suture pattern 1561).

FIGS. 56A-56E illustrate steps of another exemplary suturing method for securing a dual apertured radiopaque marker 1580 in a flap-defined pocket 1586 (e.g., diamond-shaped, as shown) of the cover 21. As shown in FIG. 56A, an axial end portion 1521 of a proximal (e.g., inflow) cover portion 1520 underlays an axial end portion 1531 of a distal (e.g., outflow) cover portion 1530 with a radiopaque marker 1580 disposed between aligned flap portions 1523, 1533 forming a pocket region 1586. Interlocking (e.g., through common through points) first, second, third, fourth, and fifth vertical segments or stitches 1562a-e of a crossing suture pattern 1562 extend between axial ends of the proximal and distal cover flap portions 1523, 1533 and the radiopaque marker apertures 1582-1, 1582-2 and are knotted to secure the radiopaque marker 1580 in place. In the illustrated example, the crossing pattern stitches 1562a-e extend from the first radiopaque marker aperture 1582-1 to the axial end of the distal cover flap portion 1533, back to the first radiopaque marker aperture, to the second radiopaque marker aperture 1582-2, to the axial end of the proximal cover flap portion 1523, and back to the second radiopaque marker aperture. As shown in FIG. 56B, a first segment or stitch 1561a of a first circumferential suture pattern 1561 of a suture 1560 extends to a first (e.g., right, as shown) circumferential end of the pocket region 1586, for example, under or radially inward of the proximal cover portion 1520. As shown in FIG. 56C, second, third, fourth and fifth segments 1561b-e include additional stitches (e.g., two or more per side of the pocket region) interlaced through the proximal and distal cover portions 1520, 1530 and around the perimeter of the pocket region 1586, and sixth and seventh segments 1561f-g include additional stitches (e.g., two or more per side of the pocket region) interlocking (e.g., through common through points) with the fifth and fourth segment stitches, respectively, for interlocking reinforcement of the distal (outflow) sides of the pocket region 1586. As shown in FIG. 56D, an eighth segment or stitch 1561h is sewed horizontally, for example, over or radially outward of the distal cover portion 1530, away from the pocket region 1586, to continue the circumferential suture pattern 1561. As shown in FIG. 56E, a first segment or stitch 1564a of a second circumferential suture pattern 1564 of the suture 1560 extends to the first circumferential end of the pocket region 1586, for example, over or radially outward of the distal cover portion 1530, to interlock (e.g., through a common through point) with the first segment 1561a of the first circumferential suture pattern 1561. Second and third segments 1564b-c of the second circumferential suture pattern 1564 include additional stitches (e.g., two or more per side of the pocket region) interlocking (e.g., through common through points) with the second and third segment stitches 1561b-c of the first circumferential suture pattern 1561, respectively, for interlocking reinforcement of the proximal (inflow) sides of the pocket region 1586. A fourth segment 1564d is sewed horizontally, for example, under or radially inward of the proximal cover portion 1520, away from the pocket region 1586, to continue the second circumferential suture pattern 1564 (e.g., for continued interlocking with the first circumferential suture pattern 1561).

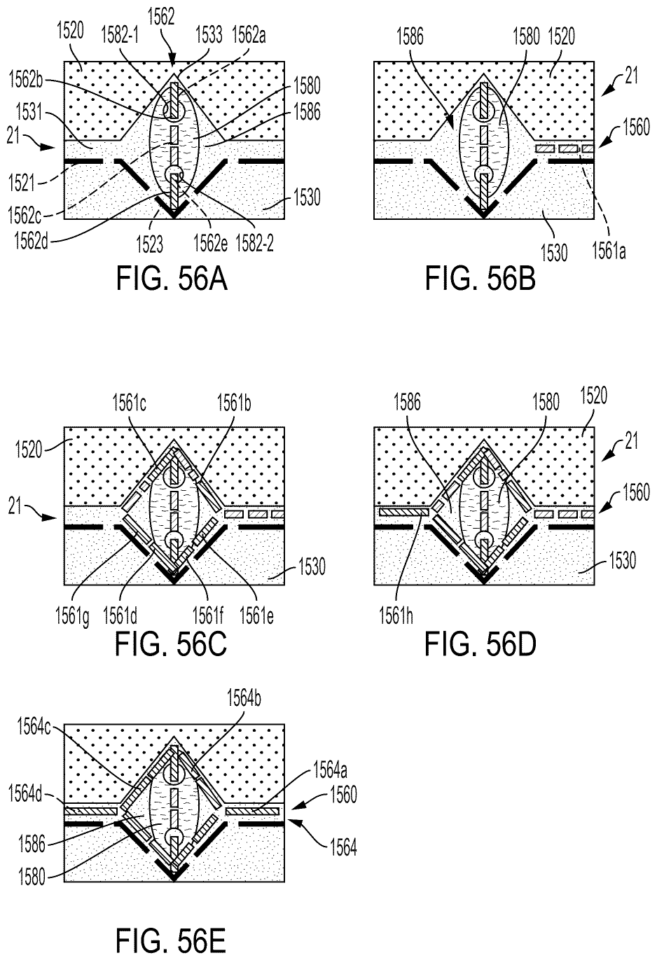

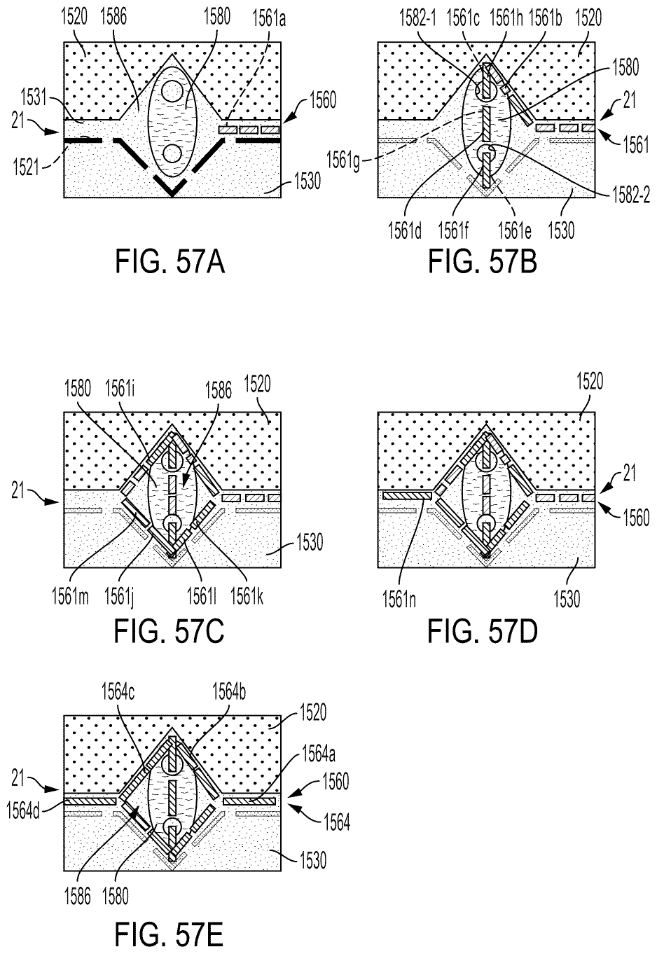

FIGS. 57A-57E illustrate steps of another exemplary suturing method for securing a radiopaque marker 1580 in a flap-defined pocket 1586 (e.g., diamond-shaped, as shown) of the cover 21. As shown in FIG. 57A, an axial end portion 1521 of a proximal (e.g., inflow) cover portion 1520 underlays an axial end portion 1531 of a distal (e.g., outflow) cover portion 1530 with a radiopaque marker 1580 disposed between aligned flap portions 1523, 1533 forming a pocket region 1586. A first segment or stitch 1561a of a first circumferential suture pattern 1561 of a suture 1560 extends to a first (e.g., right, as shown) circumferential end of the pocket region 1586, for example, under or radially inward of the proximal cover portion 1520. As shown in FIG. 57B, a second segment or stitch 1561b includes additional stitches (e.g., two or more stitches) interlaced through the proximal and distal cover portions 1520, 1530 along a first perimeter side of the pocket region 1586. Interlocking (e.g., through common through points) third, fourth, fifth, sixth, seventh, and eighth vertical segments or stitches 1561c-h extend from the axial end of the distal cover flap portion 1533, lacing through the radiopaque marker apertures 1582-1, 1582-2, to the axial end of the proximal cover flap portion 1523, back through the radiopaque marker apertures, and back to the axial end of the distal cover flap portion. As shown in FIG. 57C, ninth, tenth, and eleventh segments 1561i-k include additional stitches (e.g., two or more per side of the pocket region) interlaced through the proximal and distal cover portions 1520, 1530 and around the second, third, and fourth sides of the perimeter of the pocket region 1586, and twelfth and thirteenth segments 1561l-m include additional stitches (e.g., two or more per side of the pocket region) interlaced through the proximal and distal cover portions 1520, 1530 and back around the fourth and third sides of the perimeter of the pocket region 1586, interlocking (e.g., through common through points) with the eleventh and tenth segments 1561k, 1561j, respectively. As shown in FIG. 57D, a fourteenth segment or stitch 1561n is sewed horizontally, for example, over or radially outward of the distal cover portion 1530, away from the pocket region 1586, to continue the circumferential suture pattern 1561. As shown in FIG. 57E, a first segment or stitch 1564a of a second circumferential suture pattern 1564 of the suture 1560 extends to the first circumferential end of the pocket region 1586, for example, over or radially outward of the distal cover portion 1530, to interlock (e.g., through common through points) with the first segment 1561a of the first circumferential suture pattern 1561, for interlocking reinforcement of the distal (outflow) sides of the pocket region 1586. Second and third segments 1564b-c of the second circumferential suture pattern 1564 include additional stitches (e.g., two or more per side of the pocket region) interlocking with the second and ninth segments 1561b, 1561i of the first circumferential suture pattern 1561, respectively, for interlocking reinforcement of the proximal (inflow) sides of the pocket region 1586. A fourth segment 1564d of the second circumferential suture pattern 1564 is sewed horizontally, for example, under or radially inward of the proximal cover portion 1520, away from the pocket region 1586, to continue the second circumferential suture pattern 1564 (e.g., for continued interlocking with the first circumferential suture pattern 1561).

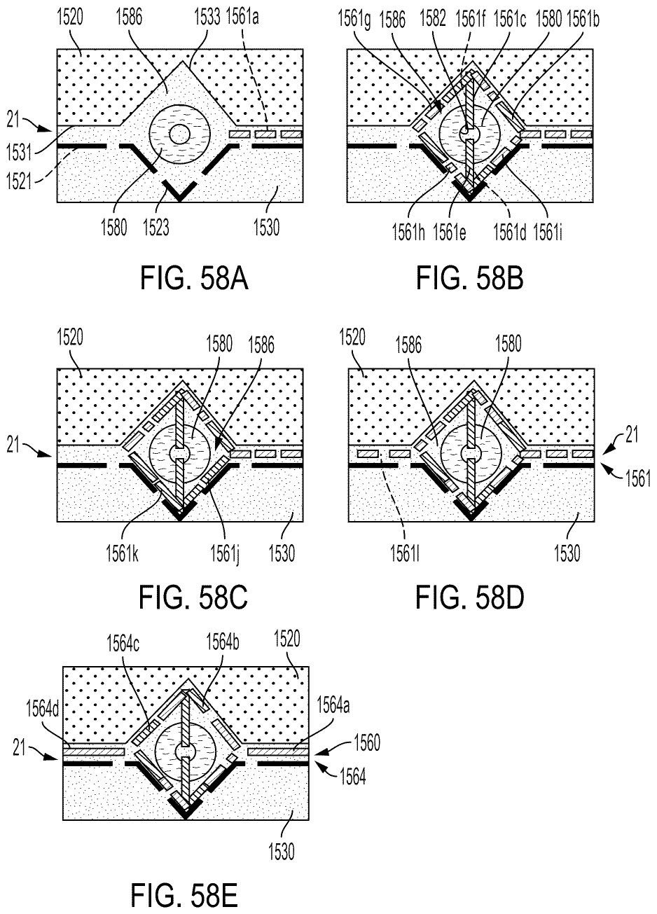

FIGS. 58A-58E illustrate steps of another exemplary suturing method for securing a radiopaque marker 1580 in a flap-defined pocket 1586 (e.g., diamond-shaped, as shown) of the cover 21. As shown in FIG. 58A, an axial end portion 1521 of a proximal (e.g., inflow) cover portion 1520 underlays an axial end portion 1531 of a distal (e.g., outflow) cover portion 1530 with a radiopaque marker 1580 disposed between aligned flap portions 1523, 1533 forming a pocket region 1586. A first segment or stitch 1561a of a first circumferential suture pattern 1561 of a suture 1560 extends to a first (e.g., right, as shown) circumferential end of the pocket region 1586, for example, under or radially inward of the proximal cover portion 1520. As shown in FIG. 58B, a second segment or stitch 1561b includes additional stitches (e.g., two or more stitches) interlaced through the proximal and distal cover portions 1520, 1530 along a first perimeter side of the pocket region 1586. Interlocking (e.g., through common through points) third, fourth, fifth, and sixth vertical segments or stitches 1561c-f extend from the axial end of the distal cover flap portion 1533, lacing through the radiopaque marker aperture, to the axial end of the proximal cover flap portion 1523, back through the radiopaque marker aperture, and back to the axial end of the distal cover flap portion. Seventh, eighth and ninth segments 1561g-i include additional stitches (e.g., two or more per side of the pocket region) interlaced through the proximal and distal cover portions 1520, 1530 and around the second, third, and fourth sides of the perimeter of the pocket region 1586. As shown in FIG. 58C, tenth and eleventh segments 1561j-k include additional stitches (e.g., two or more per side of the pocket region) interlaced through the proximal and distal cover portions 1520, 1530 and back around the fourth and third sides of the perimeter of the pocket region 1586, backfilling or interlocking with the ninth and eighth segments 1561i, 1561h, respectively. As shown in FIG. 58D, a twelfth segment or stitch 1561l is sewed horizontally, for example, under or radially inward of the proximal cover portion 1520, away from the pocket region 1586, to continue the circumferential suture pattern 1561. As shown in FIG. 58E, a first segment or stitch 1564a of a second circumferential suture pattern 1564 of the suture 1560 extends to the first circumferential end of the pocket region 1586, for example, over or radially outward of the distal cover portion 1530, to interlock (e.g., through a common through point) with the first segment 1561a of the first circumferential suture pattern 1561. Second and third segments 1564b-c of the second circumferential suture pattern 1564 include additional stitches (e.g., two or more per side of the pocket region) interlaced with the second and seventh segments 1561b, 1561g of the first circumferential suture pattern 1561, respectively. A fourth segment 1564d of the second circumferential suture pattern 1564 is sewed horizontally, for example, under or radially inward of the proximal cover portion 1520, away from the pocket region 1586, to continue the second circumferential suture pattern 1564 (e.g., for continued interlocking with the first circumferential suture pattern 1561).

According to another aspect of the present disclosure, an expandable docking station may be provided with an expandable frame having a cover including a waist restricting circumferential central band (e.g., one or more sutures, fabric, metal, polymer, a biocompatible tape, or other relatively unexpandable materials known in the art) having unreinforced or weakened (e.g., non-interlocking) portions that are frangible, for example, by expansion of a noncompliant balloon or other such expansion tool inserted into the frame, to selectively expand a waist portion of the docking station to provide an enlarged seating area for installation of a prosthetic device (e.g., a prosthetic valve). In one such arrangement, an original prosthetic valve expanded (e.g., by a self-expanding valve frame or balloon expansion of the valve) into seating engagement with the valve seat may be replaced (e.g., due to inadequate performance of the prosthetic valve) by insertion of the expansion tool into the docking station and through the prosthetic valve (e.g., between leaflets of the prosthetic valve), and plastic expansion of the prosthetic valve and docking station waist portion, breaking the frangible portions of the docking station suture pattern. The expanded prosthetic valve and docking station waist portion provide a replacement valve seat, within the original prosthetic valve, which is sized to accommodate a replacement prosthetic valve, which may be inserted into, and expanded into seating engagement with, the replacement valve seat. In other arrangements, the frangible waist restricting central band may provide for a valve seat having a first diameter accommodating seating engagement with a first, smaller diameter prosthetic valve expanded into the waist portion of the docking station, with frangible portions of the central band being breakable to provide a larger valve seat having a second diameter accommodating seating engagement with a second, larger diameter prosthetic valve expanded into the waist portion of the docking station.

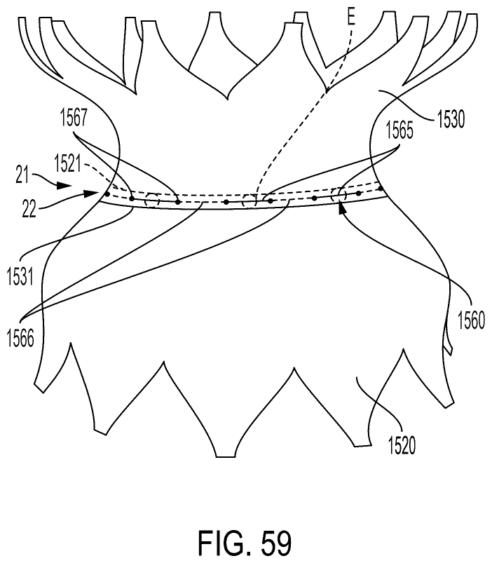

FIG. 59 illustrates an exemplary impermeable frame cover 21 including a medial waist portion 22 having a band or suture 1560 that extends along the medial waist portion 22 to provide a waist restricting valve seat on the inner periphery of the cover 21. While the cover may be a single piece of material (e.g., single fabric sheet) or multiple pieces of material attached at different locations, in the illustrated embodiment, the cover 21 includes proximal and distal portions 1520, 1530 having inner axial end portions 1521, 1531 that overlap at the medial waist portion 22 (e.g., with the proximal inner axial end portion 1521 extending radially inward of the distal inner axial end portion 1531) and are joined together by the suture 1560, which is stitched with the proximal and distal cover portions 1520, 1530 to secure the proximal and distal portions together.

The band or suture 1560 includes first reinforced portions 1565 (e.g., interlocking sutures extending through common through points) spaced apart by second, unreinforced or weakened frangible portions 1566 (e.g., non-interlocking sutured portions). In some embodiments, the reinforced portions 1565 may be separated from the frangible portions 1566 by suture knots or other such reinforcing junctions (e.g., clips, fasteners) 1567 to provide additional reinforcement. The proportion of frangible portions 1566 to reinforced portions 1565 of the band/suture may be selected to provide for a desired degree of radial expansion of the cover 21 (and the frame on which the cover is installed) when the frangible portions 1566 are broken, for example, by expansion of a noncompliant balloon or other tool within the cover and frame.

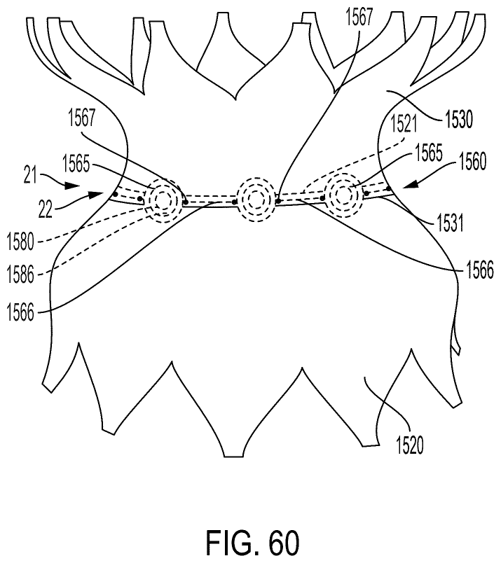

In some such embodiments, the reinforced portions 1565 of the suture/band 1560 provide secure attachment points for one or more docking station elements (shown in phantom at E), such as, for example, radiopaque markers, as described herein. According to an exemplary aspect of the present disclosure, a cover having radiopaque markers around a waist portion (e.g., any of the embodiments shown in FIGS. 46A-58D and described above), may include a band or suture extending around the waist portion, with the radiopaque markers being secured to reinforced portions of the band or suture pattern, and with unreinforced or weakened frangible portions disposed between the reinforced portions to provide for selective expansion of the waist portion by breaking the frangible portions. FIG. 60 schematically illustrates an exemplary impermeable frame covering 21 including proximal and distal portions 1520, 1530 having inner axial end portions 1521, 1531 that overlap to define a medial pocket region 22 retaining one or more radiopaque markers 1580, with a waist restricting band or suture 1560 extending circumferentially around the medial pocket region. The band/suture 1560 includes reinforced (e.g., interlocking suture) portions 1565 extending around pockets 1586 formed in the pocket region 22 to securely retain the radiopaque markers 1580, and weakened or frangible (e.g., non-interlocking suture) portions 1566 extending between the reinforced portions 1565. The pockets 1586 may be formed using a variety of suture and material configurations, including for example, any of the pocket material and suture configurations shown in FIGS. 46A-58E and described above. Knots or other such reinforcing junctions (e.g., clips, fasteners) 1567 may be provided between the reinforced portions 1565 and the frangible portions 1566 to further secure retention of the radiopaque markers 1580 in the pockets 1586.

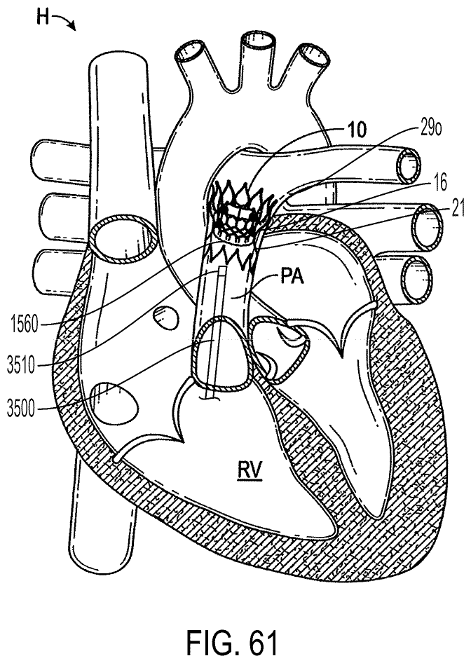

FIGS. 61-63 illustrate an exemplary method of installing an expandable prosthetic device 29 (e.g., a prosthetic valve) in a docking station 10 (e.g., any of the exemplary docking stations shown or described herein) installed (e.g., docked or deployed) in a vessel (e.g., pulmonary artery PA) of a circulatory system. In an exemplary embodiment, a first or original prosthetic device 29o is already disposed within a waist portion 16 of the docking station, with the method providing a replacement prosthetic device 29, for example, to replace an inadequately performing prosthetic valve 29o. In other embodiments, the prosthetic device may be an original (non-replacement) prosthetic device, for example, having an expanded diameter larger than a diameter of the waist portion 16. The docking station includes a cover 21 (e.g., any of the covers shown and described herein) having a circumferential band/suture 1560 that restricts the waist portion 16 of the docking station, with the band/suture having weakened or frangible portions, for example, as described above.

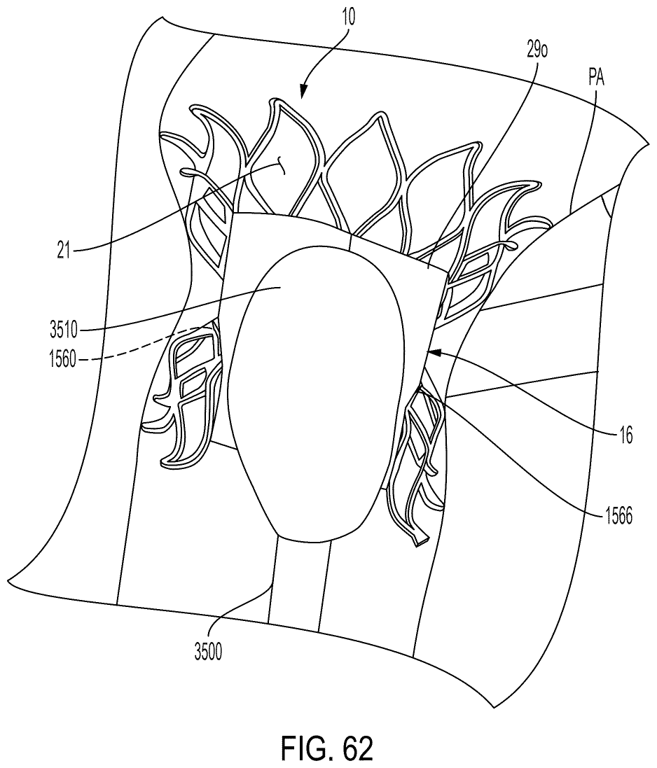

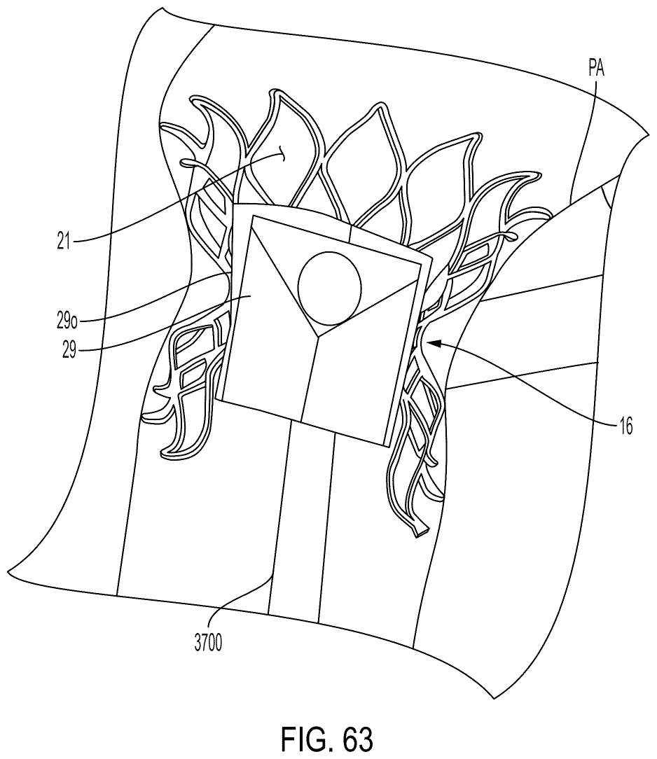

As shown in FIG. 61, an expanding tool 3510 (e.g., an inflatable noncompliant balloon) is inserted (e.g., via a first catheter 3500) into the waist portion 16 of the docking station 10, and into the original prosthetic device 29o (if included). As shown in FIG. 62, an expandable, relatively inelastic element (e.g., noncompliant balloon) of the expandable tool 3510 is expanded against the docking station waist portion 16 to break the frangible portions 1566 of the band/suture 1560, thereby expanding the waist portion of the docking station 10 and, if included, a central passage of the original prosthetic device 29o. The expandable tool 3510 is then withdrawn from the vessel for removal. As shown in FIG. 63, the prosthetic valve is inserted (e.g., via a second catheter 3700) into the expanded waist portion 16 (and into the expanded original prosthetic device 29o, if included) and is then expanded into seating engagement with the docking station waist portion and/or expanded original prosthetic device 29o.

The foregoing primarily describes embodiments of docking stations that are self-expanding. But the docking stations and/or delivery devices shown and described herein can be modified for delivery of balloon-expandable and/or mechanically-expandable docking devices, within the scope of the present disclosure. That is to say, delivering balloon-expandable and/or mechanically-expandable docking stations to an implantation location can be performed percutaneously using modified versions of the delivery devices of the present disclosure. In general terms, this includes providing a transcatheter assembly that can include a delivery sheath and/or additional sheaths as described above. In the case of balloon-expandable docking stations, the devices generally further include a delivery catheter, a balloon catheter, and/or a guide wire. A delivery catheter used in a balloon-expandable type of delivery device can define a lumen within which the balloon catheter is received. The balloon catheter, in turn, defines a lumen within which the guide wire is slidably disposed. Further, the balloon catheter includes a balloon that is fluidly connected to an inflation source. With the docking station mounted on the balloon, the transcatheter assembly is delivered through a percutaneous opening in the subject via the delivery device. Once the docking station is properly positioned, the balloon catheter is operated to inflate the balloon, thus transitioning the docking station to an expanded arrangement.

### EXAMPLES

In view of the above described implementations of the disclosed subject matter, this disclosure provides additional examples enumerated below. It should be noted that one feature of an example in isolation or more than one feature of the example taken in combination and, optionally, in combination with one or more features of one or more further examples are further examples also falling within the disclosure of this application.
Example 1. A docking station for a medical device, the docking station comprising an expandable frame extending axially from a proximal end to a distal end and having a central waist portion defining a seat; and a cover secured to the expandable frame and covering at least the waist portion of the expandable frame. The cover includes a proximal cover portion having an inner axial end disposed at the central waist portion of the expandable frame, and an outer axial end extending toward the proximal end of the expandable frame; a distal cover portion having an inner axial end disposed at the central waist portion of the expandable frame, and an outer axial end extending toward the distal end of the expandable frame, wherein the inner axial end of the distal cover portion overlaps with the inner axial end of the proximal cover portion to define a medial pocket region; a suture stitched through the medial pocket region, the suture including circumferential portions and axially extending peripheral portions defining a plurality of pockets in the medial pocket region; and a plurality of radiopaque markers, each disposed in a corresponding one of the plurality of pockets.
Example 2. The docking station of Example 1, wherein one of the proximal cover portion and the distal cover portion includes a plurality of axially inward extending flaps secured to the other of the proximal cover portion and the distal cover portion to define the plurality of pockets.
Example 3. The docking station of Example 1, wherein the proximal and distal cover portions each include a plurality of axially inward extending flaps that align to define the plurality of pockets.
Example 4. The docking station of any of Examples 1-3, wherein each pocket includes a crossing suture portion extending through an aperture in the corresponding radiopaque marker to secure the radiopaque marker within the pocket.
Example 5. The docking station of Example 4, wherein the crossing suture portion includes interlocking stitches.
Example 6. The docking station of any of Examples 4 and 5, wherein the crossing suture portion is continuous with the peripheral suture portion.
Example 7. The docking station of any of Examples 4 and 5, wherein the suture is a first suture and the crossing suture portion is defined by a second suture separate from the first suture.
Example 8. The docking station of any of Examples 4-7, wherein the crossing suture portion further extends through a second aperture in the corresponding radiopaque marker to secure the radiopaque marker within the pocket.
Example 9. The docking station of any of Examples 1-8, wherein the suture includes a first suture pattern and a second suture pattern.
Example 10. The docking station of Example 9, wherein the first and second suture patterns include non-interlocking stitches in the peripheral portions of the suture.
Example 11. The docking station of Example 9, wherein the first and second suture patterns include interlocking stitches in the peripheral portions of the suture.
Example 12. The docking station of any of Examples 9-11, wherein the first and second suture patterns include interlocking stitches in the circumferential portions of the suture.
Example 13. The docking station of any of Examples 9-11, wherein the first and second suture patterns include non-interlocking stitches in the circumferential portions of the suture.
Example 14. The docking station of any of Examples 9-13, wherein the first and second suture patterns include interlocking crossing stitches extending through an aperture in the radiopaque marker.
Example 15. The docking station of any of Examples 1-14, wherein the circumferential portions of the suture are axially aligned with the pockets.
Example 16. The docking station of any of Examples 1-14, wherein the circumferential portions of the suture are axially offset from the pockets.
Example 17. The docking station of any of Examples 1-16, wherein the suture restricts the central waist portion to a first diameter.
Example 18. The docking station of Example 17, wherein the suture includes a plurality of frangible portions disposed between a plurality of reinforced portions, such that when an expansion tool is inserted into the waist portion of the expandable frame and expanded to a size larger than the first diameter, the frangible portions of the suture break without breakage of the reinforced portions to expand the waist portion to a second diameter larger than the first diameter.
Example 19. The docking station of Example 18, wherein the plurality of frangible portions is disposed in the circumferential portions of the suture.
Example 20. The docking station of any of Examples 18 and 19, wherein the plurality of reinforced portions includes the peripheral portions of the suture.
Example 21. The docking station of any of Examples 18-20, the cover further comprises reinforcing junctions between the plurality of frangible portions and the plurality of reinforced portions.
Example 22. the Docking Station of Example 21, Wherein the Reinforcing junctions comprise suture knots.
Example 23. A docking station for a medical device includes an expandable frame extending axially from a proximal end to a distal end and having a central waist portion defining a seat; and a cover secured to the expandable frame and covering at least the waist portion of the expandable frame. The cover includes a proximal cover portion having an inner axial end disposed at the central waist portion of the expandable frame, and an outer axial end extending toward the proximal end of the expandable frame; a distal cover portion having an inner axial end disposed at the central waist portion of the expandable frame, and an outer axial end extending toward the distal end of the expandable frame, wherein the inner axial ends of the proximal and distal cover portions include axially inward extending flaps that overlap to define a plurality of pockets between the proximal cover portion and the distal cover portion; and a plurality of radiopaque markers, each disposed in a corresponding one of the plurality of pockets.
Example 24. The docking station of Example 23, wherein the axial inner ends of the proximal and distal covers overlap, the cover further including a suture stitched through the overlapping axial inner ends.
Example 25. The docking station of Example 24, wherein the suture includes circumferential portions and axially extending peripheral portions extending through the plurality of pockets.
Example 26. The docking station of Example 25, wherein each pocket includes a crossing suture portion extending through an aperture in the corresponding radiopaque marker to secure the radiopaque marker within the pocket.
Example 27. The docking station of Example 26, wherein the crossing suture portion includes interlocking stitches.
Example 28. The docking station of any of Examples 26 and 27, wherein the crossing suture portion is continuous with the peripheral suture portion.
Example 29. The docking station of any of Examples 26 and 27, wherein the suture is a first suture and the crossing suture portion is defined by a second suture separate from the first suture.
Example 30. The docking station of any of Examples 26-29, wherein the crossing suture portion further extends through a second aperture in the corresponding radiopaque marker to secure the radiopaque marker within the pocket.
Example 31. The docking station of any of Examples 25-30, wherein the suture includes a first suture pattern and a second suture pattern.
Example 32. The docking station of Example 31, wherein the first and second suture patterns include non-interlocking stitches in the peripheral portions of the suture.
Example 33. The docking station of Example 31, wherein the first and second suture patterns include interlocking stitches in the peripheral portions of the suture.
Example 34. The docking station of any of Examples 31-33, wherein the first and second suture patterns include interlocking stitches in the circumferential portions of the suture.
Example 35. The docking station of any of Examples 31-33, wherein the first and second suture patterns include non-interlocking stitches in the circumferential portions of the suture.
Example 36. The docking station of any of Examples 31-35, wherein the first and second suture patterns include interlocking crossing stitches extending through an aperture in the radiopaque marker.
Example 37. The docking station of any of Examples 25-36, wherein the circumferential portions of the suture are axially aligned with the pockets.
Example 38. The docking station of any of Examples 25-36, wherein the circumferential portions of the suture are axially offset from the pockets.
Example 39. The docking station of any of Examples 25-38, wherein the suture restricts the central waist portion to a first diameter.
Example 40. The docking station of Example 39, wherein the suture includes a plurality of frangible portions disposed between a plurality of reinforced portions, such that when an expansion tool is inserted into the waist portion of the expandable frame and expanded to a size larger than the first diameter, the frangible portions of the suture break without breakage of the reinforced portions to expand the waist portion to a second diameter larger than the first diameter.
Example 41. The docking station of Example 40, wherein the plurality of frangible portions is disposed in the circumferential portions of the suture.
Example 42. The docking station of any of Examples 40 and 41, wherein the plurality of reinforced portions includes the peripheral portions of the suture.
Example 43. The docking station of any of Examples 40-42, further comprising reinforcing junctions between the plurality of frangible portions and the plurality of reinforced portions.
Example 44. The docking station of Example 43, wherein the reinforcing junctions comprise suture knots.
Example 45. A cover for a docking station frame, wherein the cover comprises the cover of any of Examples 1-44.
Example 46. A method of forming a cover for a docking station frame, the method comprising overlapping an inner axial end of a proximal cover portion with an inner axial end of a distal cover portion to define a medial pocket region; positioning a plurality of radiopaque markers in the medial pocket region between the proximal cover portion and the distal cover portion; and sewing a suture through the medial pocket region, the suture including circumferential portions and axially extending peripheral portions defining a plurality of pockets between the inner axial ends of the proximal and distal cover portions, with each of the plurality of radiopaque markers being disposed in a corresponding one of the plurality of pockets.
Example 47. The method of Example 46, wherein the cover comprises the cover of any of Examples 1-44.
Example 48. A docking station for a medical device, the docking station comprising an expandable frame including a waist portion defining a seat for retaining an expandable medical device; and a cover secured to the expandable frame and covering at least the waist portion of the expandable frame, the cover including a circumferential band extending around the waist portion of the expandable frame to restrict expansion of the waist portion to a first diameter. The circumferential band includes a plurality of frangible portions disposed between a plurality of reinforced portions, such that when an expansion tool is inserted into the waist portion of the expandable frame and expanded to a size larger than the first diameter, the frangible portions of the circumferential band break without breakage of the reinforced portions to expand the waist portion to a second diameter larger than the first diameter.
Example 49. The docking station of Example 48, wherein the circumferential band comprises a suture.
Example 50. The docking station of Example 49, wherein the cover comprises the cover of any of Examples 1-44.
Example 51. A method of installing an expandable transcatheter valve in a docking station having an expandable frame including a waist portion defining a seat for retaining an expandable medical device, and a cover secured to the expandable frame and covering at least the waist portion of the expandable frame, the cover including a circumferential band extending around the waist portion of the expandable frame to restrict expansion of the waist portion to a first diameter. The method comprises inserting an expansion tool into the waist portion of the expandable frame; expanding the expansion tool to break frangible portions of the circumferential band, thereby expanding the waist portion from the first diameter to a second diameter larger than the first diameter; and expanding the expandable transcatheter valve within the expanded waist portion of the docking station frame to secure the expandable transcatheter valve within the docking station.
Example 52. The method of Example 51, wherein the docking station further includes an original transcatheter valve installed in the waist portion of the docking station frame, wherein inserting the expansion tool into the waist portion of the expandable frame comprises inserting the expansion tool into a central passage of the original transcatheter valve; expanding the expansion tool comprises expanding the central passage of the original transcatheter valve; and expanding the expandable transcatheter valve within the expanded waist portion of the docking station frame comprises expanding the expandable transcatheter valve into seating engagement with the central passage of the original transcatheter valve.
Example 53. The method of any of Examples 51 and 52, wherein the circumferential band comprises a suture.
Example 54. The method of Example 53, wherein the cover comprises the cover of any of Examples 1-44.

In view of the many possible embodiments to which the principles of the disclosed invention may be applied, it should be recognized that the illustrated embodiments are only preferred examples of the invention and should not be taken as limiting the scope of the invention. All combinations or sub-combinations of features of the foregoing exemplary embodiments are contemplated by this application. The scope of the invention is defined by the following claims. We therefore claim as our invention all that comes within the scope and spirit of these claims.

While various inventive aspects, concepts and features of the inventions may be described and illustrated herein as embodied in combination in the exemplary embodiments, these various aspects, concepts and features may be used in many alternative embodiments, either individually or in various combinations and sub-combinations thereof. Unless expressly excluded herein all such combinations and sub-combinations are intended to be within the scope of the present inventions. Still further, while various alternative embodiments as to the various aspects, concepts and features of the inventions—such as alternative materials, structures, configurations, methods, circuits, devices and components, alternatives as to form, fit and function, and so on—may be described herein, such descriptions are not intended to be a complete or exhaustive list of available alternative embodiments, whether presently known or later developed. Those skilled in the art may readily adopt one or more of the inventive aspects, concepts or features into additional embodiments and uses within the scope of the present inventions even if such embodiments are not expressly disclosed herein. Additionally, even though some features, concepts or aspects of the inventions may be described herein as being a preferred arrangement or method, such description is not intended to suggest that such feature is required or necessary unless expressly so stated. Still further, exemplary or representative values and ranges may be included to assist in understanding the present disclosure, however, such values and ranges are not to be construed in a limiting sense and are intended to be critical values or ranges only if so expressly stated. Parameters identified as “approximate” or “about” a specified value are intended to include the specified value, values within 5% of the specified value, and values within 10% of the specified value, unless expressly stated otherwise. Further, it is to be understood that the drawings accompanying the present disclosure may, but need not, be to scale, and therefore may be understood as teaching various ratios and proportions evident in the drawings. Moreover, while various aspects, features and concepts may be expressly identified herein as being inventive or forming part of an invention, such identification is not intended to be exclusive, but rather there may be inventive aspects, concepts and features that are fully described herein without being expressly identified as such or as part of a specific invention, the inventions instead being set forth in the appended claims. Descriptions of exemplary methods or processes are not limited to inclusion of all steps as being required in all cases, nor is the order that the steps are presented to be construed as required or necessary unless expressly so stated.

What is claimed is:

1. A docking station for a medical device, the docking station comprising:
an expandable frame extending axially from a proximal end to a distal end and having a waist portion defining a seat;
a cover secured to the expandable frame and covering at least the waist portion of the expandable frame, the cover including:
a proximal cover portion having an inner axial end disposed at the central waist portion of the expandable frame, and an outer axial end extending toward the proximal end of the expandable frame;
a distal cover portion having an inner axial end disposed at the central waist portion of the expandable frame, and an outer axial end extending toward the distal end of the expandable frame, wherein the inner axial end of the distal cover portion overlaps with the inner axial end of the proximal cover portion to define a medial pocket region;
a suture stitched through the medial pocket region, the suture including circumferential portions and axially extending peripheral portions defining a plurality of pockets in the medial pocket region; and
a plurality of radiopaque markers, each disposed in a corresponding one of the plurality of pockets.

2. The docking station of claim 1, wherein one of the proximal cover portion and the distal cover portion includes a plurality of axially inward extending flaps secured to the other of the proximal cover portion and the distal cover portion to define the plurality of pockets.

3. The docking station of claim 1, wherein the proximal and distal cover portions each include a plurality of axially inward extending flaps that align to define the plurality of pockets.

4. The docking station of claim 1, wherein each pocket includes a crossing suture portion extending through an aperture in the corresponding radiopaque marker to secure the radiopaque marker within the pocket.

5. The docking station of claim 4, wherein the crossing suture portion further extends through a second aperture in the corresponding radiopaque marker to secure the radiopaque marker within the pocket.

6. The docking station of claim 4, wherein the suture includes a first suture pattern and a second suture pattern.

7. The docking station of claim 6, wherein the first and second suture patterns include non-interlocking stitches in the peripheral portions of the suture.

8. The docking station of claim 6, wherein the first and second suture patterns include interlocking crossing stitches extending through the aperture in the radiopaque marker.

9. The docking station of claim 1, wherein the suture restricts the central waist portion to a first diameter.

10. The docking station of claim 9, wherein the suture includes a plurality of frangible portions disposed between a plurality of reinforced portions, such that when an expansion tool is inserted into the waist portion of the expandable frame and expanded to a size larger than the first diameter, the plurality of frangible portions of the suture break without breakage of the reinforced portions to expand the waist portion to a second diameter larger than the first diameter.

11. The docking station of claim 10, wherein the cover further comprises reinforcing junctions between the plurality of frangible portions and the plurality of reinforced portions.

12. A docking station for a medical device, the docking station comprising:
an expandable frame extending axially from a proximal end to a distal end and having a waist portion defining a seat;
a cover secured to the expandable frame and covering at least the waist portion of the expandable frame, the cover including:
a proximal cover portion having an inner axial end disposed at the central waist portion of the expandable frame, and an outer axial end extending toward the proximal end of the expandable frame;
a distal cover portion having an inner axial end disposed at the central waist portion of the expandable frame, and an outer axial end extending toward the distal end of the expandable frame, wherein the inner axial ends of the proximal and distal cover portions include axially inward extending flaps that overlap to define a plurality of pockets between the proximal cover portion and the distal cover portion; and
a plurality of radiopaque markers, each disposed in a corresponding one of the plurality of pockets.

13. The docking station of claim 12, wherein the inner axial ends of the proximal and distal covers overlap, the cover further including a suture stitched through the overlapping axial inner ends.

14. The docking station of claim 13, wherein the suture includes circumferential portions and axially extending peripheral portions extending through the plurality of pockets.

15. The docking station of claim 14, wherein each pocket includes a crossing suture portion extending through an aperture in the corresponding radiopaque marker to secure the radiopaque marker within the pocket.

16. A docking station for a medical device, the docking station comprising:
an expandable frame including a waist portion defining a seat for retaining an expandable medical device;
a cover secured to the expandable frame and covering at least the waist portion of the expandable frame, the cover including a circumferential band extending around the waist portion of the expandable frame to restrict expansion of the waist portion to a first diameter; and
wherein the circumferential band includes a plurality of frangible portions disposed between a plurality of reinforced portions, such that when an expansion tool is inserted into the waist portion of the expandable frame and expanded to a size larger than the first diameter, the plurality of frangible portions of the circumferential band break without breakage of the reinforced portions to expand the waist portion to a second diameter larger than the first diameter.

17. The docking station of claim 16, wherein the circumferential band comprises a suture.

18. The docking station of claim 17, wherein the suture includes a first suture pattern and a second suture pattern.

19. The docking station of claim 18, wherein the first and second suture patterns include non-interlocking stitches in the peripheral portions of the suture.

20. The docking station of claim 18, further comprising a radiopaque marker having an aperture, and wherein the first and second suture patterns include interlocking crossing stitches extending through the aperture in the radiopaque marker.
